(19) United States
(12) Patent Application Publication
(10) Pub. No.: US 2025/0082681 A1
(43) Pub. Date: Mar. 13, 2025

(54) ANTIBODIES AGAINST CLL1 AND CONSTRUCTS THEREOF

(71) Applicant: Nanjing Legend Biotech Co., Ltd., Nanjing (CN)

(72) Inventors: Yafeng ZHANG, Nanjing (CN); Yanliang ZHU, Nanjing (CN); Nannan ZHOU, Nanjing (CN); Shuai YANG, Nanjing (CN); Shu WU, Nanjing (CN)

(21) Appl. No.: 18/580,123

(22) PCT Filed: Aug. 1, 2022
(86) PCT No.: PCT/CN2022/109528

§ 371 Date: Jan. 17, 2024

(30) Foreign Application Priority Data
Jul. 30, 2021 (WO) PCT/CN2021/109838

(51) Int. Cl. A61K 35/17; A61K 39/00; A61K 47/68; A61P 35/00; C07K 16/28; C12N 5/0783; C12N 15/86

(52) U.S. Cl. CPC A61K 35/17; A61K 40/11; A61K 40/31; A61K 40/421; A61K 40/4224; A61K 47/6849; A61P 35/00; C07K 16/2803; C07K 16/2851; C12N 5/0636; C12N 15/86; A61K 2239/13; A61K 2239/17; A61K 2239/21; A61K 2239/22; A61K 2239/48; C07K 2317/24; C07K 2317/565; C07K 2317/622; C12N 2740/15043

(57) ABSTRACT

Provided herein are antibodies and antigen-binding fragment thereof targeting CLL1, and chimeric antigen receptors (e.g., monovalent CAR, and multivalent CAR including bi-epitope CAR) having one or more anti-CLL1 antigen-binding fragments thereof. Further provided are engineered immune effector cells (e.g., T cells) expressing the chimeric antigen receptors and methods of use thereof.

### CROSS REFERENCE TO RELATED APPLICATION

This application claims priority benefits of International Application No. PCT/CN2021/109838 filed Jul. 30, 2021, entitled “ANTIBODIES AGAINST CLL1 AND CONSTRUCTS THEREOF”, the contents of which is incorporated herein by reference in its entirety.

### SEQUENCE STATEMENT

The content of the following submission on ASCII text file is incorporated herein by reference in its entirety: a computer readable form (CRF) of the Sequence Listing (file name: P11219-PCT.220801.Sequence listing.xml, date recorded: Aug. 1, 2022, size: 199 kilobytes).

### TECHNICAL FIELD

This disclosure relates to antibodies targeting CLL1, chimeric antigen receptors targeting CLL1, and methods of use thereof.

### BACKGROUND

Acute myeloid leukemia (AML) is a cancer of the myeloid line of blood cells, characterized by the rapid growth of immature blood cells (“blasts”) that build up in the bone marrow and blood and interfere with normal blood cells. AML may spread to other organs, such as the liver, spleen, and brain. Clinical symptoms of AML include fatigue, shortness of breath, easy bruising and bleeding, and increased risk of infection. Without treatment, AML progresses rapidly and is typically fatal within weeks or months (De Kouchkovsky, I. et al., 2016, Blood Cancer J 6(7): e441). AML has several subtypes for which treatments and outcomes may vary. Typically, AML is initially treated with chemotherapy, sometimes along with a targeted therapy drug. Patients may then go on to receive a stem cell transplant, additional chemotherapy, surgery, or radiation therapy. AML most commonly occurs in older adults, some of whom are not healthy enough to receive intensive chemotherapy and thus have poor clinical outcome (Dohner, H., et al., 2015, N Engl J Med 373(12): 1136-1152; and Medinger, M., et al., 2019, Ther Umsch 76(9): 481-486). Almost all patients under current therapies of AML eventually relapse. Thus, there exists a need for an effective immunotherapeutic agent to treat AML.

### SUMMARY

The disclosure relates to antibodies and antigen-binding fragments thereof that bind to CLL1. The disclosure also relates to anti-CLL1 CAR-T cell therapy for the treatment of cancer patients with CLL1-positive cancer, including e.g., acute myeloid leukemia (AML). Genetically engineered T cells can recognize and attack target cells. These T cells can be isolated from the host and genetically modified using e.g., suitable virus mediated or non-viral means of transfection. Thereafter, the modified T cells can be infused back into the patients as adoptive cell therapy.

In one aspect, the disclosure relates to an antibody or antigen-binding fragment thereof that binds to CLL1 (C-type lectin-like molecule 1, or C-type lectin domain family 12 member A), comprising: a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence, wherein the selected VH CDRs 1, 2, and 3 amino acid sequences and the selected VL CDRs 1, 2, and 3 amino acid sequences are one of the following: (1) the selected VH CDRs 1, 2, 3 amino acid sequences are set forth in SEQ ID NOs: 1, 2, and 3 respectively, and the selected VL CDRs 1, 2, 3 amino acid sequences are set forth in SEQ ID NOs: 4, 5, and 6 respectively; (2) the selected VH CDRs 1, 2, 3 amino acid sequences are set forth in SEQ ID NOs: 11, 12, and 13 respectively, and the selected VL CDRs 1, 2, 3 amino acid sequences are set forth in SEQ ID NOs: 14, 15, and 16 respectively; (3) the selected VH CDRs 1, 2, 3 amino acid sequences are set forth in SEQ ID NOs: 21, 22, and 23 respectively, and the selected VL CDRs 1, 2, 3 amino acid sequences are set forth in SEQ ID NOs: 24, 25, and 26 respectively; (4) the selected VH CDRs 1, 2, 3 amino acid sequences are set forth in SEQ ID NOs: 31, 32, and 33 respectively, and the selected VL CDRs 1, 2, 3 amino acid sequences are set forth in SEQ ID NOs: 34, 35, and 36 respectively; (5) the selected VH CDRs 1, 2, 3 amino acid sequences are set forth in SEQ ID NOs: 41, 42, and 43 respectively, and the selected VL CDRs 1, 2, 3 amino acid sequences are set forth in SEQ ID NOs: 44, 45, and 46 respectively; (6) the selected VH CDRs 1, 2, 3 amino acid sequences are set forth in SEQ ID NOs: 51, 52, and 53 respectively, and the selected VL CDRs 1, 2, 3 amino acid sequences are set forth in SEQ ID NOs: 54, 55, and 56 respectively; (7) the selected VH CDRs 1, 2, 3 amino acid sequences are set forth in SEQ ID NOs: 61, 62, and 63 respectively, and the selected VL CDRs 1, 2, 3 amino acid sequences are set forth in SEQ ID NOs: 64, 65, and 66 respectively; (8) the selected VH CDRs 1, 2, 3 amino acid sequences are set forth in SEQ ID NOs: 71, 72, and 73 respectively, and the selected VL CDRs 1, 2, 3 amino acid sequences are set forth in SEQ ID NOs: 74, 75, and 76 respectively; (9) the selected VH CDRs 1, 2, 3 amino acid sequences are set forth in SEQ ID NOs: 81, 82, and 83 respectively, and the selected VL CDRs 1, 2, 3 amino acid sequences are set forth in SEQ ID NOs: 84, 85, and 86 respectively; (10) the selected VH CDRs 1, 2, 3 amino acid sequences are set forth in SEQ ID NOs: 91, 92, and 93 respectively, and the selected VL CDRs 1, 2, 3 amino acid sequences are set forth in SEQ ID NOs: 94, 95, and 96 respectively; (11) the selected VH CDRs 1, 2, 3 amino acid sequences are set forth in SEQ ID NOs: 101, 102, and 103 respectively, and the selected VL CDRs 1, 2, 3 amino acid sequences are set forth in SEQ ID NOs: 104, 105, and 106 respectively; (12) the selected VH CDRs 1, 2, 3 amino acid sequences are set forth in SEQ ID NOs: 111, 112, and 113 respectively, and the selected VL CDRs 1, 2, 3 amino acid sequences are set forth in SEQ ID NOs: 114, 115, and 116 respectively; and (13) the selected VH CDRs 1, 2, 3 amino acid sequences are set forth in SEQ ID NOs: 121, 122, and 123 respectively, and the selected VL CDRs 1, 2, 3 amino acid sequences are set forth in SEQ ID NOs: 124, 125, and 126 respectively.

In one aspect, the disclosure relates to an antibody or antigen-binding fragment thereof that binds to CLL1, comprising a heavy chain variable region (VH) comprising an amino acid sequence that is at least 80%, 85%, 90%, 95% or 100% identical to a selected VH sequence, and a light chain variable region (VL) comprising an amino acid sequence that is at least 80%, 85%, 90%, 95% or 100% identical to a selected VL sequence, wherein the selected VH sequence and the selected VL sequence are one of the following: (1) the selected VH sequence is SEQ ID NO: 10 and the selected VL sequence is SEQ ID NO: 9; (2) the selected VH sequence is SEQ ID NO: 20, and the selected VL sequence is SEQ ID NO: 19; (3) the selected VH sequence is SEQ ID NO: 30, and the selected VL sequence is SEQ ID NO: 29; (4) the selected VH sequence is SEQ ID NO: 40, and the selected VL sequence is SEQ ID NO: 39; (5) the selected VH sequence is SEQ ID NO: 50, and the selected VL sequence is SEQ ID NO: 49; (6) the selected VH sequence is SEQ ID NO: 60, and the selected VL sequence is SEQ ID NO: 59; (7) the selected VH sequence is SEQ ID NO: 70, and the selected VL sequence is SEQ ID NO: 69; (8) the selected VH sequence is SEQ ID NO: 80, and the selected VL sequence is SEQ ID NO: 79; (9) the selected VH sequence is SEQ ID NO: 90, and the selected VL sequence is SEQ ID NO: 89; (10) the selected VH sequence is SEQ ID NO: 100, and the selected VL sequence is SEQ ID NO: 99; (11) the selected VH sequence is SEQ ID NO: 110, and the selected VL sequence is SEQ ID NO: 109; (12) the selected VH sequence is SEQ ID NO: 120, and the selected VL sequence is SEQ ID NO: 119; and (13) the selected VH sequence is SEQ ID NO: 130, and the selected VL sequence is SEQ ID NO: 129.

In one aspect, the disclosure relates to an antibody or antigen-binding fragment thereof that binds to CLL1, comprising a heavy chain variable region (VH) comprising VH CDR1, VH CDR2, and VH CDR3 that are identical to VH CDR1, VH CDR2, and VH CDR3 of a selected VH sequence, and a light chain variable region (VL) comprising VL CDR1, VL CDR2, and VL CDR3 that are identical to VL CDR1, VL CDR2, and VL CDR3 of a selected VL sequence, wherein the selected VH sequence and the selected VL sequence are one of the following: (1) the selected VH sequence is SEQ ID NO: 10 and the selected VL sequence is SEQ ID NO: 9; (2) the selected VH sequence is SEQ ID NO: 20, and the selected VL sequence is SEQ ID NO: 19; (3) the selected VH sequence is SEQ ID NO: 30, and the selected VL sequence is SEQ ID NO: 29; (4) the selected VH sequence is SEQ ID NO: 40, and the selected VL sequence is SEQ ID NO: 39; (5) the selected VH sequence is SEQ ID NO: 50, and the selected VL sequence is SEQ ID NO: 49; (6) the selected VH sequence is SEQ ID NO: 60, and the selected VL sequence is SEQ ID NO: 59; (7) the selected VH sequence is SEQ ID NO: 70, and the selected VL sequence is SEQ ID NO: 69; (8) the selected VH sequence is SEQ ID NO: 80, and the selected VL sequence is SEQ ID NO: 79; (9) the selected VH sequence is SEQ ID NO: 90, and the selected VL sequence is SEQ ID NO: 89; (10) the selected VH sequence is SEQ ID NO: 100, and the selected VL sequence is SEQ ID NO: 99; (11) the selected VH sequence is SEQ ID NO: 110, and the selected VL sequence is SEQ ID NO: 109; (12) the selected VH sequence is SEQ ID NO: 120, and the selected VL sequence is SEQ ID NO: 119; and (13) the selected VH sequence is SEQ ID NO: 130, and the selected VL sequence is SEQ ID NO: 129.

In some embodiments, the antibody or antigen-binding fragment is a single-chain variable fragment (scFv).

In some embodiments, the antibody or antigen-binding fragment specifically binds to a human CLL1 peptide comprising a sequence that is at least 80%, 85%, 90%, 95%, or 100% identical to the amino acid sequence of SEQ ID NO: 147.

In some embodiments, the antibody or antigen-binding fragment specifically binds to the extracellular domain (ECD) of human CLL1.

In some embodiments, the antibody or antigen-binding fragment is a humanized antibody or antigen-binding fragment thereof.

In some embodiments, the antibody or antigen-binding fragment is a chimeric antibody or antigen-binding fragment thereof or a human antibody or antigen-binding fragment thereof.

In one aspect, the disclosure relates to an antibody or antigen-binding fragment thereof that cross-competes with any one of the antibody or antigen-binding fragment thereof described herein.

In one aspect, the disclosure relates to an antibody-drug conjugate comprising the antibody or antigen-binding fragment thereof described herein.

In one aspect, the disclosure relates to a pharmaceutical composition comprising the antibody or antigen-binding fragment thereof described herein, or the antibody-drug conjugate described herein, and a pharmaceutically acceptable carrier.

In one aspect, the disclosure relates to a nucleic acid comprising a polynucleotide encoding the antibody or antigen-binding fragment thereof described herein.

In one aspect, the disclosure relates to a vector comprising the nucleic acid described herein.

In one aspect, the disclosure relates to a cell comprising the vector described herein.

In one aspect, the disclosure relates to a method of producing an antibody or an antigen-binding fragment thereof, the method comprising: (a) culturing the cell comprising the vector described herein under conditions sufficient for the cell to produce the antibody or the antigen-binding fragment thereof, and (b) collecting the antibody or the antigen-binding fragment thereof produced by the cell.

In one aspect, the disclosure relates to an engineered receptor comprising the antigen-binding fragment thereof described herein.

In some embodiments, the engineered receptor further comprises a transmembrane region, and an intracellular signaling domain.

In some embodiments, the engineered receptor is a chimeric antigen receptor (“CAR”).

In some embodiments, the engineered receptor further comprises a hinge region.

In some embodiments, the transmembrane region comprises a transmembrane region of CD4, CD8, and/or CD28, or a portion thereof.

In some embodiments, the intracellular signaling domain comprises a primary intracellular signaling sequence of an immune effector cell.

In some embodiments, the intracellular signaling domain is or comprises a functional signaling domain of CD3 zeta.

In some embodiments, the intracellular signaling domain further comprises a costimulatory signaling domain.

In some embodiments, the costimulatory signaling domain comprises a functional signaling domain from a protein selected from the group consisting of a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1, CD11a/CD18, 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, CD19a, and a CD83 ligand.

In some embodiments, the costimulatory signaling domain comprises an intracellular signaling domain of 4-1BB and/or CD28.

In some embodiments, the engineered receptor comprises a signal peptide.

In some embodiments, the signal peptide is at least 80%, 85%, 90%, 95% or 100% identical to SEQ ID NO: 146.

In some embodiments, the engineered receptor comprises an amino acid sequence set forth in any one of SEQ ID NOs: 7, 17, 27, 37, 47, 57, 67, 77, 87, 97, 107, 117, and 127, or an amino acid sequence that is at least 90%, 91%, 92%, 93%, 94%, 95%, 96%, 97%, 98%, 99% or 100% identical to any one of SEQ ID NOs: 7, 17, 27, 37, 47, 57, 67, 77, 87, 97, 107, 117, and 127.

In some embodiments, the amino acid sequence is identical to any one of SEQ ID NOs: 7, 17, 27, 37, 47, 57, 67, 77, 87, 97, 107, 117, and 127.

In one aspect, the disclosure relates to an engineered receptor comprising: (a) a first antigen-binding fragment thereof of any one of the antigen-binding fragments described herein; and (b) a second antigen-binding fragment thereof that binds to CD33.

In some embodiments, the first antigen-binding fragment thereof and the second antigen-binding fragment thereof are connected via a linker.

In some embodiments, the engineered receptor further comprises a transmembrane region, and an intracellular signaling domain.

In some embodiments, the engineered receptor is a chimeric antigen receptor (“CAR”).

In some embodiments, the engineered receptor further comprises a hinge region.

In some embodiments, the transmembrane region comprises a transmembrane region of CD4, CD8, and/or CD28, or a portion thereof.

In some embodiments, the intracellular signaling domain comprises a primary intracellular signaling sequence of an immune effector cell.

In some embodiments, the intracellular signaling domain is or comprises a functional signaling domain of CD3 zeta.

In some embodiments, the intracellular signaling domain further comprises a costimulatory signaling domain.

In some embodiments, the costimulatory signaling domain comprises a functional signaling domain from a protein selected from the group consisting of a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1, CD11a/CD18, 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, CD19a, and a CD83 ligand.

In some embodiments, the costimulatory signaling domain comprises an intracellular signaling domain of 4-1BB and/or CD28.

In some embodiments, the engineered receptor comprises a signal peptide. In some embodiments, the signal peptide is at least 80%, 85%, 90%, 95% or 100% identical to SEQ ID NO: 146 In some embodiments, the engineered receptor is a chimeric T cell receptor (“cTCR”).

In some embodiments, the transmembrane domain is derived from the transmembrane domain of a TCR subunit selected from the group consisting of TCRα, TCRβ, TCRγ, TCRδ, CD3γ, CD3ε, and CD3δ.

In some embodiments, the transmembrane domain is derived from the transmembrane domain of CD3ε.

In some embodiments, the intracellular signaling domain is derived from the intracellular signaling domain of a TCR subunit selected from the group consisting of TCRα, TCRβ, TCRγ, TCRδ, CD3γ, CD3ε, and CD3δ.

In some embodiments, the intracellular signaling domain is derived from the intracellular signaling domain of CD3ε.

In some embodiments, the engineered receptor described herein further comprising at least a portion of an extracellular domain of a TCR subunit.

In some embodiments, the antigen binding fragment is fused to the N-terminus of CD3ε (“eTCR”).

In one aspect, the disclosure relates to a dual receptor system comprising: (a) a first engineered receptor comprising a first antigen-binding fragment thereof that can be any one of the antigen-binding fragments described herein (e.g., an antigen-binding fragment that binds to CLL1); and (b) a second engineered receptor comprising a second antigen-binding fragment thereof that binds to CD33.

In some embodiments, each of the first engineered receptor and the second engineered receptor further comprises a transmembrane region, and an intracellular signaling domain.

In some embodiments, the first engineered receptor and the second engineered receptor are both chimeric antigen receptors (“CARs”).

In some embodiments, each of the first engineered receptor and the second engineered receptor further comprises a hinge region.

In some embodiments, the transmembrane region comprises a transmembrane region of CD4, CD8, and/or CD28, or a portion thereof.

In some embodiments, the intracellular signaling domain comprises a primary intracellular signaling sequence of an immune effector cell.

In some embodiments, the intracellular signaling domain is or comprises a functional signaling domain of CD3 zeta.

In some embodiments, the intracellular signaling domain further comprises a costimulatory signaling domain.

In some embodiments, the costimulatory signaling domain comprises a functional signaling domain from a protein selected from the group consisting of a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1, CD11a/CD18, 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, CD19a, and a CD83 ligand.

In some embodiments, the costimulatory signaling domain comprises an intracellular signaling domain of 4-1BB and/or CD28.

In some embodiments, each of the first engineered receptor and the second engineered receptor comprises a signal peptide.

In some embodiments, the signal peptide is at least 80%, 85%, 90%, 95% or 100% identical to SEQ ID NO: 146.

In some embodiments, the first engineered receptor comprise an amino acid sequence set forth in any one of SEQ ID NOs: 7, 17, 27, 37, 47, 57, 67, 77, 87, 97, 107, 117, and 127, or an amino acid sequence that is at least 90%, 91%, 92%, 93%, 94%, 95%, 96%, 97%, 98%, 99% or 100% identical to 7, 17, 27, 37, 47, 57, 67, 77, 87, 97, 107, 117, and 127.

In some embodiments, the second engineered receptor comprises an amino acid sequence set forth in SEQ ID NO: 159, 163, 167 or 171, or an amino acid sequence that is at least 90%, 91%, 92%, 93%, 94%, 95%, 96%, 97%, 98%, 99% or 100% identical to SEQ ID NO: 159, 163, 167 or 171.

In some embodiments, the first engineered receptor comprises an amino acid sequence set forth in SEQ ID NO: 27 or 97, or an amino acid sequence that is at least 90%, 91%, 92%, 93%, 94%, 95%, 96%, 97%, 98%, 99% or 100% identical to SEQ ID NO: 27 or 97.

In some embodiments, the second engineered receptor comprises an amino acid sequence set forth in SEQ ID NO: 163, and the first engineered receptor comprises an amino acid sequence set forth in SEQ ID NO: 97.

In some embodiments, the second engineered receptor comprises an amino acid sequence set forth in SEQ ID NO: 163, and the first engineered receptor comprises an amino acid sequence set forth in SEQ ID NO: 27.

In some embodiments, the second engineered receptor comprises an amino acid sequence set forth in SEQ ID NO: 171, and the first engineered receptor comprises an amino acid sequence set forth in SEQ ID NO: 97.

In some embodiments, the second engineered receptor comprises an amino acid sequence set forth in SEQ ID NO: 171, and the first engineered receptor comprises an amino acid sequence set forth in SEQ ID NO: 27.

In some embodiments, the first engineered receptor and second engineered receptor are chimeric T cell receptors (“cTCRs”).

In one aspect, the disclosure relates to a polynucleotide encoding the engineered receptor of any one of the engineered receptors or dual receptor systems described herein.

In some embodiments, the polynucleotide described herein encodes a polypeptide comprising an amino acid sequence of any one of SEQ ID NOs: 132-145, or an amino acid sequence that is at least 90%, 91%, 92%, 93%, 94%, 95%, 96%, 97%, 98%, 99% or 100% identical to SEQ ID NOs: 132-145.

In one aspect, the disclosure relates to a vector comprising the polynucleotide described herein.

In some embodiments, the vector is a viral vector.

In one aspect, the disclosure relates to an engineered cell expressing any one of the engineered receptors or dual receptor systems described herein.

In some embodiments, the engineered cell described herein comprises a polynucleotide encoding an amino acid sequence of any one of SEQ ID NOs: 132-145, or an amino acid sequence that is at least 90%, 91%, 92%, 93%, 94%, 95%, 96%, 97%, 98%, 99% or 100% identical to SEQ ID NOs: 132-145.

In some embodiments, the engineered cell is an immune cell.

In some embodiments, the immune cell is an NK cell or a T cell.

In some embodiments, the engineered cell is a T cell.

In some embodiments, the T cell is selected from the group consisting of cytotoxic T cell, a helper T cell, a natural killer T (NK-T) cell, an αβT cell and a γδT cell.

In one aspect, the disclosure relates to a method for producing an engineered cell, comprising introducing a vector described herein into a cell in vitro or ex vivo.

In some embodiments, the vector is a viral vector and the introducing is carried out by transduction.

In one aspect, the disclosure relates to a method of treating cancer in a subject, comprising administering an effective amount of the antibody or antigen-binding fragment thereof described herein, the antibody-drug conjugate described herein, the pharmaceutical composition described herein, or the engineered cell described herein to the subject.

In some embodiments, the cancer is acute myeloid leukemia (AML), chronic myelogenous leukemia (CML) or myelodysplastic syndromes (MDS).

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope in an antigen. Non-limiting examples of antibodies include: monoclonal antibodies, polyclonal antibodies, multi-specific antibodies (e.g., bi-specific antibodies), single-chain antibodies, single variable domain (VHH) antibodies, chimeric antibodies, human antibodies, and humanized antibodies. In some embodiments, an antibody can contain an Fc region of a human antibody.

The term antibody also includes derivatives, e.g., multi-specific antibodies, bi-specific antibodies, single-chain antibodies, diabodies, and linear antibodies formed from these antibodies or antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain, a variable domain of light chain or a VHH). Non-limiting examples of antibody fragments include, e.g., Fab, Fab′, F(ab′)2, and Fv fragments, scFv, and VHH.

As used herein, the terms “subject” and “patient” are used interchangeably throughout the specification and describe an animal, human or non-human, to whom treatment according to the methods of the present disclosure is provided. Veterinary and non-veterinary applications are contemplated in the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody or an antigen-binding fragment, the phrases “specifically binding” and “specifically binds” mean that the antibody or an antigen-binding fragment interacts with its target molecule preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to CLL1 may be referred to as a CLL1 antibody, a CLL1-specific antibody or an anti-CLL1 antibody.

As used herein, the term “bispecific antibody” refers to an antibody that binds to two different epitopes. The epitopes can be on the same antigen or on different antigens.

As used herein, the term “trispecific antibody” refers to an antibody that binds to three different epitopes. The epitopes can be on the same antigen or on different antigens.

As used herein, the term “multispecific antibody” refers to an antibody that binds to two or more different epitopes. The epitopes can be on the same antigen or on different antigens. A multispecific antibody can be e.g., a bispecific antibody or a trispecific antibody. In some embodiments, the multispecific antibody binds to two, three, four, five, or six different epitopes.

As used herein, a “VHH” refers to the variable domain of a heavy chain only antibody. In some embodiments, the VHH is a humanized VHH.

As used herein, a “chimeric antigen receptor” or “CAR” refers to a fusion protein comprising an extracellular domain capable of binding to an antigen, and an intracellular region comprising one or more intracellular signaling domains derived from signal transducing proteins. These intracellular signaling domains are typically different from the polypeptide from which the extracellular domain is derived. The extracellular domain can be any proteinaceous molecule or part thereof that can specifically bind to a predetermined antigen. In some embodiments, the extracellular domain comprises an antibody or antigen binding fragment thereof. In some embodiments, the intracellular signaling domain can be any oligopeptide or polypeptide domain known to function to transmit a signal causing activation or inhibition of a biological process in a cell, for example, activation of an immune cell such as a T cell or a NK cell.

As used herein, a “tandem CAR” refers to a CAR comprising two or more extracellular domain capable of binding to an antigen. In some embodiments, a tandem CAR can have 2, 3, 4, 5, 6, 7, 8, 9, or 10 extracellular domains that are capable of binding to an antigen. These antigen-binding domains can be the same or different. In some embodiments, they can bind to the same or different antigens. In some embodiments, they can bind to different epitopes on the same antigen.

As used herein, a “dual receptor system” or a “dual CAR system” refers to a system comprising two or more engineered receptors (e.g., CARs), each targeting a different molecule or a different epitope. Each engineered receptor (e.g., CAR) can further include a transmembrane region; and/or an intracellular signaling domain. In some embodiments, the dual CAR system described herein includes two engineered receptors. In some embodiments, the dual CAR system described herein includes a first CAR targeting CLL1 and a second CAR targeting CD33. In some embodiments, the engineered receptors are expressed in a single vector. In some embodiments, the engineered receptors are linked by a self-cleaving peptide (e.g., a P2A protein). In some embodiments, the two engineered receptors are expressed in different vectors. Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the disclosure will be apparent from the following detailed description and figures, and from the claims.

### DESCRIPTION OF DRAWINGS

Exemplary embodiments are illustrated in referenced figures. It is intended that the embodiments and figures disclosed herein are to be considered illustrative rather than restrictive.

FIG. 1A shows the binding of sera from immunized animals at different time points with immobilized human CLL1 using an enzyme-linked immune sorbent assay (ELISA) experiment.

FIG. 1B shows the binding of isolated pre-immune and post-immune immunoglobulins with immobilized human CLL1 using ELISA.

FIG. 2 shows the binding affinity of 13 anti-CLL1 IgG1 antibodies to human CLL1.

FIG. 3 shows the cytotoxicity of anti-CLL1 CAR-T cells against CLL1-HEK293T cells, as compared to BM CAR-T cells.

FIG. 4A shows the cytokine secretion (IFN-γ production) of anti-CLL1 CAR-T cells against CLL1-HEK293T cells compared to that of the BM CAR-T cells.

FIG. 4B shows the cytokine secretion (GM-CSF production) of anti-CLL1 CAR-T cells against CLL1-HEK293T cells compared to that of the BM CAR-T cells.

FIG. 5A shows the killing efficacy of anti-CLL1 CAR-T cells, as marked by residual tumor cells (CFSE-THP-1) in a long-term co-culture assay (repeated tumor stimulation assay) where the tumor cells were co-cultured with anti-CLL1 CAR-T cells.

FIG. 5B shows the T cell proliferation rate in a long-term co-culture assay (repeated tumor stimulation assay) where the tumor cells were co-cultured with anti-CLL1 CAR-T cells.

FIG. 6 shows the in vivo anti-tumor activities of anti-CLL1 CAR-T cells in a U937-Luc xenograft mouse model.

FIG. 7 is a schematic illustration of an example of an anti-CD33/CLL1 tandem CAR that binds to CD33 and CLL1.

FIG. 8 shows the in vitro cytotoxicity of anti-CD33/CLL1 tandem CARs against THP-1 cells, as compared to anti-CLL1 single target CARs.

FIG. 9A shows the killing efficacy of various anti-CD33/CLL1 tandem CAR-T cells in a long-term co-culture assay (repeated tumor stimulation assay) where the tumor cells were co-cultured with anti-CD33/CLL1 tandem CAR-T cells, as compared to anti-CLL1 single target CARs.

FIG. 9B shows the T cell proliferation rate of anti-CD33/CLL1 tandem CARs in a long-term co-culture assay (repeated tumor stimulation assay) where the tumor cells were co-cultured with anti-CD33/CLL1 tandem CAR-T cells, as compared to anti-CLL1 single target CARs.

FIG. 10A shows the cytokine releasing (IFN-γ production) of anti-CD33/CLL1 tandem CAR-T cells, as compared to anti-CLL1 single-target CAR-T cells.

FIG. 10B shows the cytokine releasing (GM-CSF production) of anti-CD33/CLL1 tandem CAR-T cells, as compared to anti-CLL1 single-target CAR-T cells.

FIG. 10C is a schematic diagram of an exemplary dual CAR system.

FIG. 11 shows the in vivo anti-tumor activities of anti-CD33/CLL1 tandem CAR-T cells in a U937-Luc xenograft mouse model.

FIG. 12 shows the anti-tumor activities of dual CAR-T cells assessed using the in vitro LDH (lactate dehydrogenase) assay, as compared to anti-CLL1 single-target CAR-T cells.

FIG. 13 shows the in vivo anti-tumor activities of dual CAR-T cells in a U937-Luc xenograft mouse model, as compared to anti-CLL1 single-target CAR-T cells.

FIG. 14A shows the amino acid sequences of the VL CDRs and VH CDRs of examples of the anti-CLL1 antibodies and antigen-binding fragments described herein.

FIG. 14B shows the amino acid sequences of the VL CDRs and VH CDRs of examples of the anti-CD33 antibodies and antigen-binding fragments described herein.

FIG. 15 shows the amino acid sequence of examples of the single-target CARs, scFvs, VLs, VHs, the signal peptide and the human CLL1 described herein.

### DETAILED DESCRIPTION

Acute myeloid leukemia (AML) is one of the most common types of adult acute leukemia. Standard chemotherapies can induce complete remission in selected patients; however, a majority of patients eventually relapse and succumb to the disease. Thus, the development of novel therapeutics for AML is urgently needed. Human C-type lectin-like molecule-1 (CLL1) is a type II transmembrane glycoprotein, and its expression is restricted to myeloid cells and the majority of AML blasts. Moreover, CLL1 is expressed in leukemia stem cells (LSCs), but absent in hematopoietic stem cells (HSCs), which may provide a potential therapeutic target for AML treatment. Since CLL1 is selectively expressed on myeloid cells, compositions that recognize and bind to CLL1 can be useful for such therapies of hematologic malignancies, especially those of myeloid origin.

The present disclosure provides isolated antibodies or immunologically functional antibody fragments (i.e., antigen binding fragments) thereof that bind CLL1 epitopes with high affinity, which antibodies can be used for treating a variety of diseases in which CLL1 is implicated (e.g., overexpressed), such as hematologic malignancies, including leukemias. In some embodiments, the antibodies or antibody fragments thereof bind to primate and human CLL1. In some embodiments, the antibodies and antigen-binding fragments bind with high affinity to human CLL1.

### CLL1 and Cancer

CLL1 is expressed on 92% AML and is absent on granulocyte-macrophage progenitors (GMPs) (Bakker A B, et al. Cancer Res. 2004; 64:8443-50.) More importantly, CLL1 is also expressed on leukemic stem cell (LSC), which possesses the ability to indefinitely self-renew and produce plenty of daughter blast cells with a specific phenotype of CLL1, CD123, CD44, CD96, CD90, CD32, CD25, and TIM-3, acting as one of most important reasons of leukemia relapse (Siveen K S, et al. Mol Cancer. 2017; 16: 13. Yoshida G J, et al. Cancer Sci. 2016; 107:5-11. Zhou J, et al. World J Stem Cells. 2014; 6: 473-84). Accordingly, its differential characterizations allow for CLL1 to be considered as a target for treatment of AML. CLL1 is also named as c-type lectin domain family 12, member A (CLEC12A), myeloid inhibitory c-type lectin-like receptor (MICL), dendritic cell (DC)-associated C-type lectin 2 (DCAL-2), or CD371.

Non-hematological tissues in human seldom express CLL1/hMICL. In the hematopoietic tree, CLL1 is mainly expressed on almost all the granulocytes and monocytes, approximately 61.8% of granulocyte and monocyte precursors; 41.6% of progenitors and only on 2.5% of HSC defined as CD34+CD38−, but not on T, B, NK cells and erythrocytes and their precursors (Wang J, et al. J Hematol Oncol. 2018; 11: 7).

CLL1 is an excellent therapeutic target for AML due to its overexpression in blasts and leukemic stem cells (LSCs). The present disclosure provides antibodies targeting CLL1, chimeric antigen receptors targeting CLL1, and methods of use thereof.

### Antibodies and Antigen Binding Fragments

The present disclosure provides antibodies and antigen-binding fragments thereof that bind to CLL1. In general, antibodies (also called immunoglobulins) are made up of two classes of polypeptide chains, light chains and heavy chains. A non-limiting antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can have two identical copies of a light chain and two identical copies of a heavy chain. The heavy chains, which each contain one variable domain (or variable region, V_{H}) and multiple constant domains (or constant regions), bind to one another via disulfide bonding within their constant domains to form the “stem” of the antibody. The light chains, which each contain one variable domain (or variable region, V_{L}) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, Antibody engineering, Springer Berlin Heidelberg, 2001. 422-439; Abhinandan, et al., Molecular immunology 45.14 (2008): 3832-3839; Wu, T. T. and Kabat, E. A. (1970) J. Exp. Med. 132: 211-250; Martin et al., Methods Enzymol. 203:121-53 (1991); Morea et al., Biophys Chem. 68(1-3):9-16 (October 1997); Morea et al., J Mol Biol. 275(2):269-94 (January 1998); Chothia et al., Nature 342(6252):877-83 (December 1989); Ponomarenko and Bourne, BMC Structural Biology 7:64 (2007); each of which is incorporated herein by reference in its entirety. In some embodiments, the Kabat definition is used. In some embodiments, the Chothia definition is used. In some embodiments, a combination of Kabat and Chothia, and/or some other definitions that are well known in the art are used.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, Frontiers in immunology 5 (2014); Irani, et al., Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)2, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the antigen binding fragment can form a part of a chimeric antigen receptor (CAR). In some embodiments, the chimeric antigen receptor are fusions of single-chain variable fragments (scFv) as described herein, fused to CD3-zeta transmembrane and endodomain. In some embodiments, the chimeric antigen receptor also comprises intracellular signaling domains from various costimulatory protein receptors (e.g., CD28, 41BB, ICOS). In some embodiments, the chimeric antigen receptor comprises multiple signaling domains, e.g., CD3z-CD28-41BB or CD3z-CD28-OX40, to increase potency. Thus, in one aspect, the disclosure further provides cells (e.g., T cells) that express the chimeric antigen receptors as described herein. In some embodiments, the scFv has one heavy chain variable domain, and one light chain variable domain.

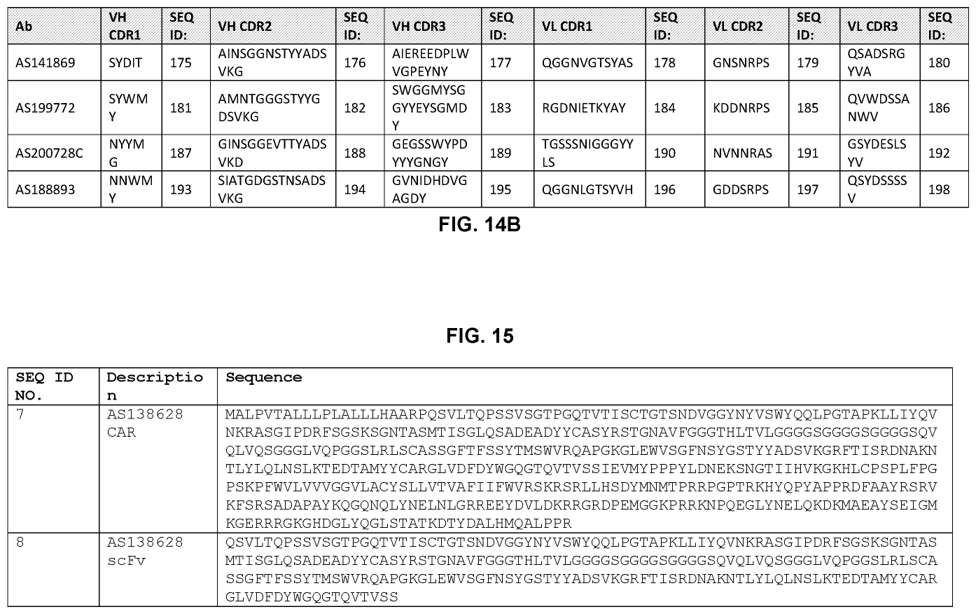

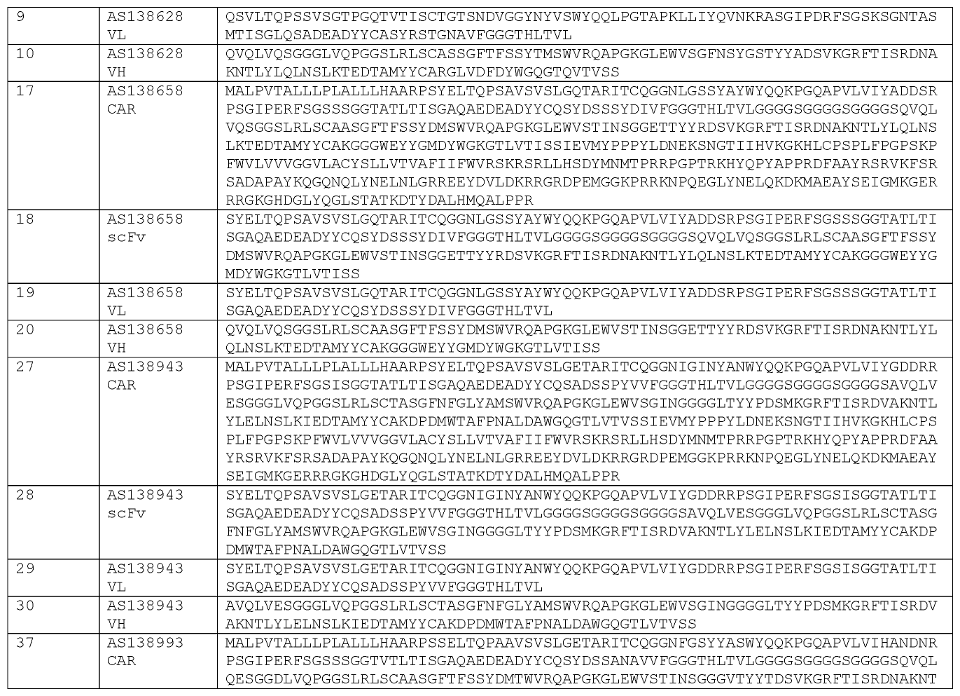

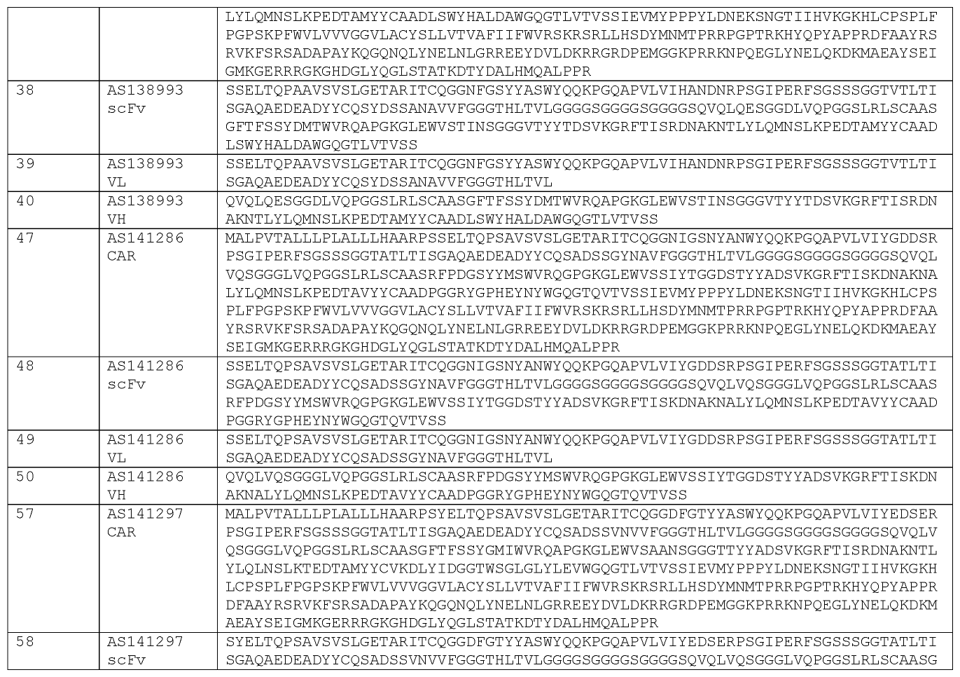

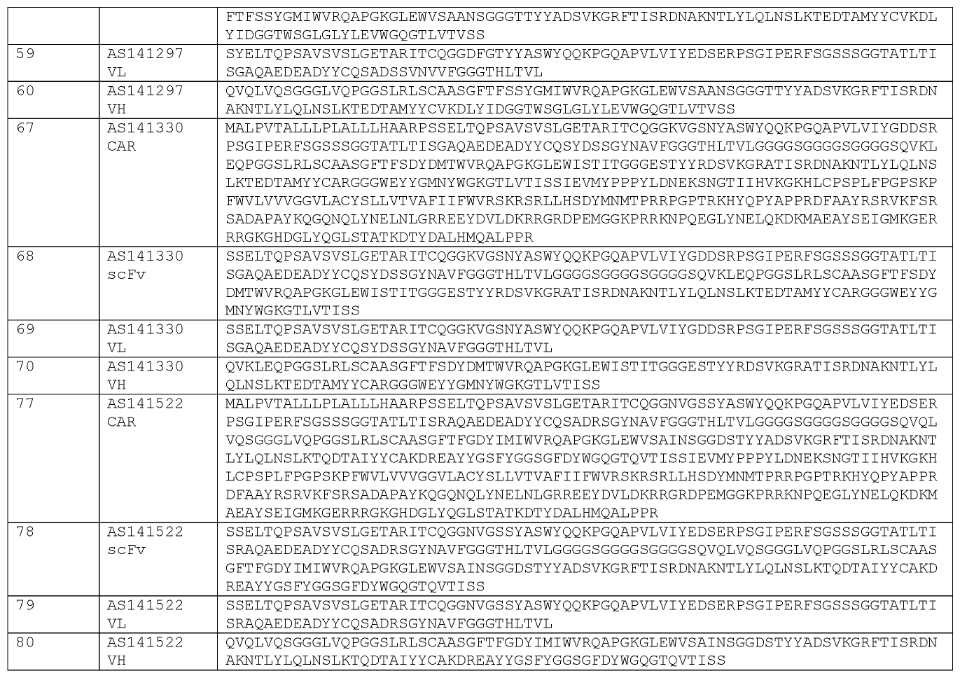

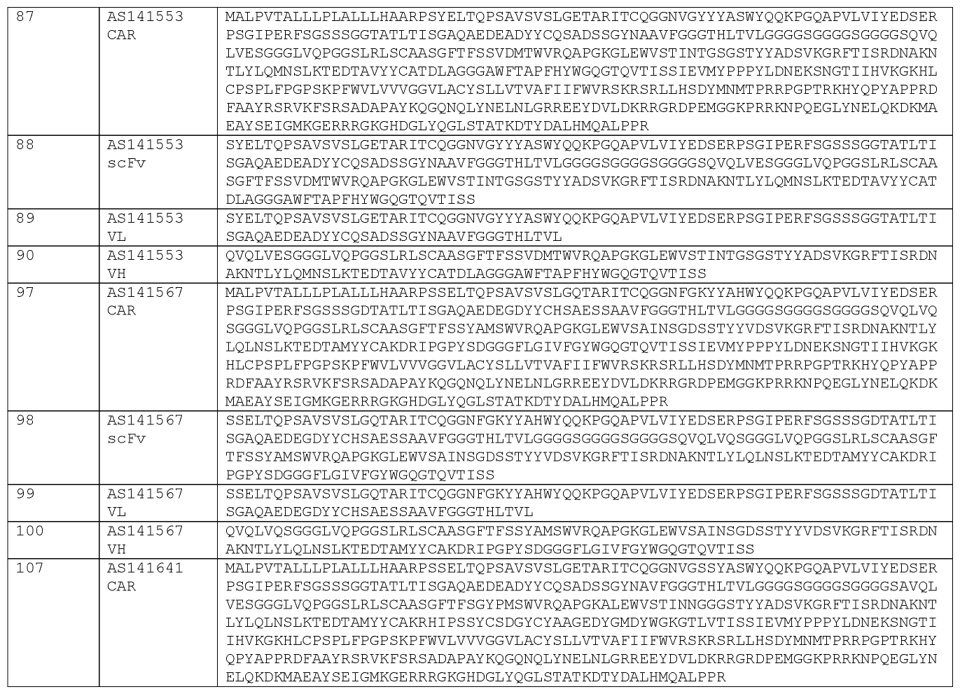

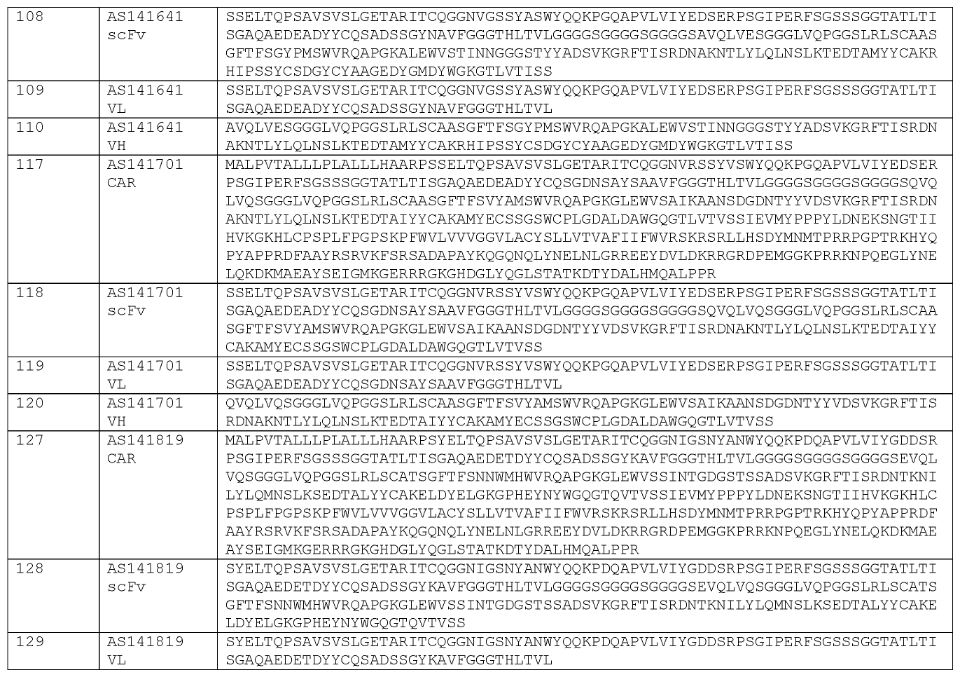

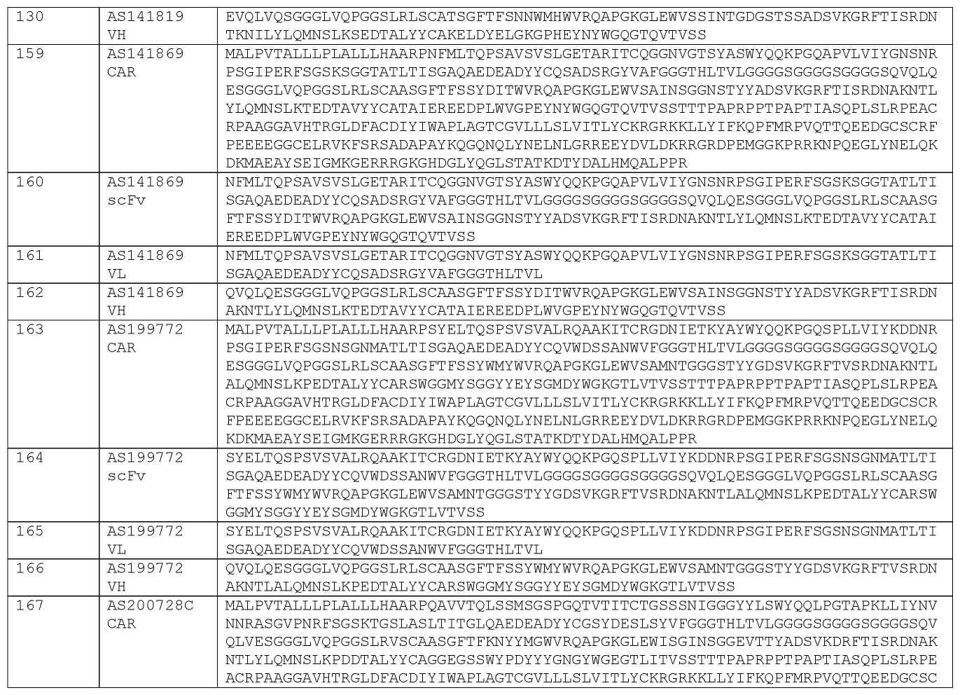

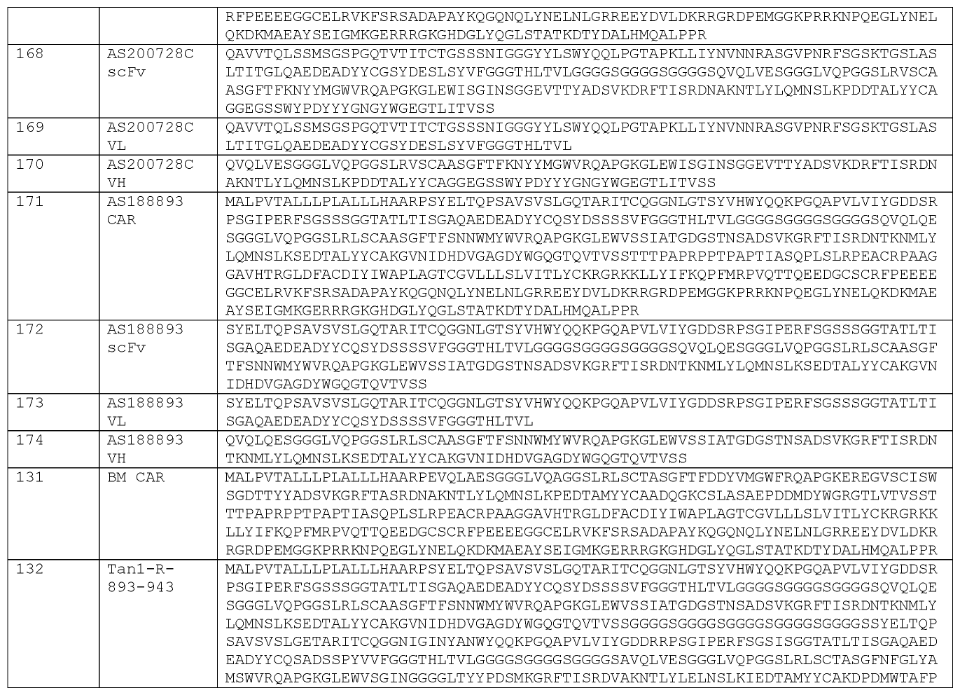

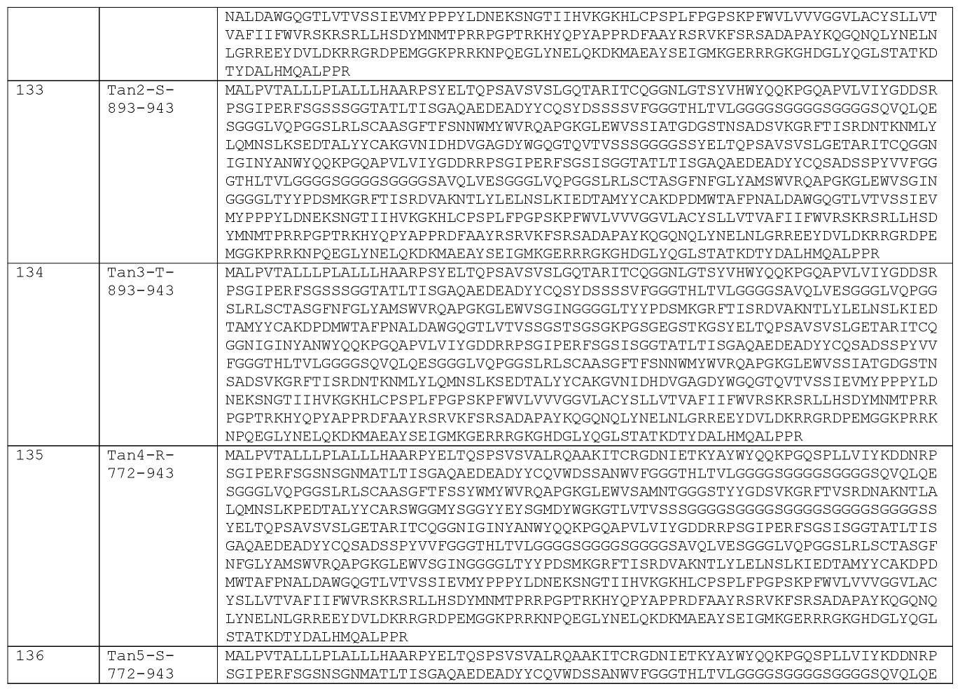

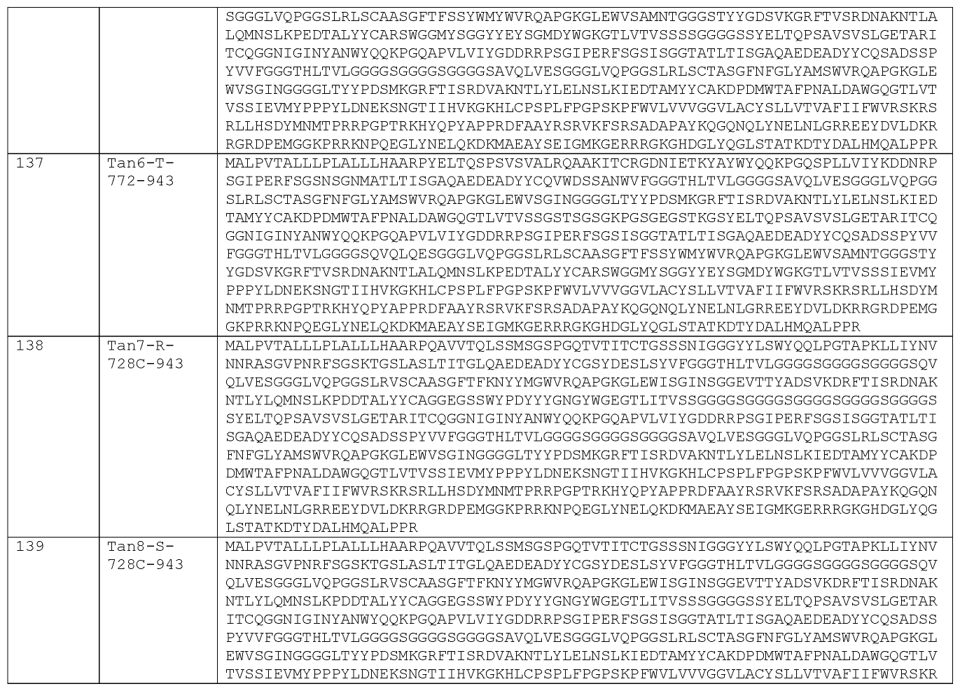

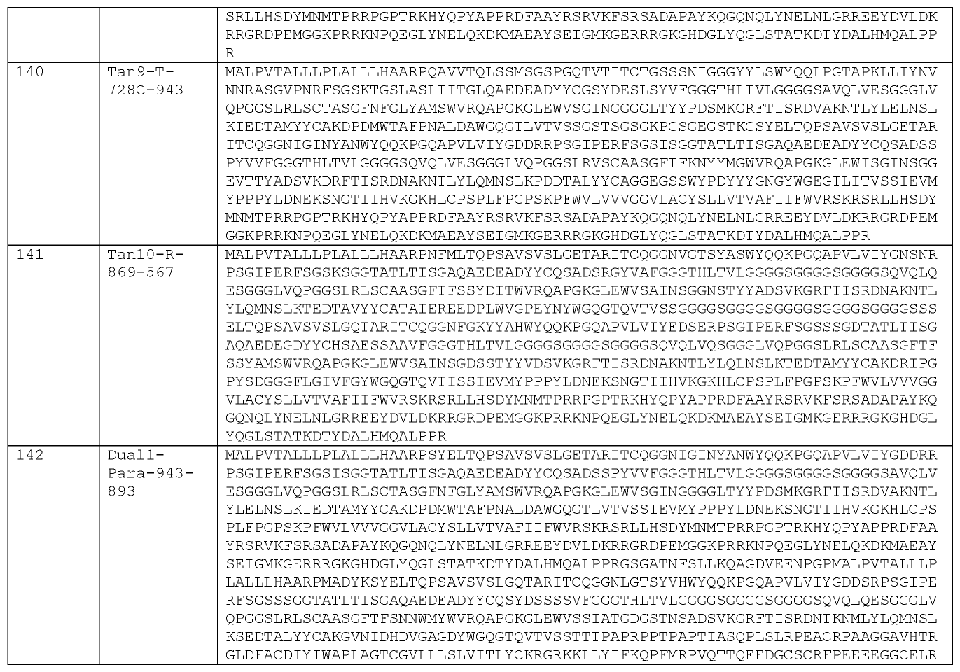

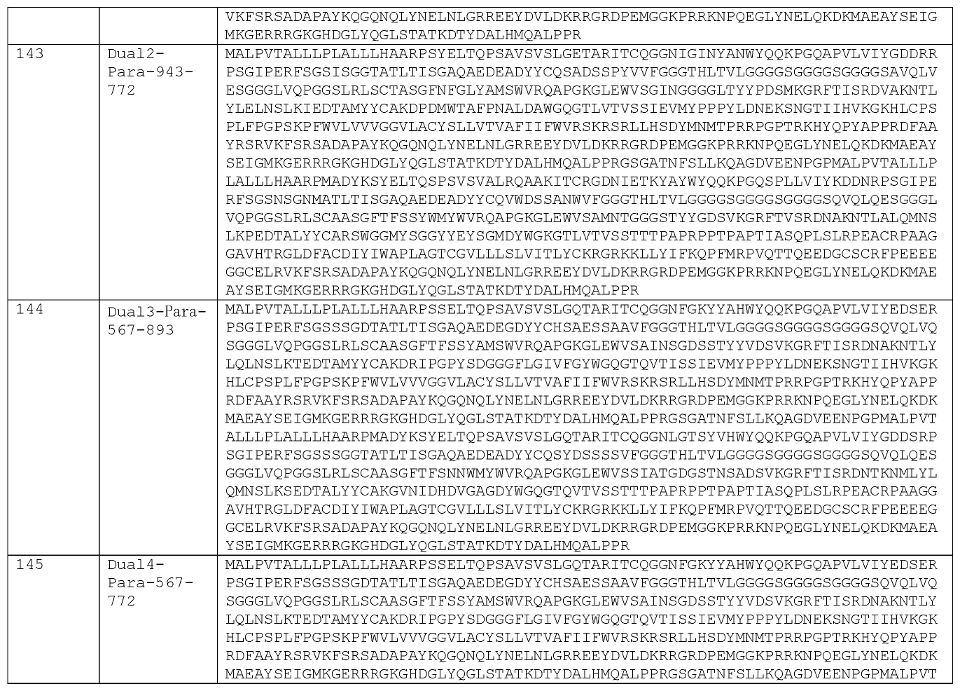

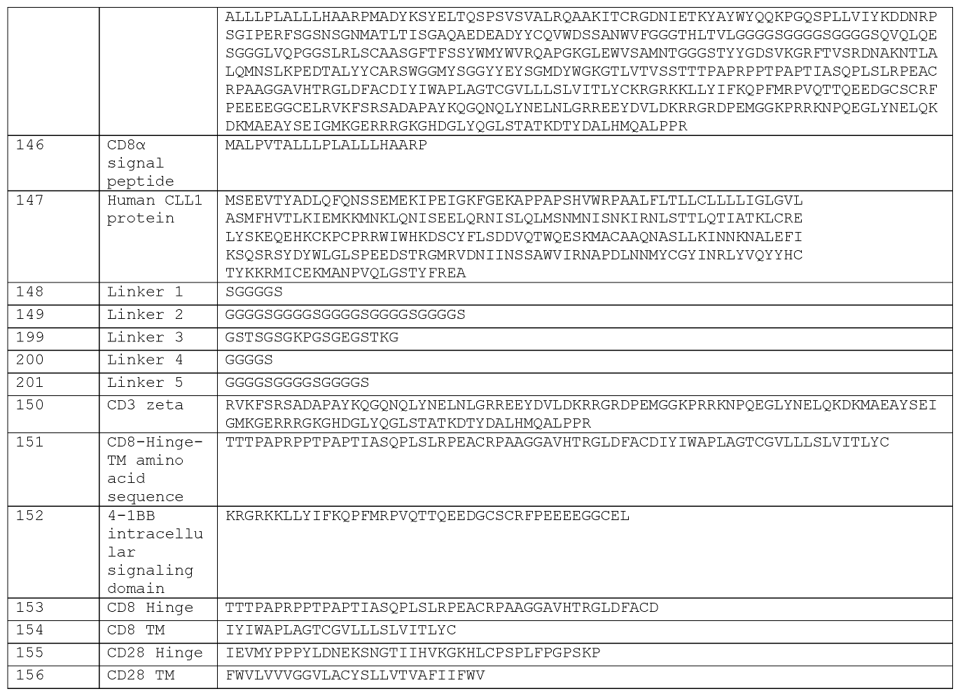

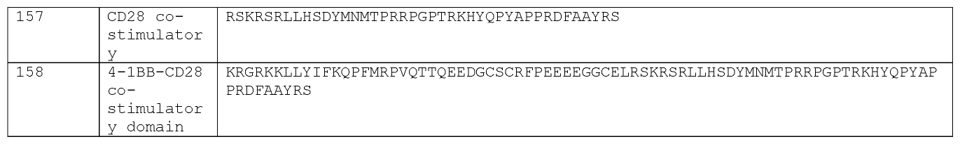

The disclosure provides antibodies and antigen-binding fragments thereof that specifically bind CLL1. The disclosure provides e.g., antibodies and antigen-binding fragments thereof, the chimeric antibodies thereof, and the humanized antibodies thereof (e.g., antibodies as shown in FIGS. 14A and 15). In some embodiments, the present disclosure provides antibodies and antigen-binding fragments thereof of AS138628, AS138658, AS138943, AS138993, AS141286, AS141297, AS141330, AS141522, AS141553, AS141567, AS141641, AS141701, and AS141819.

The CDR sequences for AS138628 include CDRs of the heavy chain variable domain, SEQ ID NOs: 1, 2, and 3, and CDRs of the light chain variable domain, SEQ ID NOs: 4, 5, and 6.

The CDR sequences for AS138658 include CDRs of the heavy chain variable domain, SEQ ID NOs: 11, 12, and 13, and CDRs of the light chain variable domain, SEQ ID NOs: 14, 15, and 16.

The CDR sequences for AS138943 include CDRs of the heavy chain variable domain, SEQ ID NOs: 21, 22, and 23, and CDRs of the light chain variable domain, SEQ ID NOs: 24, 25 and 26.

The CDR sequences for AS138993 include CDRs of the heavy chain variable domain, SEQ ID NOs: 31, 32, and 33, and CDRs of the light chain variable domain, SEQ ID NOs: 34, 35 and 36.

The CDR sequences for AS141286 include CDRs of the heavy chain variable domain, SEQ ID NOs: 41, 42, and 43, and CDRs of the light chain variable domain, SEQ ID NOs: 44, 45 and 46.

The CDR sequences for AS141297 include CDRs of the heavy chain variable domain, SEQ ID NOs: 51, 52, and 53, and CDRs of the light chain variable domain, SEQ ID NOs: 54, 55 and 56.

The CDR sequences for AS141330 include CDRs of the heavy chain variable domain, SEQ ID NOs: 61, 62, and 63, and CDRs of the light chain variable domain, SEQ ID NOs: 64, 65 and 66.

The CDR sequences for AS141522 include CDRs of the heavy chain variable domain, SEQ ID NOs: 71, 72, and 73, and CDRs of the light chain variable domain, SEQ ID NOs: 74, 75 and 76.

The CDR sequences for AS141553 include CDRs of the heavy chain variable domain, SEQ ID NOs: 81, 82, and 83, and CDRs of the light chain variable domain, SEQ ID NOs: 84, 85 and 86.

The CDR sequences for AS141567 include CDRs of the heavy chain variable domain, SEQ ID NOs: 91, 92, and 93, and CDRs of the light chain variable domain, SEQ ID NOs: 94, 95 and 96.

The CDR sequences for AS141641 include CDRs of the heavy chain variable domain, SEQ ID NOs: 101, 102, and 103, and CDRs of the light chain variable domain, SEQ ID NOs: 104, 105, and 106.

The CDR sequences for AS141701 include CDRs of the heavy chain variable domain, SEQ ID NOs: 111, 112, and 113, and CDRs of the light chain variable domain, SEQ ID NOs: 114, 115 and 116.

The CDR sequences for AS141819 include CDRs of the heavy chain variable domain, SEQ ID NOs: 121, 122, and 123, and CDRs of the light chain variable domain, SEQ ID NOs: 124, 125 and 126.

Furthermore, in some embodiments, the antibodies or antigen-binding fragments thereof described herein can also contain one, two, or three heavy chain variable region CDRs selected from VH CDRs in FIGS. 14A-14B, and one, two, or three light chain variable region CDRs selected from VL CDRs in FIGS. 14A-14B. In some embodiments, the VL CDR1, VL CDR2, VL CDR3, VH CDR1, VH CDR2, and VH CDR3 are determined by Kabat definitions.

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, 95%, 96%, 97%, 98%, 99% or 100% identical to a selected VH CDR1 amino acid sequence, the CDR2 region comprises or consists of an amino acid sequence that is at least 80%, 85%, 90%, 95%, 96%, 97%, 98%, 99% or 100% identical to a selected VH CDR2 amino acid sequence, and the CDR3 region comprises or consists of an amino acid sequence that is at least 80%, 85%, 90%, 95%, 96%, 97%, 98%, 99% or 100% identical to a selected VH CDR3 amino acid sequence. In some embodiments, the antibodies can have a light chain variable region (VL) comprising CDRs 1, 2, 3, wherein the CDR1 region comprises or consists of an amino acid sequence that is at least 80%, 85%, 90%, 95%, 96%, 97%, 98%, 99% or 100% identical to a selected VL CDR1 amino acid sequence, the CDR2 region comprises or consists of an amino acid sequence that is at least 80%, 85%, 90%, 95%, 96%, 97%, 98%, 99% or 100% identical to a selected VL CDR2 amino acid sequence, and the CDR3 region comprises or consists of an amino acid sequence that is at least 80%, 85%, 90%, 95%, 96%, 97%, 98%, 99% or 100% identical to a selected VL CDR3 amino acid sequence. The selected VH CDRs 1, 2, 3 amino acid sequences and the selected VL CDRs, 1, 2, 3 amino acid sequences as determined by Kabat are shown in FIGS. 14A-14B.

In some embodiments, the antibody or an antigen-binding fragment described herein can contain a heavy chain variable domain containing one, two, or three of the CDRs in FIGS. 14A-14B with zero, one or two amino acid insertions, deletions, or substitutions in each of the CDRs. In some embodiments, the antibody or an antigen-binding fragment described herein can contain a light chain variable domain containing one, two, or three of the CDRs in FIGS. 14A-14B with zero, one or two amino acid insertions, deletions, or substitutions in each of the CDRs.

The amino acid sequences for heavy chain variable regions and light variable regions of the various antibodies are also provided. As there are different ways to humanize an antibody (e.g., a sequence can be modified with different amino acid substitutions), the heavy chain and the light chain of an antibody can have more than one version of humanized sequences.

The disclosure also provides antibodies or antigen-binding fragments thereof that bind to CLL1. The antibodies or antigen-binding fragments thereof contain a heavy chain variable region (VH) comprising or consisting of an amino acid sequence that is at least 80%, 85%, 90%, 95%, 96%, 97%, 98% or 99% identical to a selected VH sequence, and a light chain variable region (VL) comprising or consisting of an amino acid sequence that is at least 80%, 85%, 90%, 95%, 96%, 97%, 98% or 99% identical to a selected VL sequence. In some embodiments, the selected VH sequence and the selected VL sequences are derived from AS138628, AS138658, AS138943, AS138993, AS141286, AS141297, AS141330, AS141522, AS141553, AS141567, AS141641, AS141701, and AS141819.

The amino acid sequence for the heavy chain variable region of antibody AS138628 is set forth in SEQ ID NO: 10. The amino acid sequence for the light chain variable region of AS138628 antibody is set forth in SEQ ID NO: 9.

The amino acid sequence for the heavy chain variable region of antibody AS138658 is set forth in SEQ ID NO: 20. The amino acid sequence for the light chain variable region of AS138658 antibody is set forth in SEQ ID NO: 19.

The amino acid sequence for the heavy chain variable region of antibody AS138943 is set forth in SEQ ID NO: 30. The amino acid sequence for the light chain variable region of AS138943 antibody is set forth in SEQ ID NO: 29.

The amino acid sequence for the heavy chain variable region of antibody AS138993 is set forth in SEQ ID NO: 40. The amino acid sequence for the light chain variable region of AS138993 antibody is set forth in SEQ ID NO: 39.

The amino acid sequence for the heavy chain variable region of antibody AS141286 is set forth in SEQ ID NO: 50. The amino acid sequence for the light chain variable region of AS141286 antibody is set forth in SEQ ID NO: 49.

The amino acid sequence for the heavy chain variable region of antibody AS141297 is set forth in SEQ ID NO: 60. The amino acid sequence for the light chain variable region of AS141297 antibody is set forth in SEQ ID NO: 59.

The amino acid sequence for the heavy chain variable region of antibody AS141330 is set forth in SEQ ID NO: 70. The amino acid sequence for the light chain variable region of AS141330 antibody is set forth in SEQ ID NO: 69.

The amino acid sequence for the heavy chain variable region of antibody AS141522 is set forth in SEQ ID NO: 80. The amino acid sequence for the light chain variable region of AS141522 antibody is set forth in SEQ ID NO: 79.

The amino acid sequence for the heavy chain variable region of antibody AS141553 is set forth in SEQ ID NO: 90. The amino acid sequence for the light chain variable region of AS141553 antibody is set forth in SEQ ID NO: 89.

The amino acid sequence for the heavy chain variable region of antibody AS141567 is set forth in SEQ ID NO: 100. The amino acid sequence for the light chain variable region of AS141567 antibody is set forth in SEQ ID NO: 99.

The amino acid sequence for the heavy chain variable region of antibody AS141641 is set forth in SEQ ID NO: 110. The amino acid sequence for the light chain variable region of AS141641 antibody is set forth in SEQ ID NO: 109.

The amino acid sequence for the heavy chain variable region of antibody AS141701 is set forth in SEQ ID NO: 120. The amino acid sequence for the light chain variable region of AS141701 antibody is set forth in SEQ ID NO: 119.

The amino acid sequence for the heavy chain variable region of antibody AS141819 is set forth in SEQ ID NO: 130. The amino acid sequence for the light chain variable region of AS141819 antibody is set forth in SEQ ID NO: 129.

The present disclosure also provides humanized antibodies or antigen binding fragments thereof. Humanization percentage means the percentage identity of the heavy chain or light chain variable region sequence as compared to human antibody sequences in International Immunogenetics Information System (IMGT) database. In some embodiments, humanization percentage is greater than 80%, 81%, 82%, 83%, 84%, 85%, 86%, 87%, 88%, 89%, 90%, 91%, 92%, 93%, 94%, or 95%. A detailed description regarding how to determine humanization percentage and how to determine top hits is known in the art, and is described, e.g., in Jones, Tim D., et al, MAbs. Vol. 8. No. 1. Taylor & Francis, 2016, which is incorporated herein by reference in its entirety. A high humanization percentage often has various advantages, e.g., more safe and more effective in humans, more likely to be tolerated by a human subject, and/or less likely to have side effects.

Furthermore, in some embodiments, the antibodies or antigen-binding fragments thereof described herein can also contain one, two, or three heavy chain variable region CDRs (in any order) selected from the groups of SEQ ID NOs for each antibody or antigen-binding fragment listed in FIGS. 14A-14B and 15, and/or one, two, or three light chain variable region CDRs (in any order) selected from the groups of SEQ ID NOs for each antibody or antigen-binding fragment listed in FIGS. 14A-14B and 15. In some embodiments, the antibody or an antigen-binding fragment described herein can contain a heavy chain variable domain containing one, two, or three of the CDRs of any one of the heavy chain CDRs of the antibodies or antigen-binding fragments thereof described herein with zero, one or two amino acid insertions, deletions, or substitutions. In some embodiments, the antibody or an antigen-binding fragment described herein can contain a light chain variable domain containing one, two, or three of the CDRs of any one of the light chain CDRs of the antibodies or antigen-binding fragments thereof described herein with zero, one or two amino acid insertions, deletions, or substitutions. The insertions, deletions, and substitutions can be within the CDR sequence, or at one or both terminal ends of the CDR sequence.

The disclosure also provides antibodies or antigen-binding fragments thereof that bind to CLL1. The antibodies or antigen-binding fragments thereof contain a heavy chain variable region (VH) comprising or consisting of an amino acid sequence that is at least 80%, 85%, 90%, or 95% identical to a selected VH sequence or the VH of a selected scFv, and a light chain variable region (VL) comprising or consisting of an amino acid sequence that is at least 80%, 85%, 90%, or 95% identical to a selected VL sequence or the VL of a selected scFv.

In some embodiments, the selected VH sequence is SEQ ID NO: 10, and the selected VL sequence is SEQ ID NO: 9. In some embodiments, the selected scFv is SEQ ID NO: 8.

In some embodiments, the selected VH sequence is SEQ ID NO: 20, and the selected VL sequence is SEQ ID NO: 19. In some embodiments, the selected scFv is SEQ ID NO: 18.

In some embodiments, the selected VH sequence is SEQ ID NO: 30, and the selected VL sequence is SEQ ID NO: 29. In some embodiments, the selected scFv is SEQ ID NO: 28.

In some embodiments, the selected VH sequence is SEQ ID NO: 40, and the selected VL sequence is SEQ ID NO: 39. In some embodiments, the selected scFv is SEQ ID NO: 38.

In some embodiments, the selected VH sequence is SEQ ID NO: 50, and the selected VL sequence is SEQ ID NO: 49. In some embodiments, the selected scFv is SEQ ID NO: 48.

In some embodiments, the selected VH sequence is SEQ ID NO: 60, and the selected VL sequence is SEQ ID NO: 59. In some embodiments, the selected scFv is SEQ ID NO: 58.

In some embodiments, the selected VH sequence is SEQ ID NO: 70, and the selected VL sequence is SEQ ID NO: 69. In some embodiments, the selected scFv is SEQ ID NO: 68.

In some embodiments, the selected VH sequence is SEQ ID NO: 80, and the selected VL sequence is SEQ ID NO: 79. In some embodiments, the selected scFv is SEQ ID NO: 78.

In some embodiments, the selected VH sequence is SEQ ID NO: 90, and the selected VL sequence is SEQ ID NO: 89. In some embodiments, the selected scFv is SEQ ID NO: 88.

In some embodiments, the selected VH sequence is SEQ ID NO: 100, and the selected VL sequence is SEQ ID NO: 99. In some embodiments, the selected scFv is SEQ ID NO: 98.

In some embodiments, the selected VH sequence is SEQ ID NO: 110, and the selected VL sequence is SEQ ID NO: 109. In some embodiments, the selected scFv is SEQ ID NO: 108.

In some embodiments, the selected VH sequence is SEQ ID NO: 120, and the selected VL sequence is SEQ ID NO: 119. In some embodiments, the selected scFv is SEQ ID NO: 118.

In some embodiments, the selected VH sequence is SEQ ID NO: 130, and the selected VL sequence is SEQ ID NO: 129. In some embodiments, the selected scFv is SEQ ID NO: 128.

The disclosure also provides nucleic acid comprising a polynucleotide encoding a polypeptide comprising an immunoglobulin heavy chain or an immunoglobulin light chain. The immunoglobulin heavy chain or immunoglobulin light chain comprises CDRs of any one of the antibodies or antigen binding fragments thereof described herein, or have sequences of the immunoglobulin heavy chain or immunoglobulin light chain of any one of the antibodies or antigen binding fragments thereof described herein. When the polypeptides are paired with corresponding polypeptide (e.g., a corresponding heavy chain variable region or a corresponding light chain variable region), the paired polypeptides bind to CLL1.

In some embodiments, the nucleic acid described herein comprises a polynucleotide encoding a polypeptide comprising an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 1, 2, and 3, respectively, and wherein the VH, when paired with a light chain variable region (VL) comprising the amino acid sequence set forth in SEQ ID NO: 4, 5, or 6 binds to CLL1.

In some embodiments, the nucleic acid described herein comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 4, 5, and 6, respectively, and wherein the VL, when paired with a VH comprising the amino acid sequence set forth in SEQ ID NO: 1, 2, or 3 binds to CLL1.

In some embodiments, the nucleic acid described herein comprises a polynucleotide encoding a polypeptide comprising an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 11, 12, and 13, respectively, and wherein the VH, when paired with a light chain variable region (VL) comprising the amino acid sequence set forth in SEQ ID NO: 14, 15, or 16 binds to CLL1.

In some embodiments, the nucleic acid described herein comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 14, 15, and 16, respectively, and wherein the VL, when paired with a VH comprising the amino acid sequence set forth in SEQ ID NO: 11, 12, or 13 binds to CLL1.

In some embodiments, the nucleic acid described herein comprises a polynucleotide encoding a polypeptide comprising an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 21, 22, and 23, respectively, and wherein the VH, when paired with a light chain variable region (VL) comprising the amino acid sequence set forth in SEQ ID NO: 24, 25, or 26 binds to CLL1.

In some embodiments, the nucleic acid described herein comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 24, 25, and 26, respectively, and wherein the VL, when paired with a VH comprising the amino acid sequence set forth in SEQ ID NO: 21, 22, or 23 binds to CLL1.

In some embodiments, the nucleic acid described herein comprises a polynucleotide encoding a polypeptide comprising an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 31, 32, and 33, respectively, and wherein the VH, when paired with a light chain variable region (VL) comprising the amino acid sequence set forth in SEQ ID NO: 34, 35, or 36 binds to CLL1.

In some embodiments, the nucleic acid described herein comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 34, 35, and 36, respectively, and wherein the VL, when paired with a VH comprising the amino acid sequence set forth in SEQ ID NO: 31, 32, or 33 binds to CLL1.

In some embodiments, the nucleic acid described herein comprises a polynucleotide encoding a polypeptide comprising an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 41, 42, and 43, respectively, and wherein the VH, when paired with a light chain variable region (VL) comprising the amino acid sequence set forth in SEQ ID NO: 44, 45, or 46 binds to CLL1.

In some embodiments, the nucleic acid described herein comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 44, 45, and 46, respectively, and wherein the VL, when paired with a VH comprising the amino acid sequence set forth in SEQ ID NO: 41, 42, or 43 binds to CLL1.

In some embodiments, the nucleic acid described herein comprises a polynucleotide encoding a polypeptide comprising an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 51, 52, and 53, respectively, and wherein the VH, when paired with a light chain variable region (VL) comprising the amino acid sequence set forth in SEQ ID NO: 54, 55, or 56 binds to CLL1.

In some embodiments, the nucleic acid described herein comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 54, 55, and 56, respectively, and wherein the VL, when paired with a VH comprising the amino acid sequence set forth in SEQ ID NO: 51, 52, or 53 binds to CLL1.

In some embodiments, the nucleic acid described herein comprises a polynucleotide encoding a polypeptide comprising an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 61, 62, and 63, respectively, and wherein the VH, when paired with a light chain variable region (VL) comprising the amino acid sequence set forth in SEQ ID NO: 64, 65, or 66 binds to CLL1.

In some embodiments, the nucleic acid described herein comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 64, 65, and 66, respectively, and wherein the VL, when paired with a VH comprising the amino acid sequence set forth in SEQ ID NO: 61, 62, or 63 binds to CLL1.

In some embodiments, the nucleic acid described herein comprises a polynucleotide encoding a polypeptide comprising an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 71, 72, and 73, respectively, and wherein the VH, when paired with a light chain variable region (VL) comprising the amino acid sequence set forth in SEQ ID NO: 74, 75, or 76 binds to CLL1.

In some embodiments, the nucleic acid described herein comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 74, 75, and 76, respectively, and wherein the VL, when paired with a VH comprising the amino acid sequence set forth in SEQ ID NO: 71, 72, or 73 binds to CLL1.

In some embodiments, the nucleic acid described herein comprises a polynucleotide encoding a polypeptide comprising an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 81, 82, and 83, respectively, and wherein the VH, when paired with a light chain variable region (VL) comprising the amino acid sequence set forth in SEQ ID NO: 84, 85, or 86 binds to CLL1.

In some embodiments, the nucleic acid described herein comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 84, 85, and 86, respectively, and wherein the VL, when paired with a VH comprising the amino acid sequence set forth in SEQ ID NO: 81, 82, or 83 binds to CLL1.

In some embodiments, the nucleic acid described herein comprises a polynucleotide encoding a polypeptide comprising an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 91, 92, and 93, respectively, and wherein the VH, when paired with a light chain variable region (VL) comprising the amino acid sequence set forth in SEQ ID NO: 94, 95, or 96 binds to CLL1.

In some embodiments, the nucleic acid described herein comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 94, 95, and 96, respectively, and wherein the VL, when paired with a VH comprising the amino acid sequence set forth in SEQ ID NO: 91, 92, or 93 binds to CLL1.

In some embodiments, the nucleic acid described herein comprises a polynucleotide encoding a polypeptide comprising an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 101, 102, and 103, respectively, and wherein the VH, when paired with a light chain variable region (VL) comprising the amino acid sequence set forth in SEQ ID NO: 104, 105, or 106 binds to CLL1.

In some embodiments, the nucleic acid described herein comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 104, 105, and 106, respectively, and wherein the VL, when paired with a VH comprising the amino acid sequence set forth in SEQ ID NO: 101, 102, or 103 binds to CLL1.

In some embodiments, the nucleic acid described herein comprises a polynucleotide encoding a polypeptide comprising an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 111, 112, and 113, respectively, and wherein the VH, when paired with a light chain variable region (VL) comprising the amino acid sequence set forth in SEQ ID NO: 114, 115, or 116 binds to CLL1.

In some embodiments, the nucleic acid described herein comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 114, 115, and 116, respectively, and wherein the VL, when paired with a VH comprising the amino acid sequence set forth in SEQ ID NO: 111, 112, or 113 binds to CLL1.

In some embodiments, the nucleic acid described herein comprises a polynucleotide encoding a polypeptide comprising an immunoglobulin heavy chain or a fragment thereof comprising a heavy chain variable region (VH) comprising complementarity determining regions (CDRs) 121, 122, and 123 comprising the amino acid sequences set forth in SEQ ID NOs: 1, 2, and 3, respectively, and wherein the VH, when paired with a light chain variable region (VL) comprising the amino acid sequence set forth in SEQ ID NO: 124, 125, or 126 binds to CLL1.

In some embodiments, the nucleic acid described herein comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 124, 125, and 126, respectively, and wherein the VL, when paired with a VH comprising the amino acid sequence set forth in SEQ ID NO: 121, 122, or 123 binds to CLL1.

The disclosure provides antibodies and antigen-binding fragments thereof that specifically bind CD33. The disclosure provides e.g., antibodies and antigen-binding fragments thereof, the chimeric antibodies thereof, and the humanized antibodies thereof (e.g., antibodies as shown in FIGS. 14B and 15). In some embodiments, the present disclosure provides antibodies and antigen-binding fragments thereof of AS141869, AS199772, AS200728C and AS188893.

The CDR sequences for AS141869 include CDRs of the heavy chain variable domain, SEQ ID NOs: 175, 176, and 177, and CDRs of the light chain variable domain, SEQ ID NOs: 178, 179 and 180.

The CDR sequences for AS199772 include CDRs of the heavy chain variable domain, SEQ ID NOs: 181, 182, and 183, and CDRs of the light chain variable domain, SEQ ID NOs: 184, 185 and 186.

The CDR sequences for AS200728C include CDRs of the heavy chain variable domain, SEQ ID NOs: 187, 188, and 189, and CDRs of the light chain variable domain, SEQ ID NOs: 190, 191 and 192.

The CDR sequences for AS188893 include CDRs of the heavy chain variable domain, SEQ ID NOs: 193, 194 and 195, and CDRs of the light chain variable domain, SEQ ID NOs: 196, 197 and 198.

The disclosure also provides antibodies or antigen-binding fragments thereof that bind to CD33. The antibodies or antigen-binding fragments thereof contain a heavy chain variable region (VH) comprising or consisting of an amino acid sequence that is at least 80%, 85%, 90%, 95%, 96%, 97%, 98% or 99% identical to a selected VH sequence, and a light chain variable region (VL) comprising or consisting of an amino acid sequence that is at least 80%, 85%, 90%, 95%, 96%, 97%, 98% or 99% identical to a selected VL sequence. In some embodiments, the selected VH sequence and the selected VL sequences are derived from AS141869, AS199772, AS200728C and AS188893.

The amino acid sequence for the heavy chain variable region of antibody AS141869 is set forth in SEQ ID NO: 162. The amino acid sequence for the light chain variable region of AS141869 antibody is set forth in SEQ ID NO: 161.

The amino acid sequence for the heavy chain variable region of antibody AS199772 is set forth in SEQ ID NO: 166. The amino acid sequence for the light chain variable region of AS199772 antibody is set forth in SEQ ID NO: 165.

The amino acid sequence for the heavy chain variable region of antibody AS200728C is set forth in SEQ ID NO: 170. The amino acid sequence for the light chain variable region of AS200728C antibody is set forth in SEQ ID NO: 169.

The amino acid sequence for the heavy chain variable region of antibody AS188893 is set forth in SEQ ID NO: 174. The amino acid sequence for the light chain variable region of AS188893 antibody is set forth in SEQ ID NO: 173.

The disclosure also provides antibodies or antigen-binding fragments thereof that bind to CD33. The antibodies or antigen-binding fragments thereof contain a heavy chain variable region (VH) comprising or consisting of an amino acid sequence that is at least 80%, 85%, 90%, or 95% identical to a selected VH sequence or the VH of a selected scFv, and a light chain variable region (VL) comprising or consisting of an amino acid sequence that is at least 80%, 85%, 90%, or 95% identical to a selected VL sequence or the VL of a selected scFv. In some embodiments, the selected VH sequence is SEQ ID NO: 162, and the selected VL sequence is SEQ ID NO: 161. In some embodiments, the selected scFv is SEQ ID NO: 160. In some embodiments, the selected VH sequence is SEQ ID NO: 166, and the selected VL sequence is SEQ ID NO: 165. In some embodiments, the selected scFv is SEQ ID NO: 164. In some embodiments, the selected VH sequence is SEQ ID NO: 170, and the selected VL sequence is SEQ ID NO: 169. In some embodiments, the selected scFv is SEQ ID NO: 168. In some embodiments, the selected VH sequence is SEQ ID NO: 174, and the selected VL sequence is SEQ ID NO: 173. In some embodiments, the selected scFv is SEQ ID NO: 172. In some embodiments, the immunoglobulin heavy chain or immunoglobulin light chain comprises CDRs of any one of the antibodies or antigen binding fragments thereof described herein.

The disclosure also provides nucleic acid comprising a polynucleotide encoding a polypeptide comprising an immunoglobulin heavy chain or an immunoglobulin light chain. The immunoglobulin heavy chain or immunoglobulin light chain comprises CDRs of any one of the antibodies or antigen binding fragments thereof described herein, or have sequences of the immunoglobulin heavy chain or immunoglobulin light chain of any one of the antibodies or antigen binding fragments thereof described herein. When the polypeptides are paired with corresponding polypeptide (e.g., a corresponding heavy chain variable region or a corresponding light chain variable region), the paired polypeptides bind to CD33.

In some embodiments, the nucleic acid described herein comprises a polynucleotide encoding a polypeptide comprising an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 175, 176, and 177, respectively, and wherein the VH, when paired with a light chain variable region (VL) comprising the amino acid sequence set forth in SEQ ID NO: 178, 179, and 180 binds to CD33.

In some embodiments, the nucleic acid described herein comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 178, 179, and 180, respectively, and wherein the VL, when paired with a VH comprising the amino acid sequence set forth in SEQ ID NO: 175, 176, and 177 binds to CD33.

In some embodiments, the nucleic acid described herein comprises a polynucleotide encoding a polypeptide comprising an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 181, 182, and 183, respectively, and wherein the VH, when paired with a light chain variable region (VL) comprising the amino acid sequence set forth in SEQ ID NO: 184, 185, and 186 binds to CD33.

In some embodiments, the nucleic acid described herein comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 184, 185, and 186, respectively, and wherein the VL, when paired with a VH comprising the amino acid sequence set forth in SEQ ID NO: 181, 182, and 183 binds to CD33.

In some embodiments, the nucleic acid described herein comprises a polynucleotide encoding a polypeptide comprising an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 187, 188, and 189, respectively, and wherein the VH, when paired with a light chain variable region (VL) comprising the amino acid sequence set forth in SEQ ID NO: 190, 191, and 192 binds to CD33.

In some embodiments, the nucleic acid described herein comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 190, 191, and 192, respectively, and wherein the VL, when paired with a VH comprising the amino acid sequence set forth in SEQ ID NO: 187, 188, and 189 binds to CD33.

In some embodiments, the nucleic acid described herein comprises a polynucleotide encoding a polypeptide comprising an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 193, 194, and 195, respectively, and wherein the VH, when paired with a light chain variable region (VL) comprising the amino acid sequence set forth in SEQ ID NO: 196, 197, and 198 binds to CD33. In some embodiments, the nucleic acid described herein comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 196, 197, and 198, respectively, and wherein the VL, when paired with a VH comprising the amino acid sequence set forth in SEQ ID NO: 193, 194, and 195 binds to CD33.

### Chimeric Antigen Receptors and Binding Molecules

Chimeric antigen receptors (CARs) combine many facets of normal T cell activation into a single protein. They link an extracellular antigen recognition domain to an intracellular signaling domain, which activates the T cell when an antigen is bound. CARs typically have the following regions: an antigen binding domain, an extracellular hinge region, a transmembrane region, and an intracellular region. In some embodiments, the intracellular region comprises an intracellular signaling domain or an intracellular signaling region. CARs can be expressed on the surface of T cells using gene transfection techniques. Upon binding to the target tumor antigen, the CARs can activate the T cells to launch specific anti-tumor response in an antigen-dependent manner without being limited by the availability of major histocompatibility complexes (MHC) specific to the target tumor antigen.

The antigen binding domain is exposed to the outside of the cell, in the ectodomain portion of the receptor. It interacts with potential target molecules and is responsible for targeting the CAR-T cell to any cell expressing a matching molecule. The antigen binding domain is typically derived from the variable regions of a monoclonal antibody linked together as a single-chain variable fragment (scFv). An scFv is a chimeric protein made up of the light (VL) and heavy (VH) chains of immunoglobulins, connected with a short linker peptide.

In some embodiments, the linker peptide comprises at least or about 5, 6, 7, 8, 9, 10, 11, 12, 13, 14, 15, 16, 17, 18, 19, 20, 30, 40, or 50 amino acid residues. In some embodiments, the linker peptide comprises at least or about 4, 5, 6, 7, 8, 9, 10, 11, 12, 13, 14, 15, 16, 20, 25, 30, or 40 glycine residues. In some embodiments, the linker peptide comprises at least or about 1, 2, 3, 4, 5, 6, 7, or 8 serine residues. In some embodiments, the linker peptide comprises or consists of both glycine and serine residues. In some embodiments, the linker peptide comprises or consists of a sequence that is at least or about 70%, at least or about 75%, at least or about 80%, at least or about 85%, at least or about 90%, at least or about 95%, at least or about 99%, or 100% identical to GGGGS (SEQ ID NO: 200) or GGGGSGGGGSGGGGS (SEQ ID NO: 201). In some embodiments, the linker sequence comprises at least 1, 2, 3, 4, 5, 6, 7, or 8 repeats of GGGGS (SEQ ID NO: 200). In some embodiments, the linker sequence has no more than 10, 11, 12, 13, 14, 15, 16, 17, 18, 19, 20, 30, 40, or 50 amino acid residues. In some embodiments, the linker peptide comprises 1, 2, 3, 4, or 5 amino acid insertions, deletions, or substitutions.

In some embodiments, the antigen binding domain specifically binds to CLL1 (e.g., human CLL1, or monkey (cynomolgus) CLL1). In some embodiments, the antigen binding domain specifically binds to the extracellular domain (ECD) of CLL1.

The hinge, also called a spacer, is a small structural domain that sits between the antigen binding domain and the cell's outer membrane. An ideal hinge enhances the flexibility of the antigen binding domain, reducing the spatial constraints between the CAR and its target antigen. This promotes antigen binding and synapse formation between the CAR-T cells and target cells. Hinge sequences are often based on IgG hinge regions, or membrane-proximal regions from immune molecules including e.g., CD8, and CD28. In some embodiments, the hinge region is derived from CD8 and/or CD28, and comprises an amino acid sequence that is at least 80%, at least 85%, at least 90%, at least 95%, or 100% identical to SEQ ID NO: 153 or 155.

The transmembrane region is a structural component, consisting of a hydrophobic alpha helix that spans the cell membrane. It anchors the CAR to the plasma membrane, bridging the extracellular hinge and antigen binding domains with the intracellular signaling domain. This domain is essential for the stability of the receptor as a whole. Generally, the transmembrane domain from the most membrane-proximal component of the endodomain is used, but different transmembrane domains result in different receptor stability. The CD28 transmembrane domain is known to result in a highly expressed, stable receptor. In some embodiments, the transmembrane region is derived from CD8 and/or CD28, and comprises an amino acid sequence that is at least 80%, at least 85%, at least 90%, at least 95%, or 100% identical to SEQ ID NO: 154 or 156.

The intracellular T cell signaling region lies in the receptor's endodomain, inside the cell. After an antigen is bound to the external antigen binding domain, CAR receptors cluster together and transmit an activation signal. Then the internal cytoplasmic end of the receptor perpetuates signaling inside the T cell. Normal T cell activation relies on the phosphorylation of immunoreceptor tyrosine-based activation motifs (ITAMs) present in the cytoplasmic domain of CD3-zeta. To mimic this process, CD3-zeta's cytoplasmic domain is commonly used as the main CAR endodomain component. T cells also require co-stimulatory molecules in addition to CD3 signaling in order to persist after activation. For this reason, the endodomains of CAR receptors typically also include one or more chimeric domains from co-stimulatory proteins. Signaling domains from a wide variety of co-stimulatory molecules have been successfully tested, including CD28, CD27, CD134 (OX40), ICOS, hematopoietic cell signal transducer (DAP10) and/or CD137 (4-1BB). In some embodiments, the co-stimulatory domain is derived from 4-1BB and/or CD28 (e.g., a fusion peptide), and comprises an amino acid sequence that is at least 80%, at least 85%, at least 90%, at least 95%, or 100% identical to SEQ ID NO: 152, 157 and 158.

Various CAR molecules and vectors expressing these CAR molecules can be used in the methods described herein. In some embodiments, the CAR molecules specifically bind to CLL1 (e.g., human CLL1). In some embodiments, the CAR comprises the amino acid sequence set forth in any of SEQ ID NOs: 7, 17, 27, 37, 47, 57, 67, 77, 87, 97, 107, 117, 127, and 132-145; or an amino acid sequence that has at least 80%, 85%, 90%, 91%, 92%, 93%, 94%, 95%, 96%, 97%, 98%, or 99% sequence identity thereto.

Exemplary structure of antigen receptors, including the hinge, the transmembrane domain, and the intracellular T cell signaling domain, and methods for engineering and introducing such receptors into cells, are described, for example, in Chandran et al., Immunological Reviews 290.1 (2019): 127-147; Cartellieri, Marc, et al., BioMed Research International 2010 (2010); and PCT Publication No. WO2017173256A1; US2002/131960, US2013/287748, US2013/0149337, U.S. Pat. Nos. 6,451,995, 7,446,190, and 8,252,592; each of which is incorporated herein by reference in its entirety.

The disclosure provides chimeric antigen receptors (CARs) or fragments thereof that specifically bind to CLL1. The CARs or fragments thereof described herein are capable of binding to CLL1.

The disclosure provides CARs or fragments thereof, comprising (a) an extracellular antigen-binding domain that specifically recognizes CLL1; (b) a transmembrane region; and/or (c) an intracellular signaling domain. In some embodiments, the antigen-binding domain of the CARs or fragments thereof described herein are identical to any of the antigen binding fragments described herein (e.g., AS138628, AS138658, AS138943, AS138993, AS141286, AS141297, AS141330, AS141522, AS141553, AS141567, AS141641, AS141701, and AS141819), or a humanized antibody thereof. In some embodiments, the antigen-binding domain of the CARs or fragments thereof described herein comprises one or more (e.g., 1, 2, 3, 4, 5, or 6) scFvs that are connected with the linker peptide described herein.

The CDR sequences of the antigen-binding domain for AS138628 CAR, or related antigen-binding fragment thereof include CDRs of the heavy chain variable domain, SEQ ID NOs: 1, 2, and 3, and CDRs of the light chain variable domain, SEQ ID NOs: 4, 5, and 6.

The CDR sequences of the antigen-binding domain for AS138658 CAR, or related antigen-binding fragment thereof include CDRs of the heavy chain variable domain, SEQ ID NOs: 11, 12, and 13, and CDRs of the light chain variable domain, SEQ ID NOs: 14, 15, and 16.

The CDR sequences of the antigen-binding domain for AS138943 CAR, or related antigen-binding fragment thereof include CDRs of the heavy chain variable domain, SEQ ID NOs: 21, 22, and 23, and CDRs of the light chain variable domain, SEQ ID NOs: 24, 25 and 26.

The CDR sequences of the antigen-binding domain for AS138993 CAR, or related antigen-binding fragment thereof include CDRs of the heavy chain variable domain, SEQ ID NOs: 31, 32, and 33, and CDRs of the light chain variable domain, SEQ ID NOs: 34, 35 and 36.

The CDR sequences of the antigen-binding domain for AS141286 CAR, or related antigen-binding fragment thereof include CDRs of the heavy chain variable domain, SEQ ID NOs: 41, 42, and 43, and CDRs of the light chain variable domain, SEQ ID NOs: 44, 45 and 46.

The CDR sequences of the antigen-binding domain for AS141297 CAR, or related antigen-binding fragment thereof include CDRs of the heavy chain variable domain, SEQ ID NOs: 51, 52, and 53, and CDRs of the light chain variable domain, SEQ ID NOs: 54, 55 and 56.

The CDR sequences of the antigen-binding domain for AS141330 CAR, or related antigen-binding fragment thereof include CDRs of the heavy chain variable domain, SEQ ID NOs: 61, 62, and 63, and CDRs of the light chain variable domain, SEQ ID NOs: 64, 65 and 66.

The CDR sequences of the antigen-binding domain for AS141522 CAR, or related antigen-binding fragment thereof include CDRs of the heavy chain variable domain, SEQ ID NOs: 71, 72, and 73, and CDRs of the light chain variable domain, SEQ ID NOs: 74, 75 and 76.

The CDR sequences of the antigen-binding domain for AS141553 CAR, or related antigen-binding fragment thereof include CDRs of the heavy chain variable domain, SEQ ID NOs: 81, 82, and 83, and CDRs of the light chain variable domain, SEQ ID NOs: 84, 85 and 86.

The CDR sequences of the antigen-binding domain for AS141567 CAR, or related antigen-binding fragment thereof include CDRs of the heavy chain variable domain, SEQ ID NOs: 91, 92, and 93, and CDRs of the light chain variable domain, SEQ ID NOs: 94, 95 and 96.

The CDR sequences of the antigen-binding domain for AS141641 CAR, or related antigen-binding fragment thereof include CDRs of the heavy chain variable domain, SEQ ID NOs: 101, 102, and 103, and CDRs of the light chain variable domain, SEQ ID NOs: 104, 105, and 106.

The CDR sequences of the antigen-binding domain for AS141701 CAR, or related antigen-binding fragment thereof include CDRs of the heavy chain variable domain, SEQ ID NOs: 111, 112, and 113, and CDRs of the light chain variable domain, SEQ ID NOs: 114, 115 and 116.

The CDR sequences of the antigen-binding domain for AS141819 CAR, or related antigen-binding fragment thereof include CDRs of the heavy chain variable domain, SEQ ID NOs: 121, 122, and 123, and CDRs of the light chain variable domain, SEQ ID NOs: 124, 125 and 126.

Also provided herein are engineered receptors (CARs) including (a) a first antigen-binding fragment thereof that is identical to any one of the antigen-binding domain that specifically recognizes CLL1 described herein; and (b) a second antigen-binding fragment thereof that binds to CD33.

The disclosure also provides CARs or fragments thereof, comprising (a) a first extracellular antigen-binding domain that specifically recognizes CLL1; (b) a second extracellular antigen-binding domain that specifically recognizes CD33; (c) a transmembrane region; and/or (d) an intracellular signaling domain. In some embodiments, the heavy and light chains (VH and VL, respectively) for each scFv are placed in sequential order. In some embodiments, the antigen-binding domain that specifically recognizes CD33 is in the N terminal. In some embodiments, the antigen-binding domain that specifically recognizes CLL1 is in the N terminal. In some embodiments, the heavy and light chains of one scFv are inserted between the VH and VL of the other scFv.

In some embodiments, the first antigen-binding domain of the CARs or fragments thereof described herein are identical to any of the antigen binding fragments described herein (e.g., AS138628, AS138658, AS138943, AS138993, AS141286, AS141297, AS141330, AS141522, AS141553, AS141567, AS141641, AS141701, and AS141819), or a humanized antibody thereof. In some embodiments, the antigen-binding domain of the CARs or fragments thereof described herein comprises one or more (e.g., 1, 2, 3, 4, 5, or 6) scFvs that are connected with the linker peptide described herein.

In some embodiments, the second antigen-binding domain of the CARs or fragments thereof described herein are identical to any of the antigen binding fragments of AS141869, AS199772, AS200728C and AS188893, or a humanized antibody thereof. In some embodiments, the antigen-binding domain of the CARs or fragments thereof described herein comprises one or more (e.g., 1, 2, 3, 4, 5, or 6) scFvs that are connected with the linker peptide described herein.

The amino acid sequence for the heavy chain variable region of antibody AS141869 is set forth in SEQ ID NO: 162. The amino acid sequence for the light chain variable region of AS141869 antibody is set forth in SEQ ID NO: 161. The amino acid sequence for the scFv of antibody AS141869 is set forth in SEQ ID NO: 160.

The CDR sequences of the antigen-binding domain for AS141869, or related antigen-binding fragment thereof include CDRs of the heavy chain variable domain, SEQ ID NOs: 175, 176 and 177, and CDRs of the light chain variable domain, SEQ ID NOs: 178, 179 and 180.

The amino acid sequence for the heavy chain variable region of antibody AS199772 is set forth in SEQ ID NO: 166. The amino acid sequence for the light chain variable region of AS199772 antibody is set forth in SEQ ID NO: 165. The amino acid sequence for the scFv of antibody AS199772 is set forth in SEQ ID NO: 164.

The CDR sequences of the antigen-binding domain for AS199772, or related antigen-binding fragment thereof include CDRs of the heavy chain variable domain, SEQ ID NOs: 181, 182 and 183, and CDRs of the light chain variable domain, SEQ ID NOs: 184, 185 and 186.

The amino acid sequence for the heavy chain variable region of antibody AS200728C is set forth in SEQ ID NO: 170. The amino acid sequence for the light chain variable region of AS200728C antibody is set forth in SEQ ID NO: 169. The amino acid sequence for the scFv of antibody AS200728C is set forth in SEQ ID NO: 168.

The CDR sequences of the antigen-binding domain for AS200728C, or related antigen-binding fragment thereof include CDRs of the heavy chain variable domain, SEQ ID NOs: 187, 188 and 189, and CDRs of the light chain variable domain, SEQ ID NOs: 190, 191 and 192.

The amino acid sequence for the heavy chain variable region of antibody AS188893 is set forth in SEQ ID NO: 174. The amino acid sequence for the light chain variable region of AS188893 antibody is set forth in SEQ ID NO: 173. The amino acid sequence for the scFv of antibody AS188893 is set forth in SEQ ID NO: 172.

The CDR sequences of the antigen-binding domain for AS188893, or related antigen-binding fragment thereof include CDRs of the heavy chain variable domain, SEQ ID NOs: 193, 194 and 195, and CDRs of the light chain variable domain, SEQ ID NOs: 196, 197 and 198.

In some embodiments, the first extracellular antigen-binding domain and the second extracellular antigen-binding domain are connected via a linker. Any suitable linkers described herein can be used to link the two extracellular antigen-binding domains. In some embodiments, the linker comprises the amino acid sequence of SGGGGS (SEQ ID NO: 148). In some embodiments, the linker comprises the amino acid sequence of GGGGSGGGGSGGGGSGGGGSGGGGS (SEQ ID NO: 149).

In some embodiments, the VH and VL of one scFv that are inserted between the VH and VL of the other scFv are connected via a linker. Any suitable linkers described herein can be used to link the VH and VL. In some embodiments, the linker comprises the amino acid sequence of GSTSGSGKPGSGEGSTKG (SEQ ID NO: 199). In some embodiments, the VH of one scFv and VL of another scFv are connected via a linker. Any suitable linkers described herein can be used to link the VH of one scFv and VL of another scFv. In some embodiments, the linker comprises the amino acid sequence of GGGGS (SEQ ID NO: 200).

In some embodiments, the VH2 and VL2 of one scFv that are inserted between the VH1 and VL1 of the other scFv are connected via a linker. Any suitable linkers described herein can be used to link the VH2 and VL2. In some embodiments, the linker between VH2 and VL2 comprises the amino acid sequence of GSTSGSGKPGSGEGSTKG (SEQ ID NO: 199). In some embodiments, the VL1 and VH2 are connected via a linker. Any suitable linkers described herein can be used to link the VL1 of and VH2. In some embodiments, the linker between VL1 and VH2 comprises the amino acid sequence of GGGGS (SEQ ID NO: 200). In some embodiments, the VL2 and VH1 are connected via a linker. Any suitable linkers described herein can be used to link the VL2 and VH1. In some embodiments, the linker between VL2 and VH1 comprises the amino acid sequence of GGGGS (SEQ ID NO: 200).

In some embodiments, the second antigen-binding domain of the CARs or fragments thereof described herein are identical to any of the antigen binding fragments described herein (e.g., AS138628, AS138658, AS138943, AS138993, AS141286, AS141297, AS141330, AS141522, AS141553, AS141567, AS141641, AS141701, and AS141819), or a humanized antibody thereof. In some embodiments, the antigen-binding domain of the CARs or fragments thereof described herein comprises one or more (e.g., 1, 2, 3, 4, 5, or 6) scFvs that are connected with the linker peptide described herein.

In some embodiments, the amino acid sequences for scFv of the antigen-binding domain for the CAR, or related antigen binding fragment thereof are humanized (e.g., a sequence can be modified with different amino acid substitutions). In some embodiments, the scFv can have more than one version of humanized sequences.

In some embodiments, the CAR, related antibody or antigen binding fragment thereof described herein can have a heavy chain variable domain (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence.

In some embodiments, the CAR, related antibody or antigen binding fragment thereof described herein can have a light chain variable domain (VL) comprising complementarity determining regions (CDRs) 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence.

The selected VH and VL CDRs 1, 2, 3 amino acid sequences are shown in FIGS. 14A-14B.

In some embodiments, the CAR, related antibody or antigen binding fragment thereof described herein contains a VH containing one, two, or three of the VH CDR1 with zero, one or two amino acid insertions, deletions, or substitutions; VH CDR2 with zero, one or two amino acid insertions, deletions, or substitutions; VH CDR3 with zero, one or two amino acid insertions, deletions, or substitutions.

In some embodiments, the CAR, related antibody or antigen binding fragment thereof described herein contains a VL containing one, two, or three of the VL CDR1 with zero, one or two amino acid insertions, deletions, or substitutions; VL CDR2 with zero, one or two amino acid insertions, deletions, or substitutions; VL CDR3 with zero, one or two amino acid insertions, deletions, or substitutions.

The disclosure also provides CARs or fragments thereof that bind to CLL1. In some embodiments, the CAR, related antibody or antigen binding fragment thereof contains a heavy chain variable region (VH) comprising or consisting of an amino acid sequence that is at least 80%, 85%, 90%, or 95% identical to a selected VH sequence. In some embodiments, the selected VH sequence is selected from SEQ ID NOs: 10, 20, 30, 40, 50, 60, 70, 80, 90, 100, 110, 120, and 130.

In some embodiments, the CAR, related antibody or antigen binding fragment thereof contains a light chain variable region (VL) comprising or consisting of an amino acid sequence that is at least 80%, 85%, 90%, or 95% identical to a selected VL sequence. In some embodiments, the selected VL sequence is selected from SEQ ID NOs: 9, 19, 29, 39, 49, 59, 69, 79, 89, 99, 109, 119, and 129.

The amino acid sequence for AS138628 CAR is set forth in SEQ ID NO: 7. The amino acid sequence for AS138658CAR is set forth in SEQ ID NO: 17. The amino acid sequence for AS138943 CAR is set forth in SEQ ID NO: 27. The amino acid sequence for AS138993 CAR is set forth in SEQ ID NO: 37. The amino acid sequence for AS141286 CAR is set forth in SEQ ID NO: 47. The amino acid sequence for AS141297 CAR is set forth in SEQ ID NO: 57. The amino acid sequence for AS141330 CAR is set forth in SEQ ID NO: 67. The amino acid sequence for AS141522 CAR is set forth in SEQ ID NO: 77. The amino acid sequence for AS141553 CAR is set forth in SEQ ID NO: 87. The amino acid sequence for AS141567 CAR is set forth in SEQ ID NO: 97. The amino acid sequence for AS141641 CAR is set forth in SEQ ID NO: 107. The amino acid sequence for AS141701 CAR is set forth in SEQ ID NO: 117. The amino acid sequence for AS141819 CAR is set forth in SEQ ID NO: 127.

Also provided herein are CARs comprising two scFvs that are connected with the linker peptide described herein (i.e., tandem CARs). For example, the amino acid sequence for Tan1-R-893-943 CAR is set forth in SEQ ID NO: 132. The amino acid sequence for Tan2-S-893-943 CAR is set forth in SEQ ID NO: 133. The amino acid sequence for Tan3-T-893-943 CAR is set forth in SEQ ID NO: 134. The amino acid sequence for Tan4-R-772-943 CAR is set forth in SEQ ID NO: 135. The amino acid sequence for Tan5-S-772-943 CAR is set forth in SEQ ID NO: 136. The amino acid sequence for Tan6-T-772-943 CAR is set forth in SEQ ID NO: 137. The amino acid sequence for Tan7-R-728C-943 CAR is set forth in SEQ ID NO: 138. The amino acid sequence for Tan8-S-728C-943 CAR is set forth in SEQ ID NO: 139. The amino acid sequence for Tan9-T-728C-943 CAR is set forth in SEQ ID NO: 140. The amino acid sequence for Tan10-R-869-567 CAR is set forth in SEQ ID NO: 141.

The disclosure also provides dual receptor systems comprising: (a) a first engineered receptor comprising a first antigen-binding fragment thereof as described herein; and (b) a second engineered receptor comprising a second antigen-binding fragment thereof that binds to CD33.

In some embodiments, the dual receptor system comprises a first CAR comprising (a) a first extracellular antigen-binding domain that specifically recognizes CLL1; a first transmembrane region; and/or a first intracellular signaling domain; and a second CAR comprising (b) a second extracellular antigen-binding domain that specifically recognizes CD33; a second transmembrane region; and/or a second intracellular signaling domain.

Also provided herein are dual receptor systems (dual CAR systems) comprising two CARs described herein, and the two CARs target different molecules (i.e., Dual CARs targeting CD33 and CLL1). For example, the amino acid sequence for AS138943 CAR in Dual1-Para-943-893 CAR and Dual2-Para-943-772 CAR is set forth in SEQ ID NO: 27, the amino acid sequence for AS141567 CAR in Dual3-Para-567-893 CAR and Dual4-Para-567-772 CAR is set forth in SEQ ID NO: 97, the amino acid sequence for AS188893 CAR in Dual1-Para-943-893 and Dual3-Para-567-893 is set forth in SEQ ID NO: 171, the amino acid sequence for AS199772 CAR in Dual4-Para-567-772 is set forth in SEQ ID NO: 163.

Also provided herein are a dual CAR polypeptide comprising two CAR polypeptides that are connected with e.g., a self-cleaving peptide described herein (e.g., P2A), and the two CARs can target different molecules (i.e., Dual CARs targeting CD33 and CLL1). For example, the amino acid sequence for Dual1-Para-943-893 CAR is set forth in SEQ ID NO: 142. The amino acid sequence for Dual2-Para-943-772 CAR is set forth in SEQ ID NO: 143. The amino acid sequence for Dual3-Para-567-893 CAR is set forth in SEQ ID NO: 144. The amino acid sequence for Dual4-Para-567-772 CAR is set forth in SEQ ID NO: 145.

In some embodiments, provided herein are CARs or fragments thereof comprising an amino acid sequence that is at least 90%, 91%, 92%, 93%, 94%, 95%, 96%, 97%, 98%, 99% or 100% identical to any one of SEQ ID NOs: 7, 17, 27, 37, 47, 57, 67, 77, 87, 97, 107, 117, 127, and 132-145. In some embodiments, the CAR described herein comprises an amino acid sequence as set forth in any one of SEQ ID NOs: 7, 17, 27, 37, 47, 57, 67, 77, 87, 97, 107, 117, 127, and 132-145; optionally with about or no more than 0, 1, 2, 3, 4, 5, 6, 7, 8, 9, 10, 15, 20, 25, 30, 35, 40, 45, or 50 amino acid insertions, deletions, or substitutions.

In some embodiments, the chimeric antigen receptors (CARs) or fragments thereof described herein comprises a hinge region. In some embodiments, the hinge region is a membrane-proximal region from CD8, and/or CD28, or an IgG hinge region, or any combination thereof. In some embodiments, the hinge region is a membrane-proximal region of CD8 (e.g., human CD8). In some embodiments, the hinge region is a fusion peptide comprising all or a portion of the membrane-proximal region of CD28 (e.g., human CD28) and all or a portion of the membrane-proximal region of CD8 (e.g., human CD8). In some embodiments, the hinge region comprises the membrane-proximal regions of both CD8 and CD28.

In some embodiments, the chimeric antigen receptors (CARs) or fragments thereof described herein comprises a transmembrane region. In some embodiments, the transmembrane domain is a transmembrane domain of 4-1BB/CD137, an alpha chain of a T cell receptor, a beta chain of a T cell receptor, CD3 epsilon, CD4, CD5, CD8, CD8 alpha, CD9, CD16, CD19, CD22, CD28, CD33, CD37, CD45, CD64, CD80, CD86, CD134, CD137, CD154, or a zeta chain of a T cell receptor, or any combination thereof. In some embodiments, the transmembrane region is a transmembrane region from CD8 (e.g., human CD8). In some embodiments, the hinge region and the transmembrane region are directly joined. In some embodiments, the joined hinge region and the transmembrane region comprises an amino acid sequence that is at least 80%, at least 85%, at least 90%, at least 95%, or 100% identical to SEQ ID NO: 151. In some embodiments, the transmembrane region is a fusion peptide comprising all or a portion of the transmembrane region of CD28 (e.g., human CD28) and all or a portion of the transmembrane region of CD8 (e.g., human CD8). In some embodiments, the transmembrane region comprises the transmembrane regions of both CD8 and CD28.

In some embodiments, the chimeric antigen receptors (CARs) or fragments thereof described herein comprises an intracellular signaling domain. In some embodiments, the intracellular signaling domain comprises an activating cytoplasmic signaling domain, which is capable of inducing a primary activation signal in an immune cell (e.g., a T cell). In some embodiments, the activating cytoplasmic signaling domain is a T cell receptor (TCR) component. In some embodiments, the activating cytoplasmic signaling domain comprises an immunoreceptor tyrosine-based activation motif (ITAM). In some embodiments, the intracellular signaling domain comprises an amino acid sequence derived from CD3 zeta, FcR gamma, FcR beta, CD3 gamma, CD3 delta, CD3 epsilon, CD5, CD22, CD79a, CD79b, CD278 (ICOS), FceRI, CD66d, DAP10, DAP12, or combinations thereof. In some embodiments, the intracellular signaling domain comprises a functional signaling domain of CD3 zeta (e.g., a human CD3 zeta). In some embodiments, the intracellular signaling domain comprises an amino acid sequence that is at least 80%, at least 85%, at least 90%, at least 95%, or 100% identical to SEQ ID NO: 150.

In some embodiments, the chimeric antigen receptors (CARs) or fragments thereof described herein comprises a costimulatory signaling domain. In some embodiments, the costimulatory signaling domain is between the transmembrane domain and the intracellular signaling domain. In some embodiments, the costimulatory signaling domain comprises a functional signaling domain from a protein selected from the group consisting of a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1, CD11a/CD18, 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, CD19a, and a CD83 ligand. In some embodiments, the costimulatory signaling domain comprises a functional signaling domain from OX40, CD28, 4-1BB, ICOS, or a signaling portion thereof. In some embodiments, the costimulatory signaling domain comprises an intracellular signaling domain of 4-1BB (e.g., human 4-1BB). In some embodiments, the costimulatory signaling domain comprises an amino acid sequence that is at least 80%, at least 85%, at least 90%, at least 95%, or 100% identical to SEQ ID NO: 152. In some embodiments, the costimulatory signaling domain comprises an intracellular signaling domain of CD28 (e.g., human CD28). In some embodiments, the costimulatory signaling domain comprises intracellular signaling domains of both CD28 (e.g., human CD28) and 4-1BB (e.g., human 4-1BB). In some embodiments, the costimulatory signaling domain is a fusion peptide comprising all or a portion of the intracellular signaling domain of CD28 (e.g., human CD28) and all or a portion of the intracellular signaling domain of 4-1BB (e.g., human 4-1BB).

In some embodiments, the hinge region, transmembrane region, and/or intracellular signaling domain (e.g., costimulatory signaling domain and/or activating cytoplasmic signaling domain) of CARs or fragments thereof described herein are derived from a first generation, a second generation, a third generation, or a fourth generation CAR structure. Details of the structural features of CARs can be found, e.g., in Jackson, Hollie J., et al., Nature Reviews Clinical Oncology 13.6 (2016): 370; and Subklewe, Marion, et al., Transfusion Medicine and Hemotherapy 46.1 (2019): 15-24; each of which is incorporated herein by reference.

In some embodiments, the CAR is dual chain CAR, ligand-based CAR, T cell receptor fusion constructs (TRuCs), universal immune receptors (UIR), or tandem CARs (tanCARs). In some embodiments, the CAR is used in connection with bispecific T cell engagers (BiTEs). These CAR constructs are described e.g., Hughes-Parry et al., International journal of molecular sciences 21.1 (2020): 204, which is incorporated herein by reference in its entirety.

### CAR, Antibody, Antigen-Binding Fragment Characteristics

In some embodiments, the CAR, antibodies, or antigen-binding fragments thereof as described herein can increase immune response, activity or number of immune cells (e.g., T cells, CD8+ T cells, CD4+ T cells, macrophages, antigen presenting cells) by at least 10%, 20%, 30%, 40%, 50%, 60%, 70%, 80%, 90%, 100%, 2 folds, 3 folds, 5 folds, 10 folds, or 20 folds, as compared to that of immune cells that do not express the CAR, antibodies, or antigen-binding fragments thereof.

In some implementations, the antibody (or antigen-binding fragments thereof) specifically binds to CLL1 with a dissociation rate (koff or Kd) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹ or greater than 0.000001 s⁻¹.

In some embodiments, kinetic association rates (kon or Ka) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

Binding affinities can be deduced from the quotient of the kinetic rate constants (KD=koff/kon). In some embodiments, KD (Kd) for the antibody, antigen-binding fragments thereof, or molecules derived therefrom (e.g., CAR), is less than 1×10⁻⁶ M, less than 1×10⁻⁷ M, less than 1×10⁻⁸ M, less than 1×10⁻⁹ M, or less than 1×10⁻¹⁰ M. In some embodiments, the KD is less than 100 nM, 50 nM, 30 nM, 20 nM, 15 nM, 10 nM, 9 nM, 8 nM, 7 nM, 6 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In some embodiments, KD is greater than 1×10⁻⁷ M, greater than 1×10⁻⁸ M, greater than 1×10⁻⁹ M, greater than 1×10⁻¹⁰ M, greater than 1×10⁻¹¹ M, or greater than 1×10⁻¹² M. General techniques for measuring the affinity of an antibody for an antigen include, e.g., enzyme-linked immunosorbent assay (ELISA), Radioimmunoassay (RIA), fluorescence-activated cell sorting (FACS), and surface plasmon resonance (SPR). In some embodiments, the antibody binds to human CLL1. In some embodiments, the antibody binds to the extracellular domain (ECD) of human CLL1. In some embodiments, the antibody binds to monkey CLL1 (e.g., cynomolgus). In some embodiments, the antibody binds to a cell expressing CLL1.

### Engineered Cells

The present disclosure provides engineered cells (e.g., immune cells, T cells, NK cells, tumor-infiltrating lymphocytes) that express CAR, and/or various proteins as described herein. These engineered cells can be used to treat various disorders or disease as described herein (e.g., CLL1-associated cancer).

In various embodiments, the cell that is engineered can be obtained from e.g., humans and non-human animals. In various embodiments, the cell that is engineered can be obtained from bacteria, fungi, humans, rats, mice, rabbits, monkeys, pig or any other species. Preferably, the cell is from humans, rats or mice. In some embodiments, the cells are mouse lymphocytes and engineered (e.g., transduced) to express the CAR, or antigen-binding fragment thereof. In some embodiments, the cell is obtained from humans. In various embodiments, the cell that is engineered is a blood cell. Preferably, the cell is a leukocyte (e.g., a T cell), lymphocyte or any other suitable blood cell type. In some embodiments, the cell is a peripheral blood cell. In some embodiments, the cell is a tumor-infiltrating lymphocyte (TIL). In some embodiments, the cell is a T cell, B cell or NK cell. In some embodiments, the cells are human peripheral blood mononuclear cells (PBMCs). In some embodiments, the human PBMCs are CD3+ cells. In some embodiments, the human PBMCs are CD8+ cells or CD4+ cells.

In some embodiments, the cell is a T cell. In some embodiments, the T cells can express a cell surface receptor that recognizes a specific antigenic moiety on the surface of a target cell. The cell surface receptor can be a wild type or recombinant T cell receptor (TCR), a chimeric antigen receptor (CAR), or any other surface receptor capable of recognizing an antigenic moiety that is associated with the target cell. T cells can be obtained by various methods known in the art, e.g., in vitro culture of T cells (e.g., tumor infiltrating lymphocytes) isolated from patients. Genetically modified T cells can be obtained by transducing T cells (e.g., isolated from the peripheral blood of patients), with a viral vector. In some embodiments, the T cells are CD4+ T cells, CD8+ T cells, or regulatory T cells. In some embodiments, the T cells are T helper type 1 T cells and T helper type 2 T cells. In some embodiments, the T cell is an αβT cell. In alternate embodiments, the T cell is a γδT cell. In some embodiments, the T cells are central memory T cells. In some embodiments, the T cells are effector memory T cells. In some embodiments, the T cells are naïve T cells.

In some embodiments, the cell is an NK cell. In some embodiments, preparation of the engineered cells includes one or more culture and/or preparation steps. The cells for introduction of the binding molecule, e.g., CAR, can be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

In some embodiments, the cells are stem cells, such as multipotent and pluripotent stem cells, including induced pluripotent stem cells (iPSCs). The cells can be primary cells, such as those isolated directly from a subject and/or isolated from a subject and frozen. In some embodiments, the stem cells are cultured with additional differentiation factors to obtain desired cell types (e.g., T cells).

Different cell types can be obtained from appropriate isolation methods. The isolation methods include the separation of different cell types based on the expression or presence in the cell of one or more specific molecules, such as surface markers, e.g., surface proteins, intracellular markers, or nucleic acid. In some embodiments, any known method for separation based on such markers can be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

Also provided are methods, nucleic acids, compositions, and kits, for expressing the binding molecules, and for producing the genetically engineered cells expressing such binding molecules. The genetic engineering generally involves introduction of a nucleic acid encoding the therapeutic molecule, e.g., CAR, polypeptides, fusion proteins, into the cell, such as by retroviral transduction, transfection, or transformation. In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical application.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors. In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), or spleen focus forming virus (SFFV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In some embodiments, the vector is a lentivirus vector. In some embodiments, recombinant nucleic acids are transferred into T cells via electroporation. In some embodiments, recombinant nucleic acids are transferred into T cells via transposition. Other methods of introducing and expressing genetic material in immune cells include calcium phosphate transfection, protoplast fusion, cationic liposome-mediated transfection, tungsten particle-facilitated microparticle bombardment and strontium phosphate DNA co-precipitation. Many of these methods are descried e.g., in WO2019195486, which is incorporated herein by reference in its entirety. In some embodiments, the T cells are pre-activated, e.g., using anti-CD3/CD28 particles, for about 12 hours, about 24 hours, about 36 hours, about 48 hours, or about 60 hours prior to transduction. In some embodiments, the transduced T cells are harvested on day 5, day 6, day 7, day 8, day 9, day 10, day 11, or day 12 post transduction.

In some embodiments, the transfection efficiency of the virus-infected T cells described herein is at least 10%, at least 15%, at least 20%, at least 25%, at least 30%, at least 35%, 40%, at least 45%, at least 50%, at least 55%, at least 60%, at least 65%, at least 70%, at least 75%, or at least 80%. In some embodiments, the viability of the transduced T cells is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% on day 0, day 1, day 2, day 3, day 4, or day 5 post transduction. In some embodiments, the viability of the transduced T cells is at least or about 80%, at least or about 90%, at least or about 100%, at least or about 110%, at least or about 120% as compared to the viability of untransduced T cells, on day 0, day 1, day 2, day 3, day 4, or day 5 (e.g., on day 5) post transduction.

In some embodiments, the T cell expansion fold is at least 1 fold, 2 folds, 3 folds, 4 folds, 5 folds, 10 folds, 15 folds, 20 folds, 25 folds, 30 folds, 35 folds, 40 folds, 45 folds, or 50 folds, on day 0, day 1, day 2, day 3, day 4, or day 5 post transduction. In some embodiments, the T cell expansion fold of the transduced T cells is at least or about 50%, at least or about 60%, at least or about 70%, at least or about 80%, at least or about 90% as compared to that of untransduced T cells, on day 0, day 1, day 2, day 3, day 4, or day 5 (e.g., on day 5) post transduction.

Also provided are populations of engineered cells, compositions containing such cells and/or enriched for such cells, such as in which cells expressing the CAR make up at least 15%, 20%, 25%, 30%, 35%, 40%, 50%, 60%, 70%, 80%, 90%, 91%, 92%, 93%, 94%, 95%, 96%, 97%, 98%, 99%, or more percent of the total cells in the composition or cells of a certain type such as T cells, CD8+ or CD4+ cells.

In some embodiments, the engineered cells (e.g., CAR-T cells) are co-cultured with target cells for at least or about 1 hour, 2 hours, 3 hours, 4 hours, 5 hours, 6 hours, 12 hours, 16 hours, 18 hours, 1 day, 2 days, 3 days, or longer, such that the engineered cells (e.g., CAR-T cells) can be activated.

In some embodiments, the in vitro cytotoxicity of the engineered cells described herein (e.g., CAR-T cells) is determined. In some embodiments, the engineered cells are incubated with the target cells at an E:T ratio of about 1:0.1, about 1:0.2, about 1:0.3, about 1:0.5, about 1:1, about 1:2, about 1:5, about 1:10, about 1:20, about 1:50. In some embodiments, the incubation is about 8 hours, about 10 hours, about 12 hours, about 14 hours, about 16 hours, about 20 hours, about 22 hours, about 24 hours, about 36 hours, or about 48 hours.

In some embodiments, the cytotoxicity is calculated by the equation below ([LDH]_{E+T}: the LDH released from E/T co-incubation, [LDH]E: the LDH released from Effector only, [LDH]ₘₐₓ: the LDH released from target cells treated with Triton X-100, [LDH]ₘᵢₙ: the LDH released from untreated target cells):

$$Cytotoxicity \% = \frac{{[ LDH ]}_{E + T} - {[ LDH ]}_{E} - {[ LDH ]}_{min}}{{[ LDH ]}_{max} - {[ LDH ]}_{min}} \times 100$$

In some embodiment, the in vitro cytotoxicity of the engineered cells described herein (e.g., CAR-T cells) is at least 15%, at least 20%, at least 30%, at least 40%, at least 50%, at least 60%, at least 70%, at least 80%, at least 90%, or at least 95%.

In some embodiments, the long-term cytotoxicity of the engineered cells (e.g., CAR-T cells) is determined, e.g., by re-challenging the engineered cells. Exemplary re-challenging procedures of CAR-T cells can be found, e.g., in Wang, Dongrui, et al., Journal of Visualized Experiments: JoVE 144 (2019); Wang D, et al., JCI Insight 2018, 3(10); Lange et al., Cancer Discov. 2021 Feb. 9, candisc.0896.2020; each of which is incorporated herein by reference in its entirety.

In some embodiments, the engineered cells are re-challenged for at least 1, 2, 3, 4, 5, or 6 times. In some embodiments, the calculated cytotoxicity (Cytotoxicity %) is determined after each re-challenge. In some embodiments, after the first re-challenged, the calculated cytotoxicity of the engineered cells described herein is at least 70%, at least 75%, at least 80%, at least 85%, at least 90%, or at least 95%. In some embodiments, after the second re-challenge, the calculated cytotoxicity of the engineered cells described herein is at least 80%, at least 90%, or at least 95%. In some embodiments, after the third re-challenge, the calculated cytotoxicity of the engineered cells described herein is at least 30%, at least 40%, at least 50%, at least 60%, at least 70%, at least 75%, at least 80%, at least 85%, at least 90%, or at least 95%. In some embodiments, after the fourth re-challenge, the calculated cytotoxicity of the engineered cells described herein is at least 15%, at least 20%, at least 30%, at least 40%, at least 50%, at least 60%, at least 70%, at least 80%, at least 90%, or at least 95%. In some embodiments, after the fifth re-challenge, the calculated cytotoxicity of the engineered cells described herein is at least 15%, at least 20%, at least 30%, at least 40%, at least 50%, at least 60%, at least 70%, at least 80%, at least 90%, or at least 95%. In some embodiments, after the sixth re-challenge, the calculated cytotoxicity of the engineered cells described herein is at least 0%, at least 5%, at least 10%, at least 15%, at least 20%, at least 30%, at least 40%, at least 50%, at least 60%, at least 70%, at least 80%, at least 90%, or at least 95%. In some embodiments, the maximum re-challenge number (i.e., the number of re-challenge times before tumor cells outgrow) of the engineered cells described herein is at least 5 times, 6 times, 7 times, 8 times, 9 times, or 10 times.

In some embodiments, the calculated cytotoxicity of the engineered cells having a tandem CAR described herein is at least 1 fold, at least 2 folds, at least 3 folds, at least 4 folds, or at least 5 folds as compared to that of the engineered cells having a CAR with a single extracellular scFv, after 1 re-challenge, 2 re-challenges, 3 re-challenges, 4 re-challenges, 5 re-challenges, or 6 re-challenges.

In some embodiments, when the engineered cells (e.g., CAR-T cells) are co-cultured with target cells, population of the engineered cells increases by at least or about 1 fold, 2 folds, 3 folds, 4 folds, 5 folds, 10 folds, 20 folds, 30 folds, 40 folds, 50 folds, 60 folds, 70 folds, 80 folds, 90 folds, 100 folds, 150 folds, 200 folds, or more, after 1 re-challenge, 2 re-challenges, 3 re-challenges, 4 re-challenges, 5 re-challenges, or 6 re-challenges, as compared to the initial population of the engineered cells.

In some embodiments, concentration of the cytokines (e.g., IFN-γ, GM-CSF, and/or TNF-α) released by the engineered cells (e.g., CAR-T cells) described herein is determined by homogeneous time resolved fluorescence (HTRF) assays.

In some embodiments, the engineered cells (e.g., CAR-T cells) described herein increase cytokine (e.g., IFN-γ, GM-CSF, and/or TNF-α) expression or secretion by at least or about 1 fold, 2 folds, 3 folds, 4 folds, 5 folds, 10 folds, 20 folds, 30 folds, 40 folds, 50 folds, 60 folds, 70 folds, 80 folds, 90 folds, 100 folds, 500 folds, 1000 folds, 2000 folds, 3000 folds, 4000 folds, 5000 folds, 10000 folds, or more when co-cultured with the target cells, as compared to the cytokine expression or secretion level of the untransduced cells (e.g., T cells).

In some embodiments, the engineered cells (e.g., CAR-T cells) described herein release at least 5000 pg/mL INF-δ, at least 6000 pg/mL INF-δ, at least 7000 pg/mL INF-δ, at least 8000 pg/mL INF-δ, at least 9000 pg/mL INF-δ, at least 10000 pg/mL INF-δ, at least 11000 pg/mL INF-δ, at least 12000 pg/mL INF-δ, at least 13000 pg/mL INF-δ, at least 14000 pg/mL INF-δ, or at least 15000 pg/mL INF-δ, when co-cultured with the target cells.

In some embodiments, the engineered cells (e.g., CAR-T cells) described herein release at least 1000 pg/mL GM-CSF, at least 1200 pg/mL GM-CSF, at least 1400 pg/mL GM-CSF, at least 1600 pg/mL GM-CSF, at least 1800 pg/mL GM-CSF, at least 2000 pg/mL GM-CSF, at least 2200 pg/mL GM-CSF, at least 2400 pg/mL GM-CSF, at least 2600 pg/mL GM-CSF, at least 2800 pg/mL GM-CSF, at least 3000 pg/mL GM-CSF, at least 3200 pg/mL GM-CSF, at least 3400 pg/mL GM-CSF, when co-cultured with the target cells.

In some embodiments, the engineered cells (e.g., CAR-T cells) described herein release at least 100 pg/mL INF-δ, at least 200 pg/mL INF-δ, at least 300 pg/mL INF-δ, at least 400 pg/mL INF-δ, at least 500 pg/mL INF-δ, at least 600 pg/mL INF-δ, at least 700 pg/mL INF-δ, at least 800 pg/mL INF-δ, at least 900 pg/mL INF-δ, at least 1000 pg/mL INF-δ, at least 1200 pg/mL INF-δ, at least 1400 pg/mL INF-δ, at least 1600 pg/mL INF-δ, at least 1800 pg/mL INF-7, at least 2000 pg/mL INF-δ, at least 2200 pg/mL INF-δ, at least 2400 pg/mL INF-δ, at least 2600 pg/mL INF-δ, at least 2800 pg/mL INF-δ, at least 3000 pg/mL INF-δ, at least 3500 pg/mL INF-δ, at least 4000 pg/mL INF-δ, at least 4500 pg/mL INF-δ, at least 5000 pg/mL INF-δ, at least 5500 pg/mL INF-δ, or at least 6000 pg/mL INF-δ, when co-cultured with the target cells.

In some embodiments, the engineered cells (e.g., CAR-T cells) described herein release at least 200 pg/mL GM-CSF, 400 pg/mL GM-CSF, 600 pg/mL GM-CSF, 800 pg/mL GM-CSF, 1000 pg/mL GM-CSF, 1200 pg/mL GM-CSF, 1400 pg/mL GM-CSF, 1600 pg/mL GM-CSF, 1800 pg/mL GM-CSF, 2000 pg/mL GM-CSF, 2200 pg/mL GM-CSF, 2400 pg/mL GM-CSF, 2600 pg/mL GM-CSF, 2800 pg/mL GM-CSF, 3000 pg/mL GM-CSF, 3200 pg/mL GM-CSF, 3400 pg/mL GM-CSF, 3600 pg/mL GM-CSF, 3800 pg/mL GM-CSF, 4000 pg/mL GM-CSF, 4500 pg/mL GM-CSF, 5000 pg/mL GM-CSF, 5500 pg/mL GM-CSF, 6000 pg/mL GM-CSF, 6500 pg/mL GM-CSF, 7000 pg/mL GM-CSF, 7500 pg/mL GM-CSF, or 8000 pg/mL GM-CSF, when co-cultured with the target cells.

In some embodiments, cytokine (e.g., IFN-γ, GM-CSF, and/or TNF-α) expression of the engineered cells having a tandem or dual-targeting CAR described herein (e.g., any one of Tan1-R-893-943, Tan2-S-893-943, Tan3-T-893-943, Tan4-R-772-943, Tan5-S-772-943, Tan6-T-772-943, Tan7-R-728C-943, Tan8-S-728C-943, Tan9-T-728C-943, Tan10-R-869-567, Dual1-Para-943-893, Dual2-Para-943-772, Dual3-567-893, Dual4-Para-567-772 CARs) is at least or about 100%, at least or about 110%, at least or about 120%, at least or about 130%, at least or about 140%, at least or about 150%, or more, as compared to that of the engineered cell having a CAR with a single extracellular scFv, after 1 re-challenge, 2 re-challenges, 3 re-challenges, 4 re-challenges, 5 re-challenges, or 6 re-challenges.

In some embodiments, the cells are human PBMCs and engineered (e.g., transduced) to express the CAR, or antigen-binding fragment thereof.

In some embodiments, the in vitro cytotoxicity of the engineered cells described herein (e.g., CAR-T cells) is determined. In some embodiments, the in vitro cytotoxicity of the engineered cells described herein (e.g., CAR-T cells) is determined using a U937-Luc mouse xenograft model. In some embodiments, mice treated with the engineered cells described herein (e.g., CAR-T cells) are tumor-free after 2 weeks, after 3 weeks, after 4 weeks, or after 5 weeks post-treatment.

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant polypeptides or fragments thereof by recombinant techniques.

A vector is a construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

The present disclosure provides a recombinant vector comprising a nucleic acid construct suitable for genetically modifying a cell, which can be used for treatment of pathological disease or condition.

Any vector or vector type can be used to deliver genetic material to the cell. These vectors include but are not limited to plasmid vectors, viral vectors, bacterial artificial chromosomes (BACs), yeast artificial chromosomes (YACs), and human artificial chromosomes (HACs). Viral vectors can include but are not limited to recombinant retroviral vectors, recombinant lentiviral vectors, recombinant adenoviral vectors, foamy virus vectors, recombinant adeno-associated viral (AAV) vectors, hybrid vectors, and plasmid transposons (e.g., sleeping beauty transposon system, and PiggyBac transposon system) or integrase based vector systems. Other vectors that are known in the art can also be used in connection with the methods described herein.

In some embodiments, the vector is a viral vector. The viral vector can be grown in a culture medium specific for viral vector manufacturing. Any suitable growth media and/or supplements for growing viral vectors can be used in accordance with the embodiments described herein. In some embodiments, the viral vector contains constitutive promoters to facilitate expression, exemplary constitutive promoters contemplated herein include, but are not limited to, Cytomegalovirus (CMV) promoters, human elongation factors-1alpha (hEF1α), ubiquitin C promoter (UbiC), phosphoglycerokinase promoter (PGK), simian virus 40 early promoter (SV40), and chicken 3-Actin promoter coupled with CMV early enhancer (CAGG). In some embodiments, the constitutive promoter is a hEF1α promoter.

In some embodiments, the vector used is a recombinant retroviral vector. A retroviral vector is capable of directing the expression of a nucleic acid molecule of interest. A retrovirus is present in the RNA form in its viral capsule and forms a double-stranded DNA intermediate when it replicates in the host cell. Similarly, retroviral vectors are present in both RNA and double-stranded DNA forms. The retroviral vector also includes the DNA form which contains a recombinant DNA fragment and the RNA form containing a recombinant RNA fragment. The vectors can include at least one transcriptional promoter/enhancer, or other elements which control gene expression. Such vectors can also include a packaging signal, long terminal repeats (LTRs) or portion thereof, and positive and negative strand primer binding sites appropriate to the retrovirus used. Long terminal repeats (LTRs) are identical sequences of DNA that repeat many times (e.g., hundreds or thousands of times) found at either end of retrotransposons or proviral DNA formed by reverse transcription of retroviral RNA. They are used by viruses to insert their genetic material into the host genomes. Optionally, the vectors can also include a signal which directs polyadenylation, selectable markers such as Ampicillin resistance, Neomycin resistance, TK, hygromycin resistance, phleomycin resistance histidinol resistance, or DHFR, as well as one or more restriction sites and a translation termination sequence. For example, such vectors can include a 5′ LTR, a leading sequence, a tRNA binding site, a packaging signal, an origin of second strand DNA synthesis, and a 3′ LTR or a portion thereof. Additionally, retroviral vector used herein can also refers to the recombinant vectors created by removal of the retroviral gag, pol, and env genes and replaced with the gene of interest.

In some embodiments, the vector or construct can contain a single promoter that drives the expression of one or more nucleic acid molecules. In some embodiments, such promoters can be multicistronic (bicistronic or tricistronic). For example, in some embodiments, transcription units can be engineered as a bicistronic unit containing an IRES (internal ribosome entry site), which allows coexpression of gene products (e.g., encoding CAR and an antibody or antigen binding fragment thereof) by a message from a single promoter. Alternatively, in some cases, a single promoter may direct expression of an RNA that contains, in a single open reading frame (ORF), two or three genes (e.g., encoding CAR and/or an antibody or antigen binding fragment thereof) separated from one another by sequences encoding a self-cleavage peptide (e.g., P2A or T2A) or a protease recognition site (e.g., furin). The ORF thus encodes a single polyprotein, which, either during (in the case of 2A e.g., T2A) or after translation, is cleaved into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream.

Various cell lines can be used in connection with the vectors as described herein. Exemplary eukaryotic cells that may be used to express polypeptides include, but are not limited to, COS cells, including COS 7 cells; HEK293 cells, including HEK293-6E cells; CHO cells, including CHO-S, DG44. Lec13 CHO cells, and FUT8 CHO cells; PER.C6® cells; and NSO cells. In some embodiments, a particular eukaryotic host cell is selected based on its ability to make desired post-translational modifications to the antibodies or CAR molecule. For example, in some embodiments, CHO cells produce polypeptides that have a higher level of sialylation than the same polypeptide produced in HEK293 cells. In one aspect, the disclosure relates to a cell comprising the vector or the pair of vectors as described herein.

The present disclosure also provides a nucleic acid sequence comprising a nucleotide sequence encoding any of the antibodies, CAR, antigen binding fragments thereof, and/or CAR-derived binding molecules (including e.g., functional portions and functional variants thereof, polypeptides, or proteins described herein). “Nucleic acid” as used herein can include “polynucleotide,” “oligonucleotide,” and “nucleic acid molecule,” and generally means a polymer of DNA or RNA, which can be single-stranded or double-stranded, synthesized or obtained from natural sources, which can contain natural, non-natural or altered nucleotides. Furthermore, the nucleic acid comprises complementary DNA (cDNA). It is generally preferred that the nucleic acid does not comprise any insertions, deletions, inversions, and/or substitutions. However, it can be suitable in some instances, as discussed herein, for the nucleic acid to comprise one or more insertions, deletions, inversions, and/or substitutions.

The nucleic acids as described herein can be constructed based on chemical synthesis and/or enzymatic ligation reactions using procedures known in the art. For example, a nucleic acid can be chemically synthesized using naturally occurring nucleotides or variously modified nucleotides. In some of any such embodiments, the nucleotide sequence is codon-optimized.

The present disclosure also provides the nucleic acids comprising a nucleotide sequence complementary to the nucleotide sequence of any of the nucleic acids described herein or a nucleotide sequence which hybridizes under stringent conditions to the nucleotide sequence of any of the nucleic acids described herein.

In some embodiments, the nucleotide sequence encoding the CARs are separated by a peptide sequence that causes ribosome skipping. In some embodiments, the peptide that causes ribosome skipping is a P2A or T2A peptide. In some embodiments, the nucleic acid is synthetic. In some embodiments, the nucleic acid is cDNA.

In certain embodiments, the polypeptide comprises a signal peptide. In some embodiments, the signal peptide comprises a sequence that is at least 80%, at least 85%, at least 90%, at least 95%, or 100% identical to SEQ ID NO: 146.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3% 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

In some embodiments, the nucleic acid sequence is at least or about 10, 20, 30, 40, 50, 60, 70, 80, 90, 100, 110, 120, 130, 150, 200, 250, 300, 350, 400, 500, or 600 nucleotides. In some embodiments, the amino acid sequence is at least or about 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, or 900 amino acid residues. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, or 900 amino acid residues.

### Method for Preparation of Engineered Cells

The present disclosure provides a method or process for preparing, manufacturing and/or using the engineered cells for treatment of pathological diseases or conditions.

The cells for introduction of the protein described herein, e.g., CAR, can be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g., transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, or non-human primate. In some embodiments, the cells are isolated from mouse lymph nodes.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge. In some aspects, a washing step is accomplished by tangential flow filtration (TFF). In some embodiments, the cells are resuspended in a variety of biocompatible buffers after washing, such as, for example, Ca²⁺/Mg²⁺ free PBS. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient.

In some embodiments, the method comprises one or more steps of: e.g., isolating the T cells from a patient's blood; transducing the population T cells with a viral vector including the nucleic acid construct encoding a genetically engineered antigen receptor; expanding the transduced cells in vitro; and/or infusing the expanded cells into the patient, where the engineered T cells will seek and destroy antigen positive tumor cells. In some embodiments, the method further comprises: transfection of T cells with the viral vector containing the nucleic acid construct.

In some embodiments, the methods involve introducing any vectors described herein into a cell in vitro or ex vivo. In some embodiments, the vector is a viral vector and the introducing is carried out by transduction. In some embodiments, the cell is transduced for at least or about 1 hour, 2 hours, 3 hours, 4 hours, 5 hours, 6 hours, 12 hours, 18 hours, 1 day, 2 days, 3 days, 4 days, 5 days, 6 days, 1 week, 2 weeks, 3 weeks, or longer. In some embodiments, the methods further involve introducing into the cell one or more agent, wherein each of the one or more agent is independently capable of inducing a genetic disruption of a T cell receptor alpha constant (TRAC) gene and/or a T cell receptor beta constant (TRBC) gene. In some embodiments, the one or more agent is an inhibitory nucleic acid (e.g., siRNA). In some embodiments, the one or more agent is a fusion protein comprising a DNA-targeting protein and a nuclease or an RNA-guided nuclease (e.g., a clustered regularly interspaced short palindromic nucleic acid (CRISPR)-associated nuclease).

The transfection of T cells can be achieved by using any standard method such as calcium phosphate, electroporation, liposomal mediated transfer, microinjection, biolistic particle delivery system, or any other known methods by skilled artisan. In some embodiments, transfection of T cells is performed using the calcium phosphate method.

The present disclosure provides a method to create a personalized anti-tumor immunotherapy. Genetically engineered T cells can be produced from a patient's blood cells. These engineered T cells are then reinfused into the patient as a cellular therapy product.

### Methods of Treatment

The antibodies and antigen-binding fragments thereof, CARs, and immune cells disclosed herein can be used for various therapeutic purposes. In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of antibodies or antigen binding fragments thereof, or engineered cells expressing CAR, to a subject in need thereof (e.g., a subject having, or identified or diagnosed as having, a cancer).

In some embodiments, the subject has CLL1-positive cancer. In some embodiments, the subject has acute myeloid leukemia (AML). In some embodiments, the subject has liver cancer (e.g., hepatocellular carcinoma), glioma, lung cancer, colorectal cancer, head and neck cancer, stomach cancer, renal cancer, urothelial cancer, testis cancer, breast cancer, cervical cancer, endometrial cancer, and/or ovarian cancer. In some embodiments, the subject has squamous cell lung carcinoma, or solid tumor. In some embodiments, the subject has a CNS tumor, thyroid cancer, gastrointestinal cancer, skin cancer, sarcoma, urogenital cancer, and/or germ cell tumor.

In some embodiments, the compositions and methods disclosed herein can be used for treatment of patients at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the therapeutic agent and/or therapeutic compositions is to be administered, a severity of symptoms and a route of administration, and thus administration can be determined on an individual basis.

As used herein, the term “delaying development of a disease” refers to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, can be delayed.

An effective amount can be administered in one or more administrations. By way of example, an effective amount of a composition is an amount sufficient to ameliorate, stop, stabilize, reverse, inhibit, slow and/or delay progression of a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective may vary, depending on, inter alia, patient history as well as other factors such as the type (and/or dosage) of compositions used.

Effective amounts and schedules for administrations may be determined empirically, and making such determinations is within the skill in the art. Those skilled in the art will understand that the dosage that must be administered will vary depending on, for example, the mammal that will receive the treatment, the route of administration, the particular type of therapeutic agents and other drugs being administered to the mammal. Guidance in selecting appropriate doses can be found in the literature. In addition, a treatment does not necessarily result in the 100% or complete treatment or prevention of a disease or a condition. There are multiple treatment/prevention methods available with a varying degree of therapeutic effect which one of ordinary skill in the art recognizes as a potentially advantageous therapeutic mean.

In some aspects, the present disclosure also provides methods of diagnosing a disease/condition in a mammal, wherein the CARs, antibodies, or antigen binding fragments, interact with the sample(s) obtained from a subject to form a complex, wherein the sample can comprise one more cells, polypeptides, proteins, nucleic acids, antibodies, or antigen binding portions, blood, whole cells, lysates thereof, or a fraction of the whole cell lysates, e.g., a nuclear or cytoplasmic fraction, a whole protein fraction, or a nucleic acid fraction thereof, wherein the detection of the complex is the indicative of presence of a condition in the mammal, wherein the condition is cancer or infection. Further, the detection of the complex can be in any number of way known in the art but not limited to, ELISA, Flow cytometery, Fluorescence in situ hybridization (FISH), Polymerase chain reaction (PCR), microarray, southern blotting, electrophoresis, Phage analysis, chromatography and more. Thus, the treatment methods can further include determining whether a subject can benefit from a treatment as disclosed herein, e.g., by determining whether the subject has infection or cancer.

In any of the methods described herein, the engineered cells, optionally with at least one additional therapeutic agent, can be administered to the subject at least once a week (e.g., once a week, twice a week, three times a week, four times a week, once a day, twice a day, or three times a day). In some embodiments, at least two different engineered cells (e.g., cells expressing different CARs) are administered in the same composition (e.g., a liquid composition). In some embodiments, engineered cells and at least one additional therapeutic agent are administered in the same composition (e.g., a liquid composition). In some embodiments, engineered cells and at least one additional therapeutic agent are administered in two different compositions. In some embodiments, the at least one additional therapeutic agent is administered as a pill, tablet, or capsule. In some embodiments, the at least one additional therapeutic agent is administered in a sustained-release oral formulation. In some embodiments, the one or more additional therapeutic agents can be administered to the subject prior to, concurrently with, or after administering the engineered cells to the subject.

In some embodiments, the additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2, 3-dioxygenase-1) (IDO1) (e.g., epacadostat). In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject. In some embodiments, the additional therapeutic agent is selected from asparaginase, busulfan, carboplatin, cisplatin, daunorubicin, doxorubicin, fluorouracil, gemcitabine, hydroxyurea, methotrexate, paclitaxel, rituximab, vinblastine, vincristine and/or combinations thereof.

In some embodiments, the additional therapeutic agent is an anti-OX40 antibody, an anti-PD-1 antibody, an anti-PD-L1 antibody, an anti-PD-L2 antibody, an anti-LAG-3 antibody, an anti-TIGIT antibody, an anti-BTLA antibody, an anti-CTLA-4 antibody, or an anti-GITR antibody.

### Compositions and Formulations

The present disclosure provides compositions (including pharmaceutical and therapeutic compositions) containing the engineered cells and populations thereof, produced by the methods disclosed herein. Also provided are methods, e.g., therapeutic methods for administrating the engineered cells and compositions thereof to subjects, e.g., patients or animal models (e.g., mice).

Compositions including the engineered cells for administration, including pharmaceutical compositions and formulations, such as unit dose form compositions including the number of cells for administration in a given dose or fraction thereof are provided. The pharmaceutical compositions and formulations can include one or more optional pharmaceutically acceptable carrier or excipient. In some embodiments, the composition includes at least one additional therapeutic agent.

A pharmaceutically acceptable carrier refers to an ingredient in a pharmaceutical composition, other than an active ingredient. The pharmaceutically acceptable carrier does not interfere with the active ingredient and is nontoxic to a subject. A pharmaceutically acceptable carrier can include, but is not limited to, a buffer, excipient, stabilizer, or preservative. The pharmaceutical formulation refers to process in which different substances and/or agents are combined to produce a final medicinal product. The formulation studies involve developing a preparation of drug acceptable for patient. Additionally, a preparation which is in such form as to permit the biological activity of an active ingredient contained therein to be effective, and which contains no additional components which are unacceptably toxic to a subject to which the formulation would be administered.

In some embodiments, the choice of carrier is determined in part by the particular cell (e.g., T cell or NK cell) and/or by the method of administration. A variety of suitable formulations are available. For example, the pharmaceutical composition can contain preservatives. Suitable preservatives can include, for example, methylparaben, propylparaben, sodium benzoate, and benzalkonium chloride. In some embodiments, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

Suitable buffering agents include, for example, citric acid, sodium citrate, phosphoric acid, potassium phosphate, and various other acids and salts. In some embodiments, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition can also contain more than one active ingredient useful for a particular indication, disease, or condition being treated with the engineered cells, preferably those with activities complementary to the cells, where the respective activities do not adversely affect one another. Such active ingredients are suitably present in combination in amounts that are effective for the purpose intended. Thus, in some embodiments, the pharmaceutical composition can further include other pharmaceutically active agents or drugs, such as checkpoint inhibitors, fusion proteins, chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/orvincristine.

The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

The cells and compositions can be administered using standard administration techniques, formulations, and/or devices. Administration of the cells can be autologous or heterologous. For example, immunoresponsive T cells or progenitors can be obtained from one subject, and administered to the same subject or a different, compatible subject after genetically modifying them in accordance with various embodiments described herein. Peripheral blood derived immunoresponsive T cells or their progeny (e.g., in vivo, ex vivo or in vitro derived) can be administered via localized injection, including catheter administration, systemic injection, localized injection, intravenous injection, or parenteral administration. Usually, when administering a therapeutic composition (e.g., a pharmaceutical composition containing a genetically modified immunoresponsive cell), it is generally formulated in a unit dosage injectable form (solution, suspension, emulsion).

Formulations disclosed herein include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts can in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility can be readily accomplished, e.g., by filtration through sterile filtration membranes.

The compositions or pharmaceutical compositions as described herein can be included in a container, pack, or dispenser together with instructions for administration.

### Methods of Administration

Provided are also methods of administering the cells, populations, and compositions, and uses of such cells, populations, and compositions to treat or prevent diseases, conditions, and disorders, including cancers. In some embodiments, the methods described herein can reduce the risk of the developing diseases, conditions, and disorders as described herein.

In some embodiments, the cells, populations, and compositions, described herein are administered to a subject or patient having a particular disease or condition to be treated, e.g., via adoptive cell therapy, such as adoptive T cell therapy. In some embodiments, cells and compositions prepared by the provided methods, such as engineered compositions and end-of-production compositions following incubation and/or other processing steps, are administered to a subject, such as a subject having or at risk for the disease or condition. In some aspects, the methods thereby treat, e.g., ameliorate one or more symptom of, the disease or condition, such as by lessening tumor burden in cancer expressing an antigen recognized by the engineered T cells.

Methods for administration of cells for adoptive cell therapy are known and can be used in connection with the provided methods and compositions. For example, adoptive T cell therapy methods are described, e.g., in U.S. 2003/0170238; U.S. Pat. No. 4,690,915; Rosenberg, Nature reviews Clinical oncology 8.10 (2011): 577; Themeli et al., Nature biotechnology 31.10 (2013): 928; Tsukahara et al., Biochemical and biophysical research communications 438.1 (2013): 84-89; Davila et al., PloS one 8.4 (2013); each of which is incorporated herein by reference in its entirety.

In some embodiments, the cell therapy, e.g., adoptive T cell therapy, is carried out by autologous transfer, in which the T cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the T cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject has been treated with a therapeutic agent targeting the disease or condition, e.g., the tumor, prior to administration of the cells or composition containing the cells. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at high risk of relapse, and thus the cells are administered prophylactically, e.g., to reduce the likelihood of or prevent relapse. In some embodiments, the subject has not received prior treatment with another therapeutic agent.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8+ and CD4+ T cells, are administered at or within a tolerated difference of a desired dose of total cells, such as a desired dose of T cells. In some embodiments, the desired dose is a desired number of cells or a desired number of cells per unit of body weight of the subject to whom the cells are administered, e.g., cells/kg. In some embodiments, the desired dose is at or above a minimum number of cells or minimum number of cells per unit of body weight. In some embodiments, among the total cells, administered at the desired dose, the individual populations or sub-types are present at or near a desired output ratio (such as CD4+ to CD8+ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some embodiments, the desired dose is a desired number of cells of the sub-type or population, or a desired number of such cells per unit of body weight of the subject to whom the cells are administered, e.g., cells/kg. In some embodiments, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4+ to CD8+ cells, and/or is based on a desired fixed or minimum dose of CD4+ and/or CD8+ cells.

In certain embodiments, the cells or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 10⁴ and at or about 10⁹ cells/kilograms (kg) body weight, such as between 10⁵ and 10⁶ cells/kg body weight, for example, at least or at least about or at or about 1×10⁵ cells/kg, 1.5×10⁵ cells/kg, 2×10⁵ cells/kg, or 1×10⁶ cells/kg body weight. For example, in some embodiments, the cells are administered at, or within a certain range of error of, between at or about 10⁴ and at or about 10⁹ T cells/kilograms (kg) body weight, such as between 10⁵ and 10⁶ T cells/kg body weight, for example, at least or at least about or at or about 1×10⁵ T cells/kg, 1.5×10⁵ T cells/kg, 2×10⁵ T cells/kg, or 1×10⁶ T cells/kg body weight.

In some embodiments, the cells are administered at or within a certain range of error of between at or about 10⁴ and at or about 10⁹ CD4+ and/or CD8+ cells/kilograms (kg) body weight, such as between 10⁵ and 10⁶ CD4+ and/or CD8+ cells/kg body weight, for example, at least or at least about or at or about 1×10⁵ CD4+ and/or CD8+ cells/kg, 1.5×10⁵ CD4+ and/or CD8+ cells/kg, 2×10⁵ CD4+ and/or CD8+ cells/kg, or 1×10⁶ CD4+ and/or CD8+ cells/kg body weight.

In some embodiments, the cells are administered at or within a certain range of error of, greater than, and/or at least about 1×10⁶, about 2.5×10⁶, about 5×10⁶, about 7.5×10⁶, or about 9×10⁶ CD4+ cells, and/or at least about 1×10⁶, about 2.5×10⁶, about 5×10⁶, about 7.5×10⁶, or about 9×10⁶ CD8+ cells, and/or at least about 1×10⁶, about 2.5×10⁶, about 5×10⁶, about 7.5×10⁶, or about 9×10⁶ T cells. In some embodiments, the cells are administered at or within a certain range of error of between about 10⁸ and 10¹² or between about 10¹⁰ and 10¹¹ T cells, between about 10⁸ and 10¹² or between about 10¹⁰ and 10¹¹ CD4+ cells, and/or between about 10⁸ and 10¹² or between about 10¹⁰ and 10¹¹ CD8+ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios. for example, in some embodiments, the desired ratio (e.g., ratio of CD4+ to CD8+ cells) is between at or about 1:5 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges. In some aspects, the CAR described here provides improved expression and activity, thereby providing therapeutic effects even at a low effector to target (E:T) ratio.

Optimal response to therapy can depend on the ability of the engineered recombinant receptors such as CARs, to be consistently and reliably expressed on the surface of the cells and/or bind the target antigen. For example, in some cases, properties of certain recombinant receptors, e.g., CARs, can affect the expression and/or activity of the recombinant receptor, in some cases when expressed in a cell, such as a human T cell, used in cell therapy. In some contexts, the level of expression of particular recombinant receptors, e.g., CARs, can be low, and activity of the engineered cells, such as human T cells, expressing such recombinant receptors, may be limited due to poor expression or poor signaling activity. In some cases, consistency and/or efficiency of expression of the recombinant receptor, and activity of the receptor is limited in certain cells or certain cell populations of available therapeutic approaches. In some cases, a large number of engineered T cells (a high effector to target (E:T) ratio) is required to exhibit functional activity. In some embodiments, the desired ratio (E:T ratio) is between at or about 1:10 and at or about 10:1 (or greater than about 1:10 and less than about 10:1), or between at or about 1:1 and at or about 10:1 (or greater than about 1:1 and less than about 5:1), such as between at or about 2:1 and at or about 10:1. In some embodiments, the E:T ratio is greater than or about 1:1, 2:1, 3:1, 4:1, 5:1, 6:1, 7:1, 8:1, 9:1, or 10:1. In some embodiments, the E:T ratio is about 3:1, about 1:1, or about 0.3:1.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

The cells described herein can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

Following administration of the cells, the biological activity of the engineered cell populations in some embodiments is measured, e.g., by any of a number of known methods. Parameters to assess include specific binding of engineered T cells to the antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., Journal of immunotherapy (Hagerstown, Md.: 1997) 32.7 (2009): 689 and Hermans et al., Journal of immunological methods 285.1 (2004): 25-40. In certain embodiments, the biological activity of the cells is measured by assaying expression and/or secretion of one or more cytokines, such as CD107a, IFN-γ, IL-2, and TNF. In some aspects, the biological activity is measured by assessing clinical outcome, such as reduction in tumor burden or load.

Repeated dosing methods are provided in which a first dose of cells is given followed by one or more second consecutive doses. The timing and size of the multiple doses of cells generally are designed to increase the efficacy and/or activity and/or function of engineered cells as described herein, when administered to a subject in adoptive therapy methods. The methods involve administering a first dose, generally followed by one or more consecutive doses, with particular time frames between the different doses.

In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose, provided in multiple individual compositions or infusions, over a specified period of time (e.g., no more than 3 days). Thus, in some contexts, the first or consecutive dose is a single or continuous administration of the specified number of cells, given or initiated at a single point in time. In some contexts, however, the first or consecutive dose is administered in multiple injections or infusions over a limited time period (e.g., no more than three days), such as once a day for three days or for two days or by multiple infusions over a single day period.

The cells of the first dose are administered in a single pharmaceutical composition. In some embodiments, the cells of the consecutive dose are administered in a single pharmaceutical composition.

In some embodiments, the cells of the first dose are administered in a plurality of compositions, collectively containing the cells of the first dose. In some embodiments, the cells of the consecutive dose are administered in a plurality of compositions, collectively containing the cells of the consecutive dose. In some aspects, additional consecutive doses can be administered in a plurality of compositions over a period of no more than 3 days.

With reference to a prior dose, such as a first dose, the term “consecutive dose” refers to a dose that is administered to the same subject after the prior, e.g., first, dose without any intervening doses having been administered to the subject in the interim. Nonetheless, the term does not encompass the second, third, and/or so forth, injection or infusion in a series of infusions or injections comprised within a single split dose. Thus, unless otherwise specified, a second infusion within a one, two or three-day period is not considered to be a “consecutive” dose as used herein. Likewise, a second, third, and so-forth in the series of multiple doses within a split dose also is not considered to be an “intervening” dose in the context of the meaning of “consecutive” dose. Thus, unless otherwise specified, a dose administered a certain period of time, greater than three days, after the initiation of a first or prior dose, is considered to be a “consecutive” dose even if the subject receives a second or subsequent injection or infusion of the cells following the initiation of the first dose, so long as the second or subsequent injection or infusion occurred within the three-day period following the initiation of the first or prior dose.

Thus, unless otherwise specified, multiple administrations of the same cells over a period of up to 3 days is considered to be a single dose, and administration of cells within 3 days of an initial administration is not considered a consecutive dose and is not considered to be an intervening dose for purposes of determining whether a second dose is “consecutive” to the first.

In some embodiments, multiple consecutive doses are given, in some aspects using the same timing guidelines as those with respect to the timing between the first dose and first consecutive dose, e.g., by administering a first and multiple consecutive doses.

In some embodiments, the timing between the first dose and first consecutive dose, or a first and multiple consecutive doses, is such that each consecutive dose is given within a period of time is greater than about 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days or more. In some embodiments, the consecutive dose is given within a time period that is less than about 28 days after the administration of the first or immediately prior dose. The additional multiple additional consecutive dose or doses also are referred to as subsequent dose or subsequent consecutive dose.

The size of the first and/or one or more consecutive doses of cells are generally designed to provide improved efficacy and/or reduced risk of toxicity. In some aspects, a dosage amount or size of a first dose or any consecutive dose is any dosage or amount as described above. In some embodiments, the number of cells in the first dose or in any consecutive dose is between about 0.5×10⁶ cells/kg body weight of the subject and 5×10⁶ cells/kg, between about 0.75×10⁶ cells/kg and 3×10⁶ cells/kg or between about 1×10⁶ cells/kg and 2×10⁶ cells/kg.

As used herein, “first dose” is used to describe the timing of a given dose being prior to the administration of a consecutive or subsequent dose. The term does not necessarily imply that the subject has never before received a dose of cell therapy or even that the subject has not before received a dose of the same cells or cells expressing the same recombinant receptor or targeting the same antigen.

In some embodiments, multiple doses can be administered to a subject over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of cancer).

### EXAMPLES

The disclosure is further described in the following examples, which do not limit the scope of the disclosure described in the claims.

### Example 1: Generation of Anti-CLL1 IgG1

### Preparation of Recombinant Extracellular Domains of CLL1 Proteins

The extracellular protein domains (ECD) of CLL1 proteins from human or cynomolgus monkey were prepared by transient expression in human embryonic kidney HEK293 cells transfected with plasmids encoding the respective amino acid sequences.

Each expression plasmid was complexed with 293FECTIN™ (Life Technologies) and added to suspension-cultured 293-F cells (derived from HEK293 cells). Eight days post-transfection, the culture supernatants were collected and the corresponding soluble protein was purified by IMAC (GE Healthcare) to produce a protein batch.

### Immunization

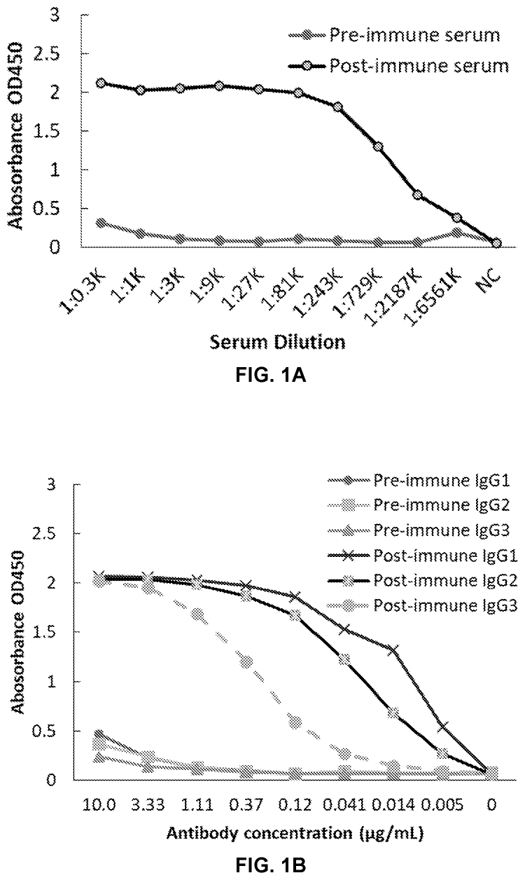

Camels were immunized with each recombinant ECD of CLL1 under all current animal welfare regulations. For immunization, the antigen was formulated as an emulsion with CFA (Complete Freund's adjuvant; primary immunization) or IFA (incomplete Freund's adjuvant; boost immunizations). The antigen was administered subcutaneously at the neck. Each animal received 6 injections of the emulsion, containing 200 μg of CLL1-His protein in CFA emulsion and 5 subsequent injections of CLL1-His protein in IFA emulsion at two-week intervals. At different time points during immunization, 10 ml blood samples were collected from the animal and sera were prepared. The induction of an antigen specific humoral immune response was verified using the serum samples using an ELISA-based assay with immobilized CLL1-His protein. As shown in FIGS. 1A-1B, the post-immune serum and immunoglobulins (including both 4-chain antibodies and heavy-chain only antibodies) specifically bind to the CLL1-His protein in a concentration-dependent manner. Four days after the last immunization, a blood sample of 180 ml was collected. Peripheral blood lymphocytes (PBLs), as the genetic source of the conventional and camel heavy chain immunoglobulins, were isolated from the blood sample using a Ficoll-Paque gradient (Amersham Biosciences), yielding 2.5×10⁸ PBLs.

### Library Construction

RNA extracted from PBLs was used as starting material for RT-PCR to amplify IgG1 encoding gene fragments. These fragments were cloned into an in-house phagemid vector. The vector encoded a C-terminal His-Tag that was in-frame with each IgG1 coding sequence. The library size was about 3×10⁹. Phage libraries were prepared according to standard protocols and stored after filter-sterilization at 4° C. for further use.

### Selections and High-Throughput Screening

The phage libraries were screened by solid panning as well as cell-based panning. Only a single round of selection was performed for each of the two panning conditions. Each selection output was analyzed to determine an enrichment factor (i.e., number of phages present in the eluate relative to control), diversity and percentage of CLL1 positive clones based on ELISA results. Based on these parameters, the best clones were chosen for further analysis. To this end, the output from each selection was re-cloned as a pool into a soluble expression vector for high-throughput screening. The expression vector encodes a C-terminal His-tag that is in-frame with each IgG1 coding sequence. Colonies were picked and grown in 96-deep-well plates (1 mL volume/well) and induced by adding IPTG and 0.1% Triton for IgG1 expression in the supernatant.

The supernatants were first screened for their ability to bind to the corresponding CLL1 protein using an ELISA assay. The positive binders were sequenced and unique clones were selected for further characterization.

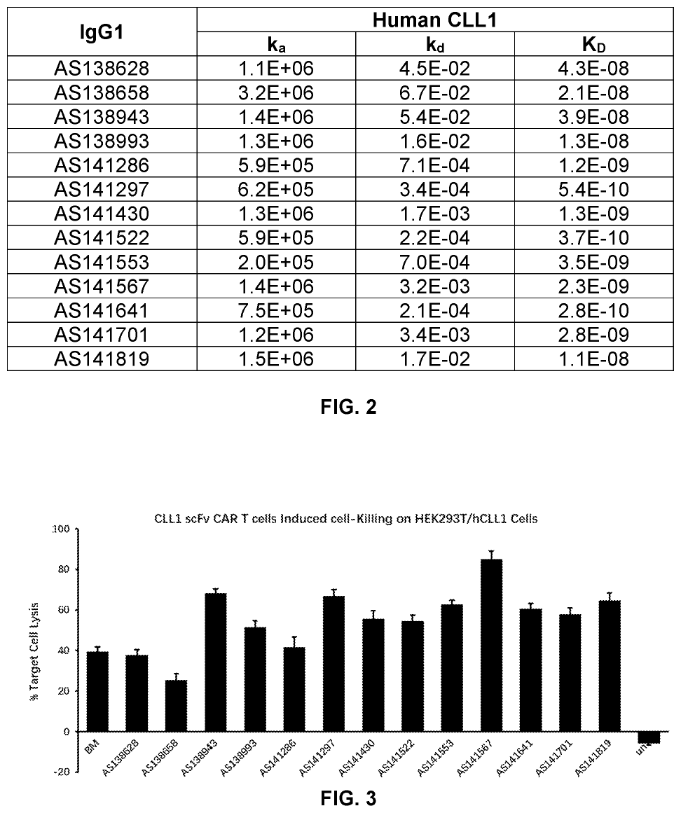

The unique clones were grown in 2YT medium and induced by IPTG for IgG1 expression in the supernatant. The supernatants of unique binders were analyzed for their ability to bind to a CLL1-expressing HEK293/huCLL1 cell line and cancer cell line AMLL193 using a FACS assay. Affinities of selected binders in supernatants to recombinant human and cynomolgus monkey CLL1 proteins were determined by surface plasmon resonance (SPR) on a BIACORE® T200 instrument. The dissociation phase was used to calculate the kd values for each IgG1. Binding data of selected anti-CLL1 IgG1 to recombinant human CLL1 protein are shown in FIG. 2.

### Example 2. Generation and Evaluation of Anti-CLL1 Single CAR Constructs

### Preparation of Lentivirus

The lentivirus packaging plasmid mixture including pCMV-AR-8.47 and pMD2.G (Addgene, Cat #12259) was pre-mixed with each vector PLLV-hEFla-CLL1 comprising a CAR construct at a pre-optimized ratio with polyethylenimine. The mixture was then added to the HEK293 cells. Supernatants from the cells were collected after overnight incubation. The virus-containing supernatants were filtered through a 0.45 μm PES filter, followed by ultra-centrifugation to pellet the lentivirus. The virus pellets were rinsed with pre-chilled PBS. The virus was aliquoted and stored at −80° C. immediately. The virus titer was determined by measurement of transduction efficiency to supT1 cell line using a flow cytometry assay.

### Collection and Transduction of T Lymphocytes

Leukocytes were collected from healthy donors by apheresis. Peripheral blood mononuclear cells (PBMCs) were isolated using FICOLL-PAQUE™ PLUS Media (GE Healthcare, Cat #17-5442-02) according to manufacturer's protocol. Human T cells were purified from PMBCs using a Pan-T cell isolation kit (Miltenyi, Cat #130-096-535), following manufacturer's protocol. The purified T cells were subsequently pre-activated for 48 hours with a human T cell activation/expansion kit (Miltenyi, Cat #130-091-441) according to manufacturer's protocol in which anti-CD3/CD28 MACSiBead particles were added at a bead-to-cell ratio of 1:2. The pre-activated T cells were transduced with each lentivirus stock in the presence of 7 μg/ml polybrene. The transduced cells were then transferred to the cell culture incubator for transgene expression under suitable conditions.

### In Vitro Cytotoxicity Assay

For quick evaluation of anti-tumor activities of CAR-T cells in vitro, LDH (lactate dehydrogenase) assay for cytotoxicity was performed. On day 6 or day 11 after transduction, transduced T cells were harvested and co-incubated with target cells (CLL1-HEK293T) at an E/T ratio (Effector:CAR-T/Target) of 10:1 for 12 hours. Un-transduced T cells (“UnT”) from the same batch were used as a negative control. The assay was performed following the manufacturer's manual (Roche, 11644793001). The cytotoxicity was calculated by the equation below ([LDH]_{E+T}: the LDH released from E/T co-incubation, [LDH]E: the LDH released from Effector only, [LDH]ₘₐₓ: the LDH released from target cells treated with Triton X-100, [LDH]ₘᵢₙ: the LDH released from untreated target cells):

$$Cytotoxicity \% = \frac{{[ LDH ]}_{E + T} - {[ LDH ]}_{E} - {[ LDH ]}_{min}}{{[ LDH ]}_{max} - {[ LDH ]}_{min}} \times 100$$

A sdAb based CAR T was used as benchmark (“BM CAR”, SEQ ID NO: 131). The BM CAR was described in WO2020/052543 A1, which is incorporated herein by reference in its entirety. As shown in FIG. 3, most anti-CLL1 scFv based CAR-T cells showed stronger cytotoxicity against CLL1-HEK293T cells than BM CAR-T cells.

### IFN-γ and GM-CSF Secretion Detected by HTRF

Another measure of effector T-cell activation and proliferation is the production of effector cytokines such as IFN-γ and GM-CSF. Supernatant from the in vitro cytotoxicity assay were collected to assess CAR-induced cytokine release. HTRF assays for IFN-γ (Cisbio, Cat #62HIFNGPEH) and GM-CSF were performed according to the manufacturer's manual.

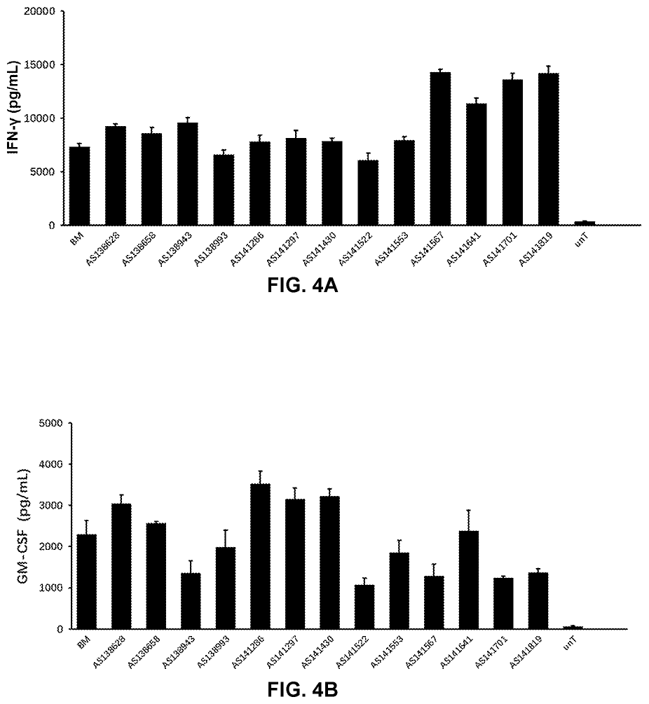

The corresponding cytokine release results are shown in FIGS. 4A-4B. All anti-CLL1 CAR-T cells exhibited potent killing activity against CLL1-HEK293T cells, and released IFN-γ and GM-CSF in response to CLL1-HEK293T cells.

### Long-Term Co-Culture Assay

To evaluate the long-term killing efficacy of CAR-T cells, long-term co-culture assays were performed, which mimic the dynamic killing process in vivo. Tumor cell lines (e.g., THP-1) were labeled with CFSE (SIGMA-ALDRICH, Cat #21888-25MG-F). Transduced or non-transduced T cells (2×10⁵/well) were co-cultured with tumor cell lines (e.g., CFSE-THP-1 cells, 2×10⁵ or 4×10⁵/well) at an E:T ratio of 1:1 or 1:2 in 24-well plates, in the absence of exogenous cytokines (IL-2). Part of the cells were harvested and stained for CD3 after 2 or 3 days' co-culture. Tumor cells were identified by CFSE signal. For serial co-culture assays, the remaining T cells were then re-challenged with fresh CFSE-THP-1 cells at the same E:T ratio. Co-cultures were carried on until tumor cells outgrew. The T cell proliferation rate at each time point is calculated by dividing the number of T cells at the time point by the number of T cells at a previous time point.

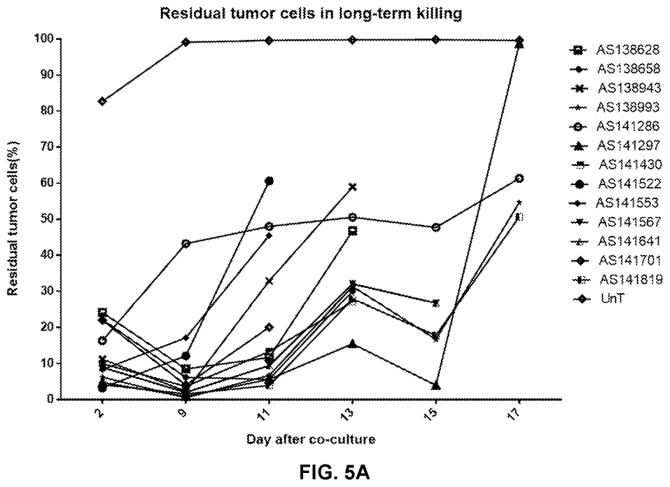

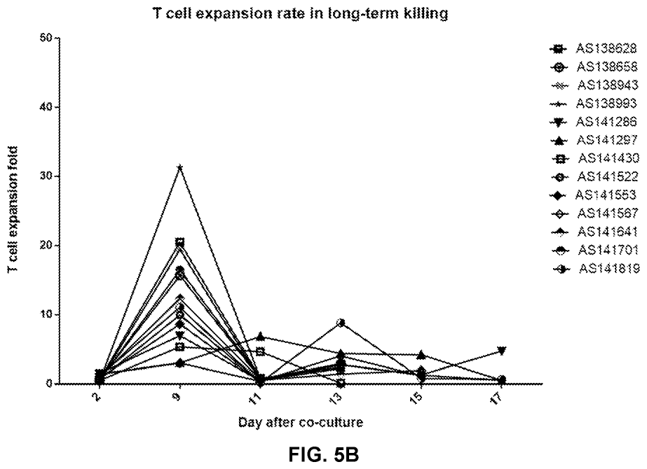

Representative result of long-term co-culture assay by FACS detection was shown in FIG. 5A. Calculated T cell proliferation rate from the same experiment were shown in FIG. 5B, respectively. The data indicate that the persistence and proliferation of anti-CLL1 scFv based CAR-T performed well.

### In Vivo Efficacy Evaluation in Mouse Xenograft Model

U937-Luc were cultured, re-suspended in HBSS−/− and injected intravenously at 2×10⁶ cells per mouse. Bioluminescent imaging (BLI) was conducted weekly or biweekly post tumor inoculation to monitor model development. The animals were randomized based on the BLI photon numbers and animal body weight. After randomization, a single dose of CAR-T cells or UnT cells were infused intravenously. Weekly BLI imaging was performed to record tumor growth.

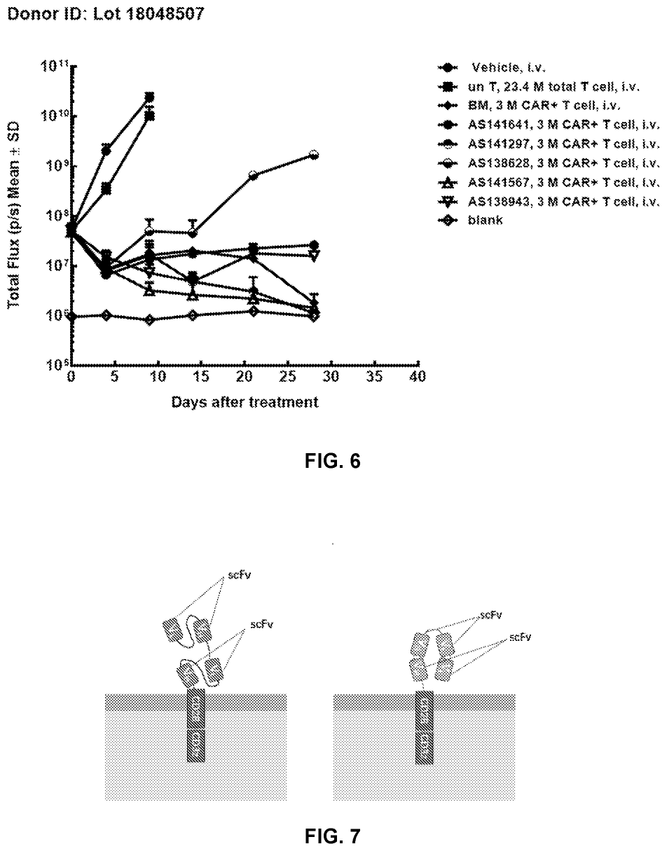

As shown in FIG. 6, mice treated with AS141297 or AS141567 CAR-T cells were tumor free (BLI around 10⁶) after 4 weeks post infusion, while mice treated with UnT or vehicle exhibited rapid tumor progression and had to be euthanized before the end of the experiment.

### Example 3. Generation and Evaluation of Anti-CD33/CLL1 Tandem CAR Constructs

Exemplary tandem CARs as shown in FIG. 7 were constructed by fusing two binding domains specifically recognizing different targets (CD33 and CLL1) via a peptide linker to form the extracellular domain in a single CAR molecule. Anti-CD33/CLL1 tandem CARs were cloned into a lentiviral expression vector with the intracellular co-stimulatory sequence of CD28 and intracellular domain of CD3 zeta. The CAR constructs were cloned into an expression vector with an EF1α promoter for expression.

### Generation of Anti-CD33/CLL1 Tandem CAR-T Cells

Lentiviruses encoding the tandem CARs (Tan 1-Tan 10) were prepared as described in Example 2. T lymphocytes were collected and transduced with the lentiviruses according to the protocol in Example 2.

### Evaluation of In Vitro Activity of Anti-CD33/CLL1 Tandem CAR-T Cells

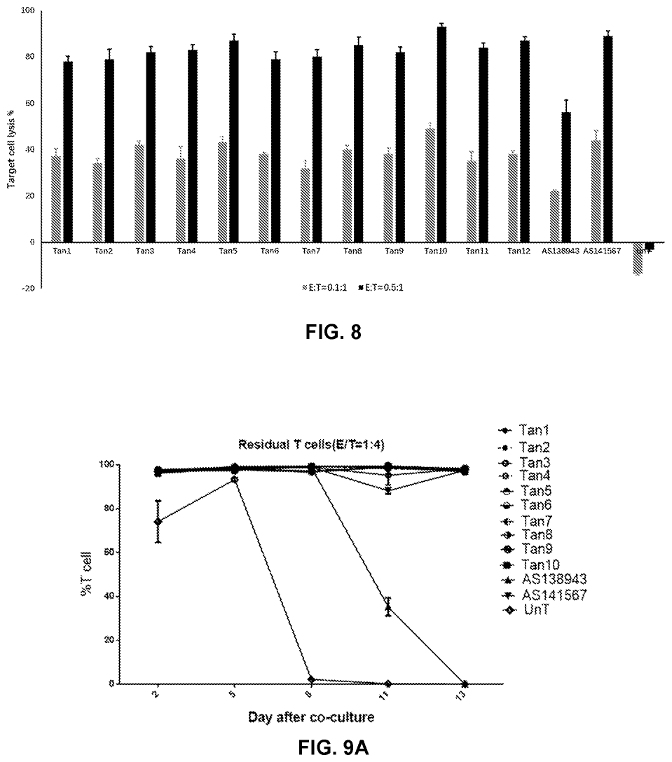

The anti-tumor activities of tandem CAR-T cells were assessed using the in vitro LDH assay as described in Example 2. As shown in FIG. 8, in vitro cytotoxicity of tandem CARs against THP-1 was comparable with any anti-CLL1 single CAR-T cells, which indicated that tandem CARs against two targets would be more efficient for tumor elimination.

### Long-Term Co-Culture Assay

To evaluate the long-term killing efficacy of CAR-T cells, long-term co-culture assays were performed, which mimic the dynamic killing process in vivo. AML tumor cell lines (e.g., U937) were labeled with CFSE (SIGMA-ALDRICH, Cat #21888-25MG-F). Transduced or non-transduced T cells (1×10⁵/well) were co-cultured with tumor cells (e.g., CFSE-U937 cells, 4×10⁵ well) at an E:T ratio of 1:4 in 24-well plates, in the absence of exogenous cytokines (IL-2). Part of the cells were harvested and stained for CD3 after 2 or 3 days' co-culture. Tumor cells were identified by CFSE+ signal. For serial co-culture assays, the remaining T cells were then re-challenged with fresh CFSE-U937 cells at the same E:T ratio. Co-cultures were carried on until tumor cells outgrew. The T cell proliferation rate at each time point was calculated by dividing the number of T cells at the time point by the initial number of T cells.

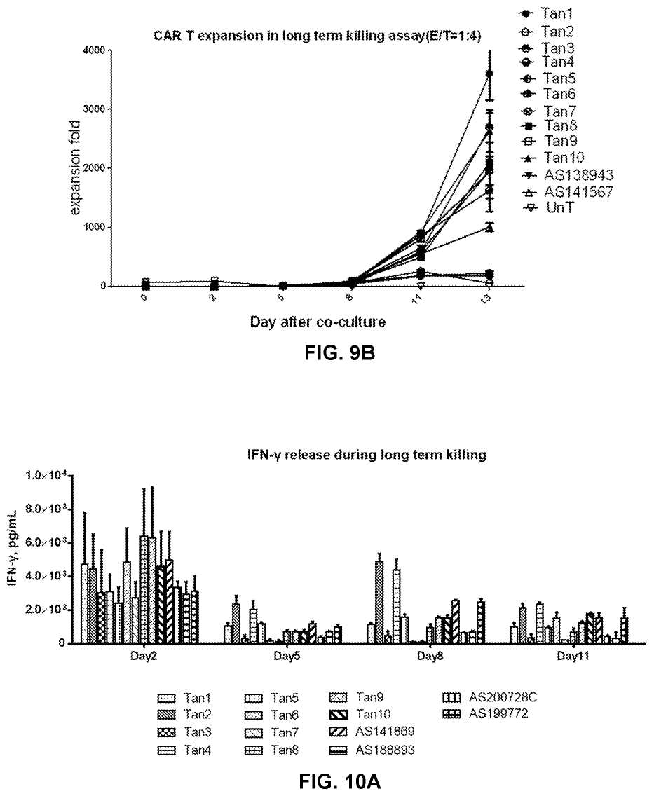

The killing efficacy of various tandem CAR-T cells in the repeated tumor stimulation assay is shown in FIG. 9A. The single-target anti-CLL1 CAR-T cells were exhausted after 3 rounds of tumor stimulation, while most tandem CAR-T cells persisted until 4 or 5 rounds of tumor stimulation. In addition, tandem CAR-T cells proliferated faster than AS138943, AS141567 CAR-T cells in vitro (FIG. 9B). These results demonstrate that CD33/CLL1 tandem CAR-T cells have more potent anti-tumor activity than single-target CAR-T cells in vitro.

### IFN-γ and GM-CSF Secretion Detected by HTRF

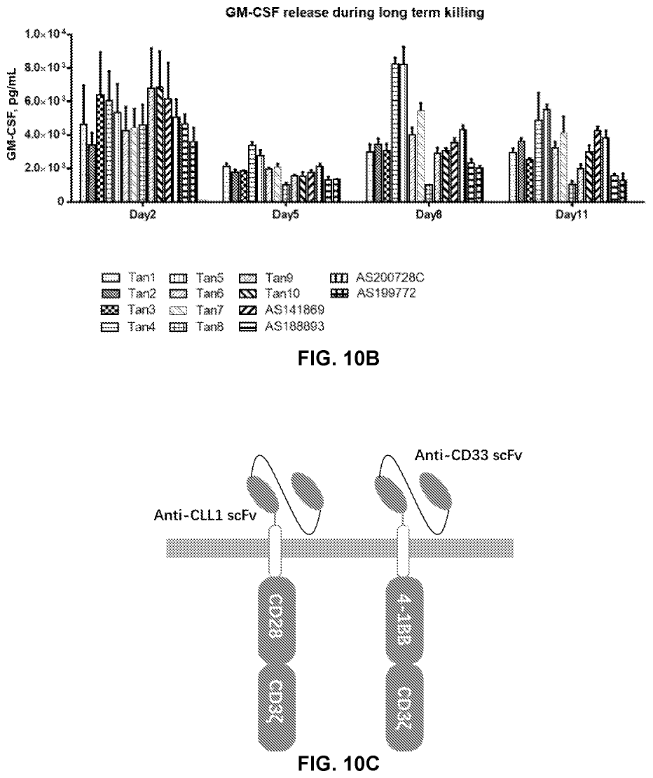

Another measure of effector T-cell activation and proliferation is the production of effector cytokines such as IFN-γ and GM-CSF. Supernatants from the long-term co-culture assay were collected to assess CAR-induced cytokine release. HTRF assays for IFN-γ (Cisbio, Cat #62HIFNGPEH) and GM-CSF (Cisbio, Cat #62HGMCSFPEG) were performed according to the manufacturer's manual. Results of a representative cytokines release assay are shown in FIG. 10A-10B. Tandem CAR-T cells released comparable levels of cytokines as the single-target CAR-T cells (AS138943, AS141567 CAR-T), when co-cultured with U937 cells in vitro.

### In Vivo Efficacy Evaluation in Mouse Xenograft Model

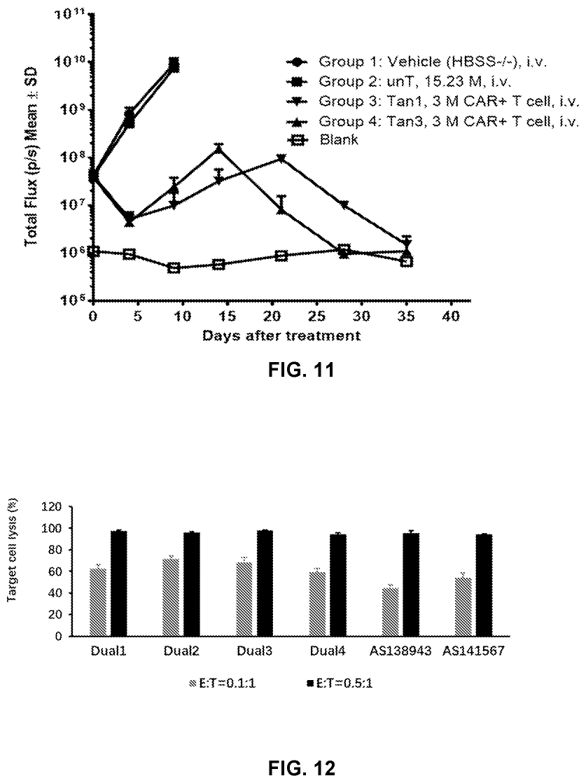

The in vivo efficacy of tandem CAR-T cells were evaluated in a U937-Luc xenograft mouse model as described in Example 2. As shown in FIG. 11, mice treated with tandem CAR-T cells were tumor free (BLI around 10⁶) after 5 weeks post injection, while mice with UnT cells or vehicle exhibited rapid tumor progression and had to be euthanized before the end of the experiment.

### Example 4. Generation and Evaluation of Anti-CD33/CLL1 Dual CAR Constructs

### Cloning of Anti-CD33/CLL1 Dual CAR Constructs

Exemplary dual CARs as shown in FIG. 10C were constructed by expressing two fully functional CARs against CD33 and CLL1 respectively. The CAR constructs were cloned into an expression vector with an EF1α promoter for expression. The sequences of exemplary dual CARs are shown in FIG. 15.

### Generation of Anti-CD33/CLL1 Dual CAR-T Cells

Lentiviruses encoding the dual CAR constructs as well as the individual CARs contained therein were prepared as described in Example 2. T lymphocytes were collected and transduced with the lentiviruses according to the protocol in Example 2.

### Evaluation of In Vitro Activity of Anti-CD33/CLL1 Dual CAR-T Cells

The anti-tumor activities of dual CAR-T cells were assessed using the in vitro LDH (lactate dehydrogenase) assay as described in Example 2. As shown in FIG. 12, in vitro cytotoxicity of dual CARs against U937 was comparable to that of anti-CLL1 CAR-T cells. These data indicates that dual CAR-T cells against two distinct targets (e.g., CD33 and CLL1) are more efficacious than single-target CAR-T cells for tumor elimination.

### Evaluation of Anti-CD33/CLL1 Dual CAR-T In Vivo Mouse Model

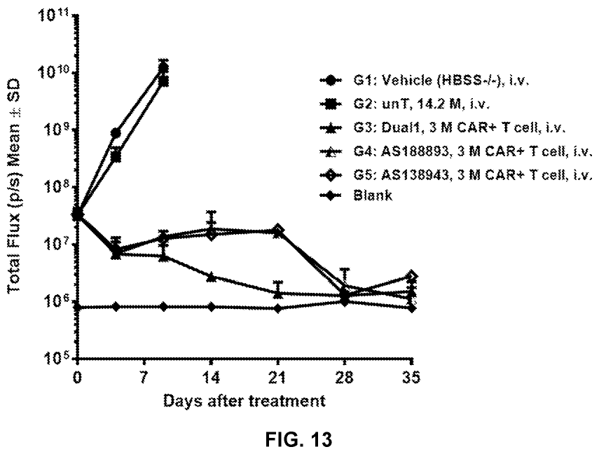

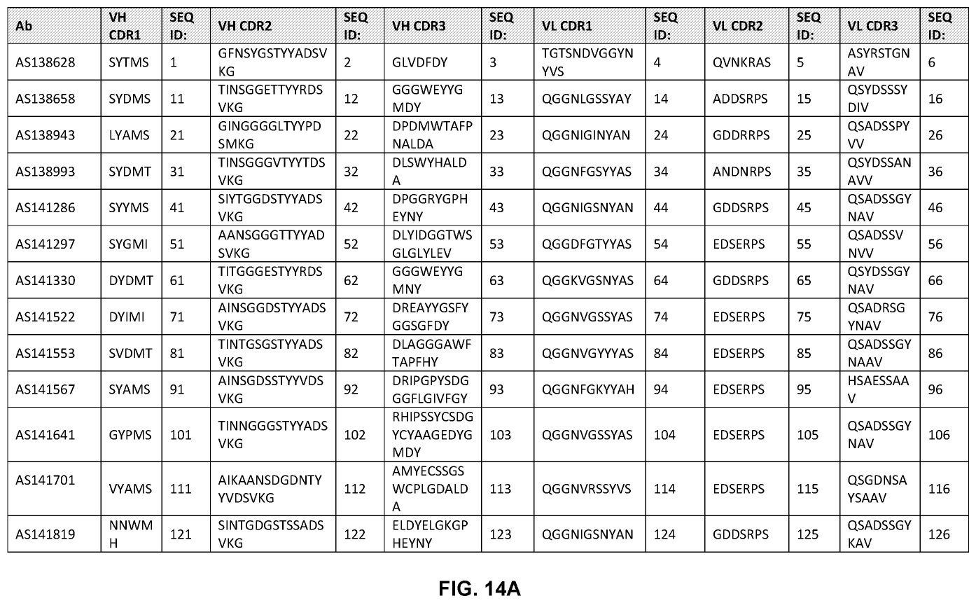

The in vivo efficacy of dual CAR-T cells were evaluated in a U937-Luc xenograft mouse model as described in Example 2. As shown in FIG. 13, mice treated with Dual1 CAR-T cells or single-target CAR-T cells were tumor free (BLI around 10⁶) after 3-4 weeks post injection, while mice with UnT cells or vehicle exhibited rapid tumor progression and had to be euthanized before the end of the experiment. Tumor growth in mice treated with dual CAR-T cells was significant slower than that in mice treated with single-target CAR T-cells (AS188893 CAR and AS138943 CAR). Combined with the results from Example 4, these data demonstrates that dual target CAR-T cells (tandem CARs or dual CARs) are more efficacious as tumor therapy than single-target CAR-T cells.

### OTHER EMBODIMENTS

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

What is claimed is:

1. An antibody or antigen-binding fragment thereof that binds to CLL1, comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs 1, 2, and 3 amino acid sequences are one of the following:
(1) the selected VH CDRs 1, 2, 3 amino acid sequences are set forth in SEQ ID NOs: 1, 2, and 3 respectively, and the selected VL CDRs 1, 2, 3 amino acid sequences are set forth in SEQ ID NOs: 4, 5, and 6 respectively;
(2) the selected VH CDRs 1, 2, 3 amino acid sequences are set forth in SEQ ID NOs: 11, 12, and 13 respectively, and the selected VL CDRs 1, 2, 3 amino acid sequences are set forth in SEQ ID NOs: 14, 15, and 16 respectively;
(3) the selected VH CDRs 1, 2, 3 amino acid sequences are set forth in SEQ ID NOs: 21, 22, and 23 respectively, and the selected VL CDRs 1, 2, 3 amino acid sequences are set forth in SEQ ID NOs: 24, 25, and 26 respectively;
(4) the selected VH CDRs 1, 2, 3 amino acid sequences are set forth in SEQ ID NOs: 31, 32, and 33 respectively, and the selected VL CDRs 1, 2, 3 amino acid sequences are set forth in SEQ ID NOs: 34, 35, and 36 respectively;
(5) the selected VH CDRs 1, 2, 3 amino acid sequences are set forth in SEQ ID NOs: 41, 42, and 43 respectively, and the selected VL CDRs 1, 2, 3 amino acid sequences are set forth in SEQ ID NOs: 44, 45, and 46 respectively;
(6) the selected VH CDRs 1, 2, 3 amino acid sequences are set forth in SEQ ID NOs: 51, 52, and 53 respectively, and the selected VL CDRs 1, 2, 3 amino acid sequences are set forth in SEQ ID NOs: 54, 55, and 56 respectively;
(7) the selected VH CDRs 1, 2, 3 amino acid sequences are set forth in SEQ ID NOs: 61, 62, and 63 respectively, and the selected VL CDRs 1, 2, 3 amino acid sequences are set forth in SEQ ID NOs: 64, 65, and 66 respectively;
(8) the selected VH CDRs 1, 2, 3 amino acid sequences are set forth in SEQ ID NOs: 71, 72, and 73 respectively, and the selected VL CDRs 1, 2, 3 amino acid sequences are set forth in SEQ ID NOs: 74, 75, and 76 respectively;
(9) the selected VH CDRs 1, 2, 3 amino acid sequences are set forth in SEQ ID NOs: 81, 82, and 83 respectively, and the selected VL CDRs 1, 2, 3 amino acid sequences are set forth in SEQ ID NOs: 84, 85, and 86 respectively;
(10) the selected VH CDRs 1, 2, 3 amino acid sequences are set forth in SEQ ID NOs: 91, 92, and 93 respectively, and the selected VL CDRs 1, 2, 3 amino acid sequences are set forth in SEQ ID NOs: 94, 95, and 96 respectively;
(11) the selected VH CDRs 1, 2, 3 amino acid sequences are set forth in SEQ ID NOs: 101, 102, and 103 respectively, and the selected VL CDRs 1, 2, 3 amino acid sequences are set forth in SEQ ID NOs: 104, 105, and 106 respectively;
(12) the selected VH CDRs 1, 2, 3 amino acid sequences are set forth in SEQ ID NOs: 111, 112, and 113 respectively, and the selected VL CDRs 1, 2, 3 amino acid sequences are set forth in SEQ ID NOs: 114, 115, and 116 respectively; and
(13) the selected VH CDRs 1, 2, 3 amino acid sequences are set forth in SEQ ID NOs: 121, 122, and 123 respectively, and the selected VL CDRs 1, 2, 3 amino acid sequences are set forth in SEQ ID NOs: 124, 125, and 126 respectively.

2. An antibody or antigen-binding fragment thereof that binds to CLL1, comprising a heavy chain variable region (VH) comprising an amino acid sequence that is at least 80%, 85%, 90%, 95% or 100% identical to a selected VH sequence, and a light chain variable region (VL) comprising an amino acid sequence that is at least 80%, 85%, 90%, 95% or 100% identical to a selected VL sequence, wherein the selected VH sequence and the selected VL sequence are one of the following:
(1) the selected VH sequence is SEQ ID NO: 10 and the selected VL sequence is SEQ ID NO: 9;
(2) the selected VH sequence is SEQ ID NO: 20, and the selected VL sequence is SEQ ID NO: 19;
(3) the selected VH sequence is SEQ ID NO: 30, and the selected VL sequence is SEQ ID NO: 29; and
(4) the selected VH sequence is SEQ ID NO: 40, and the selected VL sequence is SEQ ID NO: 39;
(5) the selected VH sequence is SEQ ID NO: 50, and the selected VL sequence is SEQ ID NO: 49;
(6) the selected VH sequence is SEQ ID NO: 60, and the selected VL sequence is SEQ ID NO: 59;
(7) the selected VH sequence is SEQ ID NO: 70, and the selected VL sequence is SEQ ID NO: 69;
(8) the selected VH sequence is SEQ ID NO: 80, and the selected VL sequence is SEQ ID NO: 79;
(9) the selected VH sequence is SEQ ID NO: 90, and the selected VL sequence is SEQ ID NO: 89;
(10) the selected VH sequence is SEQ ID NO: 100, and the selected VL sequence is SEQ ID NO: 99;
(11) the selected VH sequence is SEQ ID NO: 110, and the selected VL sequence is SEQ ID NO: 109;
(12) the selected VH sequence is SEQ ID NO: 120, and the selected VL sequence is SEQ ID NO: 119; and
(13) the selected VH sequence is SEQ ID NO: 130, and the selected VL sequence is SEQ ID NO: 129.

3. An antibody or antigen-binding fragment thereof that binds to CLL1, comprising
a heavy chain variable region (VH) comprising VH CDR1, VH CDR2, and VH CDR3 that are identical to VH CDR1, VH CDR2, and VH CDR3 of a selected VH sequence, and
a light chain variable region (VL) comprising VL CDR1, VL CDR2, and VL CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 10 and the selected VL sequence is SEQ ID NO: 9;
(2) the selected VH sequence is SEQ ID NO: 20, and the selected VL sequence is SEQ ID NO: 19;
(3) the selected VH sequence is SEQ ID NO: 30, and the selected VL sequence is SEQ ID NO: 29;
(4) the selected VH sequence is SEQ ID NO: 40, and the selected VL sequence is SEQ ID NO: 39;
(5) the selected VH sequence is SEQ ID NO: 50, and the selected VL sequence is SEQ ID NO: 49;
(6) the selected VH sequence is SEQ ID NO: 60, and the selected VL sequence is SEQ ID NO: 59;
(7) the selected VH sequence is SEQ ID NO: 70, and the selected VL sequence is SEQ ID NO: 69;
(8) the selected VH sequence is SEQ ID NO: 80, and the selected VL sequence is SEQ ID NO: 79;
(9) the selected VH sequence is SEQ ID NO: 90, and the selected VL sequence is SEQ ID NO: 89;
(10) the selected VH sequence is SEQ ID NO: 100, and the selected VL sequence is SEQ ID NO: 99;
(11) the selected VH sequence is SEQ ID NO: 110, and the selected VL sequence is SEQ ID NO: 109;
(12) the selected VH sequence is SEQ ID NO: 120, and the selected VL sequence is SEQ ID NO: 119; and
(13) the selected VH sequence is SEQ ID NO: 130, and the selected VL sequence is SEQ ID NO: 129.

4. The antibody or antigen-binding fragment thereof of any one of claims 1-3, wherein the antibody or antigen-binding fragment is a single-chain variable fragment (scFv).

5. The antibody or antigen-binding fragment thereof of any one of claims 1-4, wherein the antibody or antigen-binding fragment specifically binds to a human CLL1 peptide comprising a sequence that is at least 80%, 85%, 90%, 95%, or 100% identical to the amino acid sequence of SEQ ID NO: 147.

6. The antibody or antigen-binding fragment thereof of any one of claims 1-5, wherein the antibody or antigen-binding fragment specifically binds to the extracellular domain (ECD) of human CLL1.

7. The antibody or antigen-binding fragment thereof of any one of claims 1-6, wherein the antibody or antigen-binding fragment is a humanized antibody or antigen-binding fragment thereof.

8. The antibody or antigen-binding fragment thereof of any one of claims 1-7, wherein the antibody or antigen-binding fragment is a chimeric antibody or antigen-binding fragment thereof or a human antibody or antigen-binding fragment thereof.

9. An antibody or antigen-binding fragment thereof that cross-competes with the antibody or antigen-binding fragment thereof of any one of claims 1-8.

10. An antibody-drug conjugate comprising the antibody or antigen-binding fragment thereof of any one of claims 1-9 covalently bound to a therapeutic agent.

11. A pharmaceutical composition comprising the antibody or antigen-binding fragment thereof of any one of claims 1-9, or the antibody-drug conjugate of claim 10, and a pharmaceutically acceptable carrier.

12. A nucleic acid comprising a polynucleotide encoding the antibody or antigen-binding fragment thereof of any one of claims 1-9.

13. A vector comprising the nucleic acid of claim 12.

14. A cell comprising the vector of claim 13.

15. A method of producing an antibody or an antigen-binding fragment thereof, the method comprising
(a) culturing the cell of claim 14 under conditions sufficient for the cell to produce the antibody or the antigen-binding fragment thereof, and
(b) collecting the antibody or the antigen-binding fragment thereof produced by the cell.

16. An engineered receptor comprising the antigen-binding fragment thereof of any one of claims 1-9.

17. The engineered receptor of claim 16, wherein the engineered receptor further comprises a transmembrane region, and an intracellular signaling domain.

18. The engineered receptor of claim 16 or 17, wherein the engineered receptor is a chimeric antigen receptor (“CAR”).

19. The engineered receptor of any one of claims 16-18, wherein the engineered receptor further comprises a hinge region.

20. The engineered receptor of any one of claims 16-19, wherein the transmembrane region comprises a transmembrane region of CD4, CD8, and/or CD28, or a portion thereof.

21. The engineered receptor of any of claims 16-20, wherein the intracellular signaling domain comprises a primary intracellular signaling sequence of an immune effector cell.

22. The engineered receptor of claim 21, wherein the intracellular signaling domain is or comprises a functional signaling domain of CD3 zeta.

23. The engineered receptor of any of claims 16-22, wherein the intracellular signaling domain further comprises a costimulatory signaling domain.

24. The engineered receptor of claim 23, wherein the costimulatory signaling domain comprises a functional signaling domain from a protein selected from the group consisting of a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1, CD11a/CD18, 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, CD19a, and a CD83 ligand.

25. The engineered receptor of claim 24, wherein the costimulatory signaling domain comprises an intracellular signaling domain of 4-1BB and/or CD28.

26. The engineered receptor of any one of claims 16-25, wherein the engineered receptor comprises a signal peptide.

27. The engineered receptor of claim 26, wherein the signal peptide is at least 80%, 85%, 90%, 95% or 100% identical to SEQ ID NO: 146.

28. The engineered receptor of any one of claims 16-27, wherein the engineered receptor comprises an amino acid sequence set forth in any one of SEQ ID NOs: 7, 17, 27, 37, 47, 57, 67, 77, 87, 97, 107, 117, and 127, or an amino acid sequence that is at least 90%, 91%, 92%, 93%, 94%, 95%, 96%, 97%, 98%, 99% or 100% identical to any one of SEQ ID NOs: 7, 17, 27, 37, 47, 57, 67, 77, 87, 97, 107, 117, and 127.

29. The engineered receptor of claim 28, wherein the amino acid sequence is identical to any one of SEQ ID NOs: 7, 17, 27, 37, 47, 57, 67, 77, 87, 97, 107, 117, and 127.

30. An engineered receptor comprising:
(a) a first antigen-binding fragment thereof of any one of claims 1-9; and
(b) a second antigen-binding fragment thereof that binds to CD33.

31. The engineered receptor of claim 30, wherein the first antigen-binding fragment thereof and the second antigen-binding fragment thereof are connected via a linker.

32. The engineered receptor of claim 30 or 31, wherein the engineered receptor further comprises a transmembrane region, and an intracellular signaling domain.

33. The engineered receptor of any one of claims 30-32, wherein the engineered receptor is a chimeric antigen receptor (“CAR”).

34. The engineered receptor of any one of claims 30-33, wherein the engineered receptor further comprises a hinge region.

35. The engineered receptor of any one of claims 30-34, wherein the transmembrane region comprises a transmembrane region of CD4, CD8, and/or CD28, or a portion thereof.

36. The engineered receptor of any of claims 30-35, wherein the intracellular signaling domain comprises a primary intracellular signaling sequence of an immune effector cell.

37. The engineered receptor of claim 36, wherein the intracellular signaling domain is or comprises a functional signaling domain of CD3 zeta.

38. The engineered receptor of any of claims 30-37, wherein the intracellular signaling domain further comprises a costimulatory signaling domain.

39. The engineered receptor of claim 38, wherein the costimulatory signaling domain comprises a functional signaling domain from a protein selected from the group consisting of a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1, CD11a/CD18, 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, CD19a, and a CD83 ligand.

40. The engineered receptor of claim 39, wherein the costimulatory signaling domain comprises an intracellular signaling domain of 4-1BB and/or CD28.

41. The engineered receptor of any one of claims 30-40, wherein the engineered receptor comprises a signal peptide.

42. The engineered receptor of claim 41, wherein the signal peptide is at least 80%, 85%, 90%, 95% or 100% identical to SEQ ID NO: 146.

43. The engineered receptor of any one of claims 16-30, wherein the engineered receptor is a chimeric T cell receptor (“cTCR”).

44. The engineered receptor of claim 43, wherein the transmembrane domain is derived from the transmembrane domain of a TCR subunit selected from the group consisting of TCRα, TCRβ, TCRγ, TCRδ, CD3γ, CD3ε, and CD3δ.

45. The engineered receptor of claim 44, wherein the transmembrane domain is derived from the transmembrane domain of CD3ε.

46. The engineered receptor of any one of claims 43-45, wherein the intracellular signaling domain is derived from the intracellular signaling domain of a TCR subunit selected from the group consisting of TCRα, TCRβ, TCRγ, TCRδ, CD3γ, CD3ε, and CD3δ.

47. The engineered receptor of claim 46, wherein the intracellular signaling domain is derived from the intracellular signaling domain of CD3ε.

48. The engineered receptor of any one of claims 43-47, further comprising at least a portion of an extracellular domain of a TCR subunit.

49. The engineered receptor of claim 48, wherein the antigen binding fragment is fused to the N-terminus of CD3ε (“eTCR”).

50. A dual receptor system comprising:
(a) a first engineered receptor comprising a first antigen-binding fragment thereof of any one of claims 1-9 or is the engineered receptor of any one of claims 16-29; and
(b) a second engineered receptor comprising a second antigen-binding fragment thereof that binds to CD33.

51. The dual receptor system of claim 50, wherein each of the first engineered receptor and the second engineered receptor further comprises a transmembrane region, and an intracellular signaling domain.

52. The dual receptor system of claim 50 or 51, wherein the first engineered receptor and the second engineered receptor are both chimeric antigen receptors (“CARs”).

53. The dual receptor system of any one of claims 50-52, wherein each of the first engineered receptor and the second engineered receptor further comprises a hinge region.

54. The dual receptor system of any one of claims 50-53, wherein the transmembrane region comprises a transmembrane region of CD4, CD8, and/or CD28, or a portion thereof.

55. The dual receptor system of any one of claims 51-54, wherein the intracellular signaling domain comprises a primary intracellular signaling sequence of an immune effector cell.

56. The dual receptor system of claim 55, wherein the intracellular signaling domain is or comprises a functional signaling domain of CD3 zeta.

57. The dual receptor system of any one of claims 51-56, wherein the intracellular signaling domain further comprises a costimulatory signaling domain.

58. The dual receptor system of claim 57, wherein the costimulatory signaling domain comprises a functional signaling domain from a protein selected from the group consisting of a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1, CD11a/CD18, 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, CD19a, and a CD83 ligand.

59. The dual receptor system of claim 58, wherein the costimulatory signaling domain comprises an intracellular signaling domain of 4-1BB and/or CD28.

60. The dual receptor system of any one of claims 50-59, wherein each of the first engineered receptor and the second engineered receptor comprises a signal peptide.

61. The dual receptor system of claim 60, wherein the signal peptide is at least 80%, 85%, 90%, 95% or 100% identical to SEQ ID NO: 146.

62. The dual receptor system of any one of claims 50-61, wherein the second engineered receptor comprises an amino acid sequence set forth in SEQ ID NO: 159, 163, 167 or 171, or an amino acid sequence that is at least 90%, 91%, 92%, 93%, 94%, 95%, 96%, 97%, 98%, 99% or 100% identical to SEQ ID NO: 159, 163, 167 or 171.

63. The dual receptor system of any one of claims 50-61, wherein the first engineered receptor comprises an amino acid sequence set forth in SEQ ID NO: 27 or 97, or an amino acid sequence that is at least 90%, 91%, 92%, 93%, 94%, 95%, 96%, 97%, 98%, 99% or 100% identical to SEQ ID NO: 27 or 97.

64. The dual receptor system of any one of claims 50-61, wherein (1) the second engineered receptor comprises an amino acid sequence set forth in SEQ ID NO: 163, and the first engineered receptor comprises an amino acid sequence set forth in SEQ ID NO: 97; (2) the second engineered receptor comprises an amino acid sequence set forth in SEQ ID NO: 163, and the first engineered receptor comprises an amino acid sequence set forth in SEQ ID NO: 27; (3) the second engineered receptor comprises an amino acid sequence set forth in SEQ ID NO: 171, and the first engineered receptor comprises an amino acid sequence set forth in SEQ ID NO: 97; or (4) the second engineered receptor comprises an amino acid sequence set forth in SEQ ID NO: 171, and the first engineered receptor comprises an amino acid sequence set forth in SEQ ID NO: 27.

65. The dual receptor system of any one of claims 50-64, wherein the first engineered receptor and second engineered receptor are chimeric T cell receptors (“cTCRs”).

66. A polynucleotide encoding the engineered receptor or dual receptor system of any one of claims 16-65.

67. The polynucleotide of claim 66, encoding a polypeptide comprising an amino acid sequence of any one of SEQ ID NOs: 132-145, or an amino acid sequence that is at least 90%, 91%, 92%, 93%, 94%, 95%, 96%, 97%, 98%, 99% or 100% identical to SEQ ID NOs: 132-145.

68. A vector comprising the polynucleotide of claim 66 or 67.

69. The vector of claim 68, wherein the vector is a viral vector.

70. An engineered cell expressing the engineered receptor or dual receptor system of any one of claims 16-65.

71. The engineered cell of claim 70, comprising a polynucleotide encoding an amino acid sequence of any one of SEQ ID NOs: 132-145, or an amino acid sequence that is at least 90%, 91%, 92%, 93%, 94%, 95%, 96%, 97%, 98%, 99% or 100% identical to SEQ ID NOs: 132-145.

72. The engineered cell of claim 70 or 71, wherein the engineered cell is an immune cell.

73. The engineered cell of claim 72, wherein the immune cell is an NK cell or a T cell.

74. The engineered cell of claim 73, wherein the engineered cell is a T cell.

75. The engineered cell of claim 74, wherein the T cell is selected from the group consisting of cytotoxic T cell, a helper T cell, a natural killer T (NK-T) cell, an αβT cell and a γδT cell.

76. A method for producing an engineered cell, comprising introducing a vector of claim 68 or 69 into a cell in vitro or ex vivo.

77. The method of claim 76, wherein the vector is a viral vector and the introducing is carried out by transduction.

78. A method of treating cancer in a subject, comprising administering an effective amount of the antibody or antigen-binding fragment thereof of any one of claims 1-9, the antibody-drug conjugate of claim 10, the pharmaceutical composition of claim 11, or the engineered cell of any one of claims 70-75 to the subject.

79. The method of claim 78, wherein the cancer is acute myeloid leukemia (AML), chronic myelogenous leukemia (CML) or myelodysplastic syndromes (MDS).


SEQUENCE LISTING (201 sequences)

SEQ ID NO: 1 - protein, 5 aa, synthetic construct
SYTMS

SEQ ID NO: 2 - protein, 16 aa, synthetic construct
GFNSYGSTYYADSVKG

SEQ ID NO: 3 - protein, 7 aa, synthetic construct
GLVDFDY

SEQ ID NO: 4 - protein, 14 aa, synthetic construct
TGTSNDVGGYNYVS

SEQ ID NO: 5 - protein, 7 aa, synthetic construct
QVNKRAS

SEQ ID NO: 6 - protein, 10 aa, synthetic construct
ASYRSTGNAV

SEQ ID NO: 7 - protein, 480 aa, synthetic construct
MALPVTALLLPLALLLHAARPQSVLTQPSSVSGTPGQTVTISCTGTSNDVGGYNYVSWYQQLPGTAPKLLIYQVNKRASGIPDRFSGSKSGNTASMTISGLQSADEADYYCASYRSTGNAVFGGGTHLTVLGGGGSGGGGSGGGGSQVQLVQSGGGLVQPGGSLRLSCASSGFTFSSYTMSWVRQAPGKGLEWVSGFNSYGSTYYADSVKGRFTISRDNAKNTLYLQLNSLKTEDTAMYYCARGLVDFDYWGQGTQVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 240 aa, synthetic construct
QSVLTQPSSVSGTPGQTVTISCTGTSNDVGGYNYVSWYQQLPGTAPKLLIYQVNKRASGIPDRFSGSKSGNTASMTISGLQSADEADYYCASYRSTGNAVFGGGTHLTVLGGGGSGGGGSGGGGSQVQLVQSGGGLVQPGGSLRLSCASSGFTFSSYTMSWVRQAPGKGLEWVSGFNSYGSTYYADSVKGRFTISRDNAKNTLYLQLNSLKTEDTAMYYCARGLVDFDYWGQGTQVTVSS

SEQ ID NO: 9 - protein, 110 aa, synthetic construct
QSVLTQPSSVSGTPGQTVTISCTGTSNDVGGYNYVSWYQQLPGTAPKLLIYQVNKRASGIPDRFSGSKSGNTASMTISGLQSADEADYYCASYRSTGNAVFGGGTHLTVL

SEQ ID NO: 10 - protein, 115 aa, synthetic construct
QVQLVQSGGGLVQPGGSLRLSCASSGFTFSSYTMSWVRQAPGKGLEWVSGFNSYGSTYYADSVKGRFTISRDNAKNTLYLQLNSLKTEDTAMYYCARGLVDFDYWGQGTQVTVSS

SEQ ID NO: 11 - protein, 5 aa, synthetic construct
SYDMS

SEQ ID NO: 12 - protein, 17 aa, synthetic construct
TINSGGETTYYRDSVKG

SEQ ID NO: 13 - protein, 11 aa, synthetic construct
GGGWEYYGMDY

SEQ ID NO: 14 - protein, 11 aa, synthetic construct
QGGNLGSSYAY

SEQ ID NO: 15 - protein, 7 aa, synthetic construct
ADDSRPS

SEQ ID NO: 16 - protein, 11 aa, synthetic construct
QSYDSSSYDIV

SEQ ID NO: 17 - protein, 476 aa, synthetic construct
MALPVTALLLPLALLLHAARPSYELTQPSAVSVSLGQTARITCQGGNLGSSYAYWYQQKPGQAPVLVIYADDSRPSGIPERFSGSSSGGTATLTISGAQAEDEADYYCQSYDSSSYDIVFGGGTHLTVLGGGGSGGGGSGGGGSQVQLVQSGGSLRLSCAASGFTFSSYDMSWVRQAPGKGLEWVSTINSGGETTYYRDSVKGRFTISRDNAKNTLYLQLNSLKTEDTAMYYCAKGGGWEYYGMDYWGKGTLVTISSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 236 aa, synthetic construct
SYELTQPSAVSVSLGQTARITCQGGNLGSSYAYWYQQKPGQAPVLVIYADDSRPSGIPERFSGSSSGGTATLTISGAQAEDEADYYCQSYDSSSYDIVFGGGTHLTVLGGGGSGGGGSGGGGSQVQLVQSGGSLRLSCAASGFTFSSYDMSWVRQAPGKGLEWVSTINSGGETTYYRDSVKGRFTISRDNAKNTLYLQLNSLKTEDTAMYYCAKGGGWEYYGMDYWGKGTLVTISS

SEQ ID NO: 19 - protein, 108 aa, synthetic construct
SYELTQPSAVSVSLGQTARITCQGGNLGSSYAYWYQQKPGQAPVLVIYADDSRPSGIPERFSGSSSGGTATLTISGAQAEDEADYYCQSYDSSSYDIVFGGGTHLTVL

SEQ ID NO: 20 - protein, 113 aa, synthetic construct
QVQLVQSGGSLRLSCAASGFTFSSYDMSWVRQAPGKGLEWVSTINSGGETTYYRDSVKGRFTISRDNAKNTLYLQLNSLKTEDTAMYYCAKGGGWEYYGMDYWGKGTLVTISS

SEQ ID NO: 21 - protein, 5 aa, synthetic construct
LYAMS

SEQ ID NO: 22 - protein, 17 aa, synthetic construct
GINGGGGLTYYPDSMKG

SEQ ID NO: 23 - protein, 14 aa, synthetic construct
DPDMWTAFPNALDA

SEQ ID NO: 24 - protein, 11 aa, synthetic construct
QGGNIGINYAN

SEQ ID NO: 25 - protein, 7 aa, synthetic construct
GDDRRPS

SEQ ID NO: 26 - protein, 10 aa, synthetic construct
QSADSSPYVV

SEQ ID NO: 27 - protein, 485 aa, synthetic construct
MALPVTALLLPLALLLHAARPSYELTQPSAVSVSLGETARITCQGGNIGINYANWYQQKPGQAPVLVIYGDDRRPSGIPERFSGSISGGTATLTISGAQAEDEADYYCQSADSSPYVVFGGGTHLTVLGGGGSGGGGSGGGGSAVQLVESGGGLVQPGGSLRLSCTASGFNFGLYAMSWVRQAPGKGLEWVSGINGGGGLTYYPDSMKGRFTISRDVAKNTLYLELNSLKIEDTAMYYCAKDPDMWTAFPNALDAWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 245 aa, synthetic construct
SYELTQPSAVSVSLGETARITCQGGNIGINYANWYQQKPGQAPVLVIYGDDRRPSGIPERFSGSISGGTATLTISGAQAEDEADYYCQSADSSPYVVFGGGTHLTVLGGGGSGGGGSGGGGSAVQLVESGGGLVQPGGSLRLSCTASGFNFGLYAMSWVRQAPGKGLEWVSGINGGGGLTYYPDSMKGRFTISRDVAKNTLYLELNSLKIEDTAMYYCAKDPDMWTAFPNALDAWGQGTLVTVSS

SEQ ID NO: 29 - protein, 107 aa, synthetic construct
SYELTQPSAVSVSLGETARITCQGGNIGINYANWYQQKPGQAPVLVIYGDDRRPSGIPERFSGSISGGTATLTISGAQAEDEADYYCQSADSSPYVVFGGGTHLTVL

SEQ ID NO: 30 - protein, 123 aa, synthetic construct
AVQLVESGGGLVQPGGSLRLSCTASGFNFGLYAMSWVRQAPGKGLEWVSGINGGGGLTYYPDSMKGRFTISRDVAKNTLYLELNSLKIEDTAMYYCAKDPDMWTAFPNALDAWGQGTLVTVSS

SEQ ID NO: 31 - protein, 5 aa, synthetic construct
SYDMT

SEQ ID NO: 32 - protein, 17 aa, synthetic construct
TINSGGGVTYYTDSVKG

SEQ ID NO: 33 - protein, 10 aa, synthetic construct
DLSWYHALDA

SEQ ID NO: 34 - protein, 11 aa, synthetic construct
QGGNFGSYYAS

SEQ ID NO: 35 - protein, 7 aa, synthetic construct
ANDNRPS

SEQ ID NO: 36 - protein, 11 aa, synthetic construct
QSYDSSANAVV

SEQ ID NO: 37 - protein, 482 aa, synthetic construct
MALPVTALLLPLALLLHAARPSSELTQPAAVSVSLGETARITCQGGNFGSYYASWYQQKPGQAPVLVIHANDNRPSGIPERFSGSSSGGTVTLTISGAQAEDEADYYCQSYDSSANAVVFGGGTHLTVLGGGGSGGGGSGGGGSQVQLQESGGDLVQPGGSLRLSCAASGFTFSSYDMTWVRQAPGKGLEWVSTINSGGGVTYYTDSVKGRFTISRDNAKNTLYLQMNSLKPEDTAMYYCAADLSWYHALDAWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 242 aa, synthetic construct
SSELTQPAAVSVSLGETARITCQGGNFGSYYASWYQQKPGQAPVLVIHANDNRPSGIPERFSGSSSGGTVTLTISGAQAEDEADYYCQSYDSSANAVVFGGGTHLTVLGGGGSGGGGSGGGGSQVQLQESGGDLVQPGGSLRLSCAASGFTFSSYDMTWVRQAPGKGLEWVSTINSGGGVTYYTDSVKGRFTISRDNAKNTLYLQMNSLKPEDTAMYYCAADLSWYHALDAWGQGTLVTVSS

SEQ ID NO: 39 - protein, 108 aa, synthetic construct
SSELTQPAAVSVSLGETARITCQGGNFGSYYASWYQQKPGQAPVLVIHANDNRPSGIPERFSGSSSGGTVTLTISGAQAEDEADYYCQSYDSSANAVVFGGGTHLTVL

SEQ ID NO: 40 - protein, 119 aa, synthetic construct
QVQLQESGGDLVQPGGSLRLSCAASGFTFSSYDMTWVRQAPGKGLEWVSTINSGGGVTYYTDSVKGRFTISRDNAKNTLYLQMNSLKPEDTAMYYCAADLSWYHALDAWGQGTLVTVSS

SEQ ID NO: 41 - protein, 5 aa, synthetic construct
SYYMS

SEQ ID NO: 42 - protein, 17 aa, synthetic construct
SIYTGGDSTYYADSVKG

SEQ ID NO: 43 - protein, 13 aa, synthetic construct
DPGGRYGPHEYNY

SEQ ID NO: 44 - protein, 11 aa, synthetic construct
QGGNIGSNYAN

SEQ ID NO: 45 - protein, 7 aa, synthetic construct
GDDSRPS

SEQ ID NO: 46 - protein, 11 aa, synthetic construct
QSADSSGYNAV

SEQ ID NO: 47 - protein, 485 aa, synthetic construct
MALPVTALLLPLALLLHAARPSSELTQPSAVSVSLGETARITCQGGNIGSNYANWYQQKPGQAPVLVIYGDDSRPSGIPERFSGSSSGGTATLTISGAQAEDEADYYCQSADSSGYNAVFGGGTHLTVLGGGGSGGGGSGGGGSQVQLVQSGGGLVQPGGSLRLSCAASRFPDGSYYMSWVRQGPGKGLEWVSSIYTGGDSTYYADSVKGRFTISKDNAKNALYLQMNSLKPEDTAVYYCAADPGGRYGPHEYNYWGQGTQVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 245 aa, synthetic construct
SSELTQPSAVSVSLGETARITCQGGNIGSNYANWYQQKPGQAPVLVIYGDDSRPSGIPERFSGSSSGGTATLTISGAQAEDEADYYCQSADSSGYNAVFGGGTHLTVLGGGGSGGGGSGGGGSQVQLVQSGGGLVQPGGSLRLSCAASRFPDGSYYMSWVRQGPGKGLEWVSSIYTGGDSTYYADSVKGRFTISKDNAKNALYLQMNSLKPEDTAVYYCAADPGGRYGPHEYNYWGQGTQVTVSS

SEQ ID NO: 49 - protein, 108 aa, synthetic construct
SSELTQPSAVSVSLGETARITCQGGNIGSNYANWYQQKPGQAPVLVIYGDDSRPSGIPERFSGSSSGGTATLTISGAQAEDEADYYCQSADSSGYNAVFGGGTHLTVL

SEQ ID NO: 50 - protein, 122 aa, synthetic construct
QVQLVQSGGGLVQPGGSLRLSCAASRFPDGSYYMSWVRQGPGKGLEWVSSIYTGGDSTYYADSVKGRFTISKDNAKNALYLQMNSLKPEDTAVYYCAADPGGRYGPHEYNYWGQGTQVTVSS

SEQ ID NO: 51 - protein, 5 aa, synthetic construct
SYGMI

SEQ ID NO: 52 - protein, 17 aa, synthetic construct
AANSGGGTTYYADSVKG

SEQ ID NO: 53 - protein, 18 aa, synthetic construct
DLYIDGGTWSGLGLYLEV

SEQ ID NO: 54 - protein, 11 aa, synthetic construct
QGGDFGTYYAS

SEQ ID NO: 55 - protein, 7 aa, synthetic construct
EDSERPS

SEQ ID NO: 56 - protein, 10 aa, synthetic construct
QSADSSVNVV

SEQ ID NO: 57 - protein, 489 aa, synthetic construct
MALPVTALLLPLALLLHAARPSYELTQPSAVSVSLGETARITCQGGDFGTYYASWYQQKPGQAPVLVIYEDSERPSGIPERFSGSSSGGTATLTISGAQAEDEADYYCQSADSSVNVVFGGGTHLTVLGGGGSGGGGSGGGGSQVQLVQSGGGLVQPGGSLRLSCAASGFTFSSYGMIWVRQAPGKGLEWVSAANSGGGTTYYADSVKGRFTISRDNAKNTLYLQLNSLKTEDTAMYYCVKDLYIDGGTWSGLGLYLEVWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - protein, 249 aa, synthetic construct
SYELTQPSAVSVSLGETARITCQGGDFGTYYASWYQQKPGQAPVLVIYEDSERPSGIPERFSGSSSGGTATLTISGAQAEDEADYYCQSADSSVNVVFGGGTHLTVLGGGGSGGGGSGGGGSQVQLVQSGGGLVQPGGSLRLSCAASGFTFSSYGMIWVRQAPGKGLEWVSAANSGGGTTYYADSVKGRFTISRDNAKNTLYLQLNSLKTEDTAMYYCVKDLYIDGGTWSGLGLYLEVWGQGTLVTVSS

SEQ ID NO: 59 - protein, 107 aa, synthetic construct
SYELTQPSAVSVSLGETARITCQGGDFGTYYASWYQQKPGQAPVLVIYEDSERPSGIPERFSGSSSGGTATLTISGAQAEDEADYYCQSADSSVNVVFGGGTHLTVL

SEQ ID NO: 60 - protein, 127 aa, synthetic construct
QVQLVQSGGGLVQPGGSLRLSCAASGFTFSSYGMIWVRQAPGKGLEWVSAANSGGGTTYYADSVKGRFTISRDNAKNTLYLQLNSLKTEDTAMYYCVKDLYIDGGTWSGLGLYLEVWGQGTLVTVSS

SEQ ID NO: 61 - protein, 5 aa, synthetic construct
DYDMT

SEQ ID NO: 62 - protein, 17 aa, synthetic construct
TITGGGESTYYRDSVKG

SEQ ID NO: 63 - protein, 11 aa, synthetic construct
GGGWEYYGMNY

SEQ ID NO: 64 - protein, 11 aa, synthetic construct
QGGKVGSNYAS

SEQ ID NO: 65 - protein, 7 aa, synthetic construct
GDDSRPS

SEQ ID NO: 66 - protein, 11 aa, synthetic construct
QSYDSSGYNAV

SEQ ID NO: 67 - protein, 476 aa, synthetic construct
MALPVTALLLPLALLLHAARPSSELTQPSAVSVSLGETARITCQGGKVGSNYASWYQQKPGQAPVLVIYGDDSRPSGIPERFSGSSSGGTATLTISGAQAEDEADYYCQSYDSSGYNAVFGGGTHLTVLGGGGSGGGGSGGGGSQVKLEQPGGSLRLSCAASGFTFSDYDMTWVRQAPGKGLEWISTITGGGESTYYRDSVKGRATISRDNAKNTLYLQLNSLKTEDTAMYYCARGGGWEYYGMNYWGKGTLVTISSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 236 aa, synthetic construct
SSELTQPSAVSVSLGETARITCQGGKVGSNYASWYQQKPGQAPVLVIYGDDSRPSGIPERFSGSSSGGTATLTISGAQAEDEADYYCQSYDSSGYNAVFGGGTHLTVLGGGGSGGGGSGGGGSQVKLEQPGGSLRLSCAASGFTFSDYDMTWVRQAPGKGLEWISTITGGGESTYYRDSVKGRATISRDNAKNTLYLQLNSLKTEDTAMYYCARGGGWEYYGMNYWGKGTLVTISS

SEQ ID NO: 69 - protein, 108 aa, synthetic construct
SSELTQPSAVSVSLGETARITCQGGKVGSNYASWYQQKPGQAPVLVIYGDDSRPSGIPERFSGSSSGGTATLTISGAQAEDEADYYCQSYDSSGYNAVFGGGTHLTVL

SEQ ID NO: 70 - protein, 113 aa, synthetic construct
QVKLEQPGGSLRLSCAASGFTFSDYDMTWVRQAPGKGLEWISTITGGGESTYYRDSVKGRATISRDNAKNTLYLQLNSLKTEDTAMYYCARGGGWEYYGMNYWGKGTLVTISS

SEQ ID NO: 71 - protein, 5 aa, synthetic construct
DYIMI

SEQ ID NO: 72 - protein, 17 aa, synthetic construct
AINSGGDSTYYADSVKG

SEQ ID NO: 73 - protein, 17 aa, synthetic construct
DREAYYGSFYGGSGFDY

SEQ ID NO: 74 - protein, 11 aa, synthetic construct
QGGNVGSSYAS

SEQ ID NO: 75 - protein, 7 aa, synthetic construct
EDSERPS

SEQ ID NO: 76 - protein, 11 aa, synthetic construct
QSADRSGYNAV

SEQ ID NO: 77 - protein, 489 aa, synthetic construct
MALPVTALLLPLALLLHAARPSSELTQPSAVSVSLGETARITCQGGNVGSSYASWYQQKPGQAPVLVIYEDSERPSGIPERFSGSSSGGTATLTISRAQAEDEADYYCQSADRSGYNAVFGGGTHLTVLGGGGSGGGGSGGGGSQVQLVQSGGGLVQPGGSLRLSCAASGFTFGDYIMIWVRQAPGKGLEWVSAINSGGDSTYYADSVKGRFTISRDNAKNTLYLQLNSLKTQDTAIYYCAKDREAYYGSFYGGSGFDYWGQGTQVTISSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 78 - protein, 249 aa, synthetic construct
SSELTQPSAVSVSLGETARITCQGGNVGSSYASWYQQKPGQAPVLVIYEDSERPSGIPERFSGSSSGGTATLTISRAQAEDEADYYCQSADRSGYNAVFGGGTHLTVLGGGGSGGGGSGGGGSQVQLVQSGGGLVQPGGSLRLSCAASGFTFGDYIMIWVRQAPGKGLEWVSAINSGGDSTYYADSVKGRFTISRDNAKNTLYLQLNSLKTQDTAIYYCAKDREAYYGSFYGGSGFDYWGQGTQVTISS

SEQ ID NO: 79 - protein, 108 aa, synthetic construct
SSELTQPSAVSVSLGETARITCQGGNVGSSYASWYQQKPGQAPVLVIYEDSERPSGIPERFSGSSSGGTATLTISRAQAEDEADYYCQSADRSGYNAVFGGGTHLTVL

SEQ ID NO: 80 - protein, 126 aa, synthetic construct
QVQLVQSGGGLVQPGGSLRLSCAASGFTFGDYIMIWVRQAPGKGLEWVSAINSGGDSTYYADSVKGRFTISRDNAKNTLYLQLNSLKTQDTAIYYCAKDREAYYGSFYGGSGFDYWGQGTQVTISS

SEQ ID NO: 81 - protein, 5 aa, synthetic construct
SVDMT

SEQ ID NO: 82 - protein, 17 aa, synthetic construct
TINTGSGSTYYADSVKG

SEQ ID NO: 83 - protein, 15 aa, synthetic construct
DLAGGGAWFTAPFHY

SEQ ID NO: 84 - protein, 11 aa, synthetic construct
QGGNVGYYYAS

SEQ ID NO: 85 - protein, 7 aa, synthetic construct
EDSERPS

SEQ ID NO: 86 - protein, 12 aa, synthetic construct
QSADSSGYNAAV

SEQ ID NO: 87 - protein, 488 aa, synthetic construct
MALPVTALLLPLALLLHAARPSYELTQPSAVSVSLGETARITCQGGNVGYYYASWYQQKPGQAPVLVIYEDSERPSGIPERFSGSSSGGTATLTISGAQAEDEADYYCQSADSSGYNAAVFGGGTHLTVLGGGGSGGGGSGGGGSQVQLVESGGGLVQPGGSLRLSCAASGFTFSSVDMTWVRQAPGKGLEWVSTINTGSGSTYYADSVKGRFTISRDNAKNTLYLQMNSLKTEDTAVYYCATDLAGGGAWFTAPFHYWGQGTQVTISSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 88 - protein, 248 aa, synthetic construct
SYELTQPSAVSVSLGETARITCQGGNVGYYYASWYQQKPGQAPVLVIYEDSERPSGIPERFSGSSSGGTATLTISGAQAEDEADYYCQSADSSGYNAAVFGGGTHLTVLGGGGSGGGGSGGGGSQVQLVESGGGLVQPGGSLRLSCAASGFTFSSVDMTWVRQAPGKGLEWVSTINTGSGSTYYADSVKGRFTISRDNAKNTLYLQMNSLKTEDTAVYYCATDLAGGGAWFTAPFHYWGQGTQVTISS

SEQ ID NO: 89 - protein, 109 aa, synthetic construct
SYELTQPSAVSVSLGETARITCQGGNVGYYYASWYQQKPGQAPVLVIYEDSERPSGIPERFSGSSSGGTATLTISGAQAEDEADYYCQSADSSGYNAAVFGGGTHLTVL

SEQ ID NO: 90 - protein, 124 aa, synthetic construct
QVQLVESGGGLVQPGGSLRLSCAASGFTFSSVDMTWVRQAPGKGLEWVSTINTGSGSTYYADSVKGRFTISRDNAKNTLYLQMNSLKTEDTAVYYCATDLAGGGAWFTAPFHYWGQGTQVTISS

SEQ ID NO: 91 - protein, 5 aa, synthetic construct
SYAMS

SEQ ID NO: 92 - protein, 17 aa, synthetic construct
AINSGDSSTYYVDSVKG

SEQ ID NO: 93 - protein, 20 aa, synthetic construct
DRIPGPYSDGGGFLGIVFGY

SEQ ID NO: 94 - protein, 11 aa, synthetic construct
QGGNFGKYYAH

SEQ ID NO: 95 - protein, 7 aa, synthetic construct
EDSERPS

SEQ ID NO: 96 - protein, 9 aa, synthetic construct
HSAESSAAV

SEQ ID NO: 97 - protein, 490 aa, synthetic construct
MALPVTALLLPLALLLHAARPSSELTQPSAVSVSLGQTARITCQGGNFGKYYAHWYQQKPGQAPVLVIYEDSERPSGIPERFSGSSSGDTATLTISGAQAEDEGDYYCHSAESSAAVFGGGTHLTVLGGGGSGGGGSGGGGSQVQLVQSGGGLVQPGGSLRLSCAASGFTFSSYAMSWVRQAPGKGLEWVSAINSGDSSTYYVDSVKGRFTISRDNAKNTLYLQLNSLKTEDTAMYYCAKDRIPGPYSDGGGFLGIVFGYWGQGTQVTISSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 98 - protein, 250 aa, synthetic construct
SSELTQPSAVSVSLGQTARITCQGGNFGKYYAHWYQQKPGQAPVLVIYEDSERPSGIPERFSGSSSGDTATLTISGAQAEDEGDYYCHSAESSAAVFGGGTHLTVLGGGGSGGGGSGGGGSQVQLVQSGGGLVQPGGSLRLSCAASGFTFSSYAMSWVRQAPGKGLEWVSAINSGDSSTYYVDSVKGRFTISRDNAKNTLYLQLNSLKTEDTAMYYCAKDRIPGPYSDGGGFLGIVFGYWGQGTQVTISS

SEQ ID NO: 99 - protein, 106 aa, synthetic construct
SSELTQPSAVSVSLGQTARITCQGGNFGKYYAHWYQQKPGQAPVLVIYEDSERPSGIPERFSGSSSGDTATLTISGAQAEDEGDYYCHSAESSAAVFGGGTHLTVL

SEQ ID NO: 100 - protein, 129 aa, synthetic construct
QVQLVQSGGGLVQPGGSLRLSCAASGFTFSSYAMSWVRQAPGKGLEWVSAINSGDSSTYYVDSVKGRFTISRDNAKNTLYLQLNSLKTEDTAMYYCAKDRIPGPYSDGGGFLGIVFGYWGQGTQVTISS

SEQ ID NO: 101 - protein, 5 aa, synthetic construct
GYPMS

SEQ ID NO: 102 - protein, 17 aa, synthetic construct
TINNGGGSTYYADSVKG

SEQ ID NO: 103 - protein, 24 aa, synthetic construct
RHIPSSYCSDGYCYAAGEDYGMDY

SEQ ID NO: 104 - protein, 11 aa, synthetic construct
QGGNVGSSYAS

SEQ ID NO: 105 - protein, 7 aa, synthetic construct
EDSERPS

SEQ ID NO: 106 - protein, 11 aa, synthetic construct
QSADSSGYNAV

SEQ ID NO: 107 - protein, 496 aa, synthetic construct
MALPVTALLLPLALLLHAARPSSELTQPSAVSVSLGETARITCQGGNVGSSYASWYQQKPGQAPVLVIYEDSERPSGIPERFSGSSSGGTATLTISGAQAEDEADYYCQSADSSGYNAVFGGGTHLTVLGGGGSGGGGSGGGGSAVQLVESGGGLVQPGGSLRLSCAASGFTFSGYPMSWVRQAPGKALEWVSTINNGGGSTYYADSVKGRFTISRDNAKNTLYLQLNSLKTEDTAMYYCAKRHIPSSYCSDGYCYAAGEDYGMDYWGKGTLVTISSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 108 - protein, 256 aa, synthetic construct
SSELTQPSAVSVSLGETARITCQGGNVGSSYASWYQQKPGQAPVLVIYEDSERPSGIPERFSGSSSGGTATLTISGAQAEDEADYYCQSADSSGYNAVFGGGTHLTVLGGGGSGGGGSGGGGSAVQLVESGGGLVQPGGSLRLSCAASGFTFSGYPMSWVRQAPGKALEWVSTINNGGGSTYYADSVKGRFTISRDNAKNTLYLQLNSLKTEDTAMYYCAKRHIPSSYCSDGYCYAAGEDYGMDYWGKGTLVTISS

SEQ ID NO: 109 - protein, 108 aa, synthetic construct
SSELTQPSAVSVSLGETARITCQGGNVGSSYASWYQQKPGQAPVLVIYEDSERPSGIPERFSGSSSGGTATLTISGAQAEDEADYYCQSADSSGYNAVFGGGTHLTVL

SEQ ID NO: 110 - protein, 133 aa, synthetic construct
AVQLVESGGGLVQPGGSLRLSCAASGFTFSGYPMSWVRQAPGKALEWVSTINNGGGSTYYADSVKGRFTISRDNAKNTLYLQLNSLKTEDTAMYYCAKRHIPSSYCSDGYCYAAGEDYGMDYWGKGTLVTISS

SEQ ID NO: 111 - protein, 5 aa, synthetic construct
VYAMS

SEQ ID NO: 112 - protein, 20 aa, synthetic construct
AIKAANSDGDNTYYVDSVKG

SEQ ID NO: 113 - protein, 19 aa, synthetic construct
AMYECSSGSWCPLGDALDA

SEQ ID NO: 114 - protein, 11 aa, synthetic construct
QGGNVRSSYVS

SEQ ID NO: 115 - protein, 7 aa, synthetic construct
EDSERPS

SEQ ID NO: 116 - protein, 12 aa, synthetic construct
QSGDNSAYSAAV

SEQ ID NO: 117 - protein, 495 aa, synthetic construct
MALPVTALLLPLALLLHAARPSSELTQPSAVSVSLGETARITCQGGNVRSSYVSWYQQKPGQAPVLVIYEDSERPSGIPERFSGSSSGGTATLTISGAQAEDEADYYCQSGDNSAYSAAVFGGGTHLTVLGGGGSGGGGSGGGGSQVQLVQSGGGLVQPGGSLRLSCAASGFTFSVYAMSWVRQAPGKGLEWVSAIKAANSDGDNTYYVDSVKGRFTISRDNAKNTLYLQLNSLKTEDTAIYYCAKAMYECSSGSWCPLGDALDAWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 118 - protein, 255 aa, synthetic construct
SSELTQPSAVSVSLGETARITCQGGNVRSSYVSWYQQKPGQAPVLVIYEDSERPSGIPERFSGSSSGGTATLTISGAQAEDEADYYCQSGDNSAYSAAVFGGGTHLTVLGGGGSGGGGSGGGGSQVQLVQSGGGLVQPGGSLRLSCAASGFTFSVYAMSWVRQAPGKGLEWVSAIKAANSDGDNTYYVDSVKGRFTISRDNAKNTLYLQLNSLKTEDTAIYYCAKAMYECSSGSWCPLGDALDAWGQGTLVTVSS

SEQ ID NO: 119 - protein, 109 aa, synthetic construct
SSELTQPSAVSVSLGETARITCQGGNVRSSYVSWYQQKPGQAPVLVIYEDSERPSGIPERFSGSSSGGTATLTISGAQAEDEADYYCQSGDNSAYSAAVFGGGTHLTVL

SEQ ID NO: 120 - protein, 131 aa, synthetic construct
QVQLVQSGGGLVQPGGSLRLSCAASGFTFSVYAMSWVRQAPGKGLEWVSAIKAANSDGDNTYYVDSVKGRFTISRDNAKNTLYLQLNSLKTEDTAIYYCAKAMYECSSGSWCPLGDALDAWGQGTLVTVSS

SEQ ID NO: 121 - protein, 5 aa, synthetic construct
NNWMH

SEQ ID NO: 122 - protein, 17 aa, synthetic construct
SINTGDGSTSSADSVKG

SEQ ID NO: 123 - protein, 15 aa, synthetic construct
ELDYELGKGPHEYNY

SEQ ID NO: 124 - protein, 11 aa, synthetic construct
QGGNIGSNYAN

SEQ ID NO: 125 - protein, 7 aa, synthetic construct
GDDSRPS

SEQ ID NO: 126 - protein, 11 aa, synthetic construct
QSADSSGYKAV

SEQ ID NO: 127 - protein, 487 aa, synthetic construct
MALPVTALLLPLALLLHAARPSYELTQPSAVSVSLGETARITCQGGNIGSNYANWYQQKPDQAPVLVIYGDDSRPSGIPERFSGSSSGGTATLTISGAQAEDETDYYCQSADSSGYKAVFGGGTHLTVLGGGGSGGGGSGGGGSEVQLVQSGGGLVQPGGSLRLSCATSGFTFSNNWMHWVRQAPGKGLEWVSSINTGDGSTSSADSVKGRFTISRDNTKNILYLQMNSLKSEDTALYYCAKELDYELGKGPHEYNYWGQGTQVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 128 - protein, 247 aa, synthetic construct
SYELTQPSAVSVSLGETARITCQGGNIGSNYANWYQQKPDQAPVLVIYGDDSRPSGIPERFSGSSSGGTATLTISGAQAEDETDYYCQSADSSGYKAVFGGGTHLTVLGGGGSGGGGSGGGGSEVQLVQSGGGLVQPGGSLRLSCATSGFTFSNNWMHWVRQAPGKGLEWVSSINTGDGSTSSADSVKGRFTISRDNTKNILYLQMNSLKSEDTALYYCAKELDYELGKGPHEYNYWGQGTQVTVSS

SEQ ID NO: 129 - protein, 108 aa, synthetic construct
SYELTQPSAVSVSLGETARITCQGGNIGSNYANWYQQKPDQAPVLVIYGDDSRPSGIPERFSGSSSGGTATLTISGAQAEDETDYYCQSADSSGYKAVFGGGTHLTVL

SEQ ID NO: 130 - protein, 124 aa, synthetic construct
EVQLVQSGGGLVQPGGSLRLSCATSGFTFSNNWMHWVRQAPGKGLEWVSSINTGDGSTSSADSVKGRFTISRDNTKNILYLQMNSLKSEDTALYYCAKELDYELGKGPHEYNYWGQGTQVTVSS

SEQ ID NO: 131 - protein, 370 aa, synthetic construct
MALPVTALLLPLALLLHAARPEVQLAESGGGLVQAGGSLRLSCTASGFTFDDYVMGWFRQAPGKEREGVSCISWSGDTTYYADSVKGRFTASRDNAKNTLYLQMNSLKPEDTAMYYCAADQGKCSLASAEPDDMDYWGRGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 132 - protein, 753 aa, synthetic construct
MALPVTALLLPLALLLHAARPSYELTQPSAVSVSLGQTARITCQGGNLGTSYVHWYQQKPGQAPVLVIYGDDSRPSGIPERFSGSSSGGTATLTISGAQAEDEADYYCQSYDSSSSVFGGGTHLTVLGGGGSGGGGSGGGGSQVQLQESGGGLVQPGGSLRLSCAASGFTFSNNWMYWVRQAPGKGLEWVSSIATGDGSTNSADSVKGRFTISRDNTKNMLYLQMNSLKSEDTALYYCAKGVNIDHDVGAGDYWGQGTQVTVSSGGGGSGGGGSGGGGSGGGGSGGGGSSYELTQPSAVSVSLGETARITCQGGNIGINYANWYQQKPGQAPVLVIYGDDRRPSGIPERFSGSISGGTATLTISGAQAEDEADYYCQSADSSPYVVFGGGTHLTVLGGGGSGGGGSGGGGSAVQLVESGGGLVQPGGSLRLSCTASGFNFGLYAMSWVRQAPGKGLEWVSGINGGGGLTYYPDSMKGRFTISRDVAKNTLYLELNSLKIEDTAMYYCAKDPDMWTAFPNALDAWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 133 - protein, 734 aa, synthetic construct
MALPVTALLLPLALLLHAARPSYELTQPSAVSVSLGQTARITCQGGNLGTSYVHWYQQKPGQAPVLVIYGDDSRPSGIPERFSGSSSGGTATLTISGAQAEDEADYYCQSYDSSSSVFGGGTHLTVLGGGGSGGGGSGGGGSQVQLQESGGGLVQPGGSLRLSCAASGFTFSNNWMYWVRQAPGKGLEWVSSIATGDGSTNSADSVKGRFTISRDNTKNMLYLQMNSLKSEDTALYYCAKGVNIDHDVGAGDYWGQGTQVTVSSSGGGGSSYELTQPSAVSVSLGETARITCQGGNIGINYANWYQQKPGQAPVLVIYGDDRRPSGIPERFSGSISGGTATLTISGAQAEDEADYYCQSADSSPYVVFGGGTHLTVLGGGGSGGGGSGGGGSAVQLVESGGGLVQPGGSLRLSCTASGFNFGLYAMSWVRQAPGKGLEWVSGINGGGGLTYYPDSMKGRFTISRDVAKNTLYLELNSLKIEDTAMYYCAKDPDMWTAFPNALDAWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 134 - protein, 726 aa, synthetic construct
MALPVTALLLPLALLLHAARPSYELTQPSAVSVSLGQTARITCQGGNLGTSYVHWYQQKPGQAPVLVIYGDDSRPSGIPERFSGSSSGGTATLTISGAQAEDEADYYCQSYDSSSSVFGGGTHLTVLGGGGSAVQLVESGGGLVQPGGSLRLSCTASGFNFGLYAMSWVRQAPGKGLEWVSGINGGGGLTYYPDSMKGRFTISRDVAKNTLYLELNSLKIEDTAMYYCAKDPDMWTAFPNALDAWGQGTLVTVSSGSTSGSGKPGSGEGSTKGSYELTQPSAVSVSLGETARITCQGGNIGINYANWYQQKPGQAPVLVIYGDDRRPSGIPERFSGSISGGTATLTISGAQAEDEADYYCQSADSSPYVVFGGGTHLTVLGGGGSQVQLQESGGGLVQPGGSLRLSCAASGFTFSNNWMYWVRQAPGKGLEWVSSIATGDGSTNSADSVKGRFTISRDNTKNMLYLQMNSLKSEDTALYYCAKGVNIDHDVGAGDYWGQGTQVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 135 - protein, 759 aa, synthetic construct
MALPVTALLLPLALLLHAARPYELTQSPSVSVALRQAAKITCRGDNIETKYAYWYQQKPGQSPLLVIYKDDNRPSGIPERFSGSNSGNMATLTISGAQAEDEADYYCQVWDSSANWVFGGGTHLTVLGGGGSGGGGSGGGGSQVQLQESGGGLVQPGGSLRLSCAASGFTFSSYWMYWVRQAPGKGLEWVSAMNTGGGSTYYGDSVKGRFTVSRDNAKNTLALQMNSLKPEDTALYYCARSWGGMYSGGYYEYSGMDYWGKGTLVTVSSSGGGGSGGGGSGGGGSGGGGSGGGGSSYELTQPSAVSVSLGETARITCQGGNIGINYANWYQQKPGQAPVLVIYGDDRRPSGIPERFSGSISGGTATLTISGAQAEDEADYYCQSADSSPYVVFGGGTHLTVLGGGGSGGGGSGGGGSAVQLVESGGGLVQPGGSLRLSCTASGFNFGLYAMSWVRQAPGKGLEWVSGINGGGGLTYYPDSMKGRFTISRDVAKNTLYLELNSLKIEDTAMYYCAKDPDMWTAFPNALDAWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 136 - protein, 740 aa, synthetic construct
MALPVTALLLPLALLLHAARPYELTQSPSVSVALRQAAKITCRGDNIETKYAYWYQQKPGQSPLLVIYKDDNRPSGIPERFSGSNSGNMATLTISGAQAEDEADYYCQVWDSSANWVFGGGTHLTVLGGGGSGGGGSGGGGSQVQLQESGGGLVQPGGSLRLSCAASGFTFSSYWMYWVRQAPGKGLEWVSAMNTGGGSTYYGDSVKGRFTVSRDNAKNTLALQMNSLKPEDTALYYCARSWGGMYSGGYYEYSGMDYWGKGTLVTVSSSSGGGGSSYELTQPSAVSVSLGETARITCQGGNIGINYANWYQQKPGQAPVLVIYGDDRRPSGIPERFSGSISGGTATLTISGAQAEDEADYYCQSADSSPYVVFGGGTHLTVLGGGGSGGGGSGGGGSAVQLVESGGGLVQPGGSLRLSCTASGFNFGLYAMSWVRQAPGKGLEWVSGINGGGGLTYYPDSMKGRFTISRDVAKNTLYLELNSLKIEDTAMYYCAKDPDMWTAFPNALDAWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 137 - protein, 732 aa, synthetic construct
MALPVTALLLPLALLLHAARPYELTQSPSVSVALRQAAKITCRGDNIETKYAYWYQQKPGQSPLLVIYKDDNRPSGIPERFSGSNSGNMATLTISGAQAEDEADYYCQVWDSSANWVFGGGTHLTVLGGGGSAVQLVESGGGLVQPGGSLRLSCTASGFNFGLYAMSWVRQAPGKGLEWVSGINGGGGLTYYPDSMKGRFTISRDVAKNTLYLELNSLKIEDTAMYYCAKDPDMWTAFPNALDAWGQGTLVTVSSGSTSGSGKPGSGEGSTKGSYELTQPSAVSVSLGETARITCQGGNIGINYANWYQQKPGQAPVLVIYGDDRRPSGIPERFSGSISGGTATLTISGAQAEDEADYYCQSADSSPYVVFGGGTHLTVLGGGGSQVQLQESGGGLVQPGGSLRLSCAASGFTFSSYWMYWVRQAPGKGLEWVSAMNTGGGSTYYGDSVKGRFTVSRDNAKNTLALQMNSLKPEDTALYYCARSWGGMYSGGYYEYSGMDYWGKGTLVTVSSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 138 - protein, 760 aa, synthetic construct
MALPVTALLLPLALLLHAARPQAVVTQLSSMSGSPGQTVTITCTGSSSNIGGGYYLSWYQQLPGTAPKLLIYNVNNRASGVPNRFSGSKTGSLASLTITGLQAEDEADYYCGSYDESLSYVFGGGTHLTVLGGGGSGGGGSGGGGSQVQLVESGGGLVQPGGSLRVSCAASGFTFKNYYMGWVRQAPGKGLEWISGINSGGEVTTYADSVKDRFTISRDNAKNTLYLQMNSLKPDDTALYYCAGGEGSSWYPDYYYGNGYWGEGTLITVSSGGGGSGGGGSGGGGSGGGGSGGGGSSYELTQPSAVSVSLGETARITCQGGNIGINYANWYQQKPGQAPVLVIYGDDRRPSGIPERFSGSISGGTATLTISGAQAEDEADYYCQSADSSPYVVFGGGTHLTVLGGGGSGGGGSGGGGSAVQLVESGGGLVQPGGSLRLSCTASGFNFGLYAMSWVRQAPGKGLEWVSGINGGGGLTYYPDSMKGRFTISRDVAKNTLYLELNSLKIEDTAMYYCAKDPDMWTAFPNALDAWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 139 - protein, 741 aa, synthetic construct
MALPVTALLLPLALLLHAARPQAVVTQLSSMSGSPGQTVTITCTGSSSNIGGGYYLSWYQQLPGTAPKLLIYNVNNRASGVPNRFSGSKTGSLASLTITGLQAEDEADYYCGSYDESLSYVFGGGTHLTVLGGGGSGGGGSGGGGSQVQLVESGGGLVQPGGSLRVSCAASGFTFKNYYMGWVRQAPGKGLEWISGINSGGEVTTYADSVKDRFTISRDNAKNTLYLQMNSLKPDDTALYYCAGGEGSSWYPDYYYGNGYWGEGTLITVSSSGGGGSSYELTQPSAVSVSLGETARITCQGGNIGINYANWYQQKPGQAPVLVIYGDDRRPSGIPERFSGSISGGTATLTISGAQAEDEADYYCQSADSSPYVVFGGGTHLTVLGGGGSGGGGSGGGGSAVQLVESGGGLVQPGGSLRLSCTASGFNFGLYAMSWVRQAPGKGLEWVSGINGGGGLTYYPDSMKGRFTISRDVAKNTLYLELNSLKIEDTAMYYCAKDPDMWTAFPNALDAWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 140 - protein, 733 aa, synthetic construct
MALPVTALLLPLALLLHAARPQAVVTQLSSMSGSPGQTVTITCTGSSSNIGGGYYLSWYQQLPGTAPKLLIYNVNNRASGVPNRFSGSKTGSLASLTITGLQAEDEADYYCGSYDESLSYVFGGGTHLTVLGGGGSAVQLVESGGGLVQPGGSLRLSCTASGFNFGLYAMSWVRQAPGKGLEWVSGINGGGGLTYYPDSMKGRFTISRDVAKNTLYLELNSLKIEDTAMYYCAKDPDMWTAFPNALDAWGQGTLVTVSSGSTSGSGKPGSGEGSTKGSYELTQPSAVSVSLGETARITCQGGNIGINYANWYQQKPGQAPVLVIYGDDRRPSGIPERFSGSISGGTATLTISGAQAEDEADYYCQSADSSPYVVFGGGTHLTVLGGGGSQVQLVESGGGLVQPGGSLRVSCAASGFTFKNYYMGWVRQAPGKGLEWISGINSGGEVTTYADSVKDRFTISRDNAKNTLYLQMNSLKPDDTALYYCAGGEGSSWYPDYYYGNGYWGEGTLITVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 141 - protein, 763 aa, synthetic construct
MALPVTALLLPLALLLHAARPNFMLTQPSAVSVSLGETARITCQGGNVGTSYASWYQQKPGQAPVLVIYGNSNRPSGIPERFSGSKSGGTATLTISGAQAEDEADYYCQSADSRGYVAFGGGTHLTVLGGGGSGGGGSGGGGSQVQLQESGGGLVQPGGSLRLSCAASGFTFSSYDITWVRQAPGKGLEWVSAINSGGNSTYYADSVKGRFTISRDNAKNTLYLQMNSLKTEDTAVYYCATAIEREEDPLWVGPEYNYWGQGTQVTVSSGGGGSGGGGSGGGGSGGGGSGGGGSSSELTQPSAVSVSLGQTARITCQGGNFGKYYAHWYQQKPGQAPVLVIYEDSERPSGIPERFSGSSSGDTATLTISGAQAEDEGDYYCHSAESSAAVFGGGTHLTVLGGGGSGGGGSGGGGSQVQLVQSGGGLVQPGGSLRLSCAASGFTFSSYAMSWVRQAPGKGLEWVSAINSGDSSTYYVDSVKGRFTISRDNAKNTLYLQLNSLKTEDTAMYYCAKDRIPGPYSDGGGFLGIVFGYWGQGTQVTISSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 142 - protein, 999 aa, synthetic construct
MALPVTALLLPLALLLHAARPSYELTQPSAVSVSLGETARITCQGGNIGINYANWYQQKPGQAPVLVIYGDDRRPSGIPERFSGSISGGTATLTISGAQAEDEADYYCQSADSSPYVVFGGGTHLTVLGGGGSGGGGSGGGGSAVQLVESGGGLVQPGGSLRLSCTASGFNFGLYAMSWVRQAPGKGLEWVSGINGGGGLTYYPDSMKGRFTISRDVAKNTLYLELNSLKIEDTAMYYCAKDPDMWTAFPNALDAWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPMADYKSYELTQPSAVSVSLGQTARITCQGGNLGTSYVHWYQQKPGQAPVLVIYGDDSRPSGIPERFSGSSSGGTATLTISGAQAEDEADYYCQSYDSSSSVFGGGTHLTVLGGGGSGGGGSGGGGSQVQLQESGGGLVQPGGSLRLSCAASGFTFSNNWMYWVRQAPGKGLEWVSSIATGDGSTNSADSVKGRFTISRDNTKNMLYLQMNSLKSEDTALYYCAKGVNIDHDVGAGDYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 143 - protein, 1005 aa, synthetic construct
MALPVTALLLPLALLLHAARPSYELTQPSAVSVSLGETARITCQGGNIGINYANWYQQKPGQAPVLVIYGDDRRPSGIPERFSGSISGGTATLTISGAQAEDEADYYCQSADSSPYVVFGGGTHLTVLGGGGSGGGGSGGGGSAVQLVESGGGLVQPGGSLRLSCTASGFNFGLYAMSWVRQAPGKGLEWVSGINGGGGLTYYPDSMKGRFTISRDVAKNTLYLELNSLKIEDTAMYYCAKDPDMWTAFPNALDAWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPMADYKSYELTQSPSVSVALRQAAKITCRGDNIETKYAYWYQQKPGQSPLLVIYKDDNRPSGIPERFSGSNSGNMATLTISGAQAEDEADYYCQVWDSSANWVFGGGTHLTVLGGGGSGGGGSGGGGSQVQLQESGGGLVQPGGSLRLSCAASGFTFSSYWMYWVRQAPGKGLEWVSAMNTGGGSTYYGDSVKGRFTVSRDNAKNTLALQMNSLKPEDTALYYCARSWGGMYSGGYYEYSGMDYWGKGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 144 - protein, 1004 aa, synthetic construct
MALPVTALLLPLALLLHAARPSSELTQPSAVSVSLGQTARITCQGGNFGKYYAHWYQQKPGQAPVLVIYEDSERPSGIPERFSGSSSGDTATLTISGAQAEDEGDYYCHSAESSAAVFGGGTHLTVLGGGGSGGGGSGGGGSQVQLVQSGGGLVQPGGSLRLSCAASGFTFSSYAMSWVRQAPGKGLEWVSAINSGDSSTYYVDSVKGRFTISRDNAKNTLYLQLNSLKTEDTAMYYCAKDRIPGPYSDGGGFLGIVFGYWGQGTQVTISSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPMADYKSYELTQPSAVSVSLGQTARITCQGGNLGTSYVHWYQQKPGQAPVLVIYGDDSRPSGIPERFSGSSSGGTATLTISGAQAEDEADYYCQSYDSSSSVFGGGTHLTVLGGGGSGGGGSGGGGSQVQLQESGGGLVQPGGSLRLSCAASGFTFSNNWMYWVRQAPGKGLEWVSSIATGDGSTNSADSVKGRFTISRDNTKNMLYLQMNSLKSEDTALYYCAKGVNIDHDVGAGDYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 145 - protein, 1010 aa, synthetic construct
MALPVTALLLPLALLLHAARPSSELTQPSAVSVSLGQTARITCQGGNFGKYYAHWYQQKPGQAPVLVIYEDSERPSGIPERFSGSSSGDTATLTISGAQAEDEGDYYCHSAESSAAVFGGGTHLTVLGGGGSGGGGSGGGGSQVQLVQSGGGLVQPGGSLRLSCAASGFTFSSYAMSWVRQAPGKGLEWVSAINSGDSSTYYVDSVKGRFTISRDNAKNTLYLQLNSLKTEDTAMYYCAKDRIPGPYSDGGGFLGIVFGYWGQGTQVTISSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPMADYKSYELTQSPSVSVALRQAAKITCRGDNIETKYAYWYQQKPGQSPLLVIYKDDNRPSGIPERFSGSNSGNMATLTISGAQAEDEADYYCQVWDSSANWVFGGGTHLTVLGGGGSGGGGSGGGGSQVQLQESGGGLVQPGGSLRLSCAASGFTFSSYWMYWVRQAPGKGLEWVSAMNTGGGSTYYGDSVKGRFTVSRDNAKNTLALQMNSLKPEDTALYYCARSWGGMYSGGYYEYSGMDYWGKGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 146 - protein, 21 aa, synthetic construct
MALPVTALLLPLALLLHAARP

SEQ ID NO: 147 - protein, 265 aa, Homo sapiens
MSEEVTYADLQFQNSSEMEKIPEIGKFGEKAPPAPSHVWRPAALFLTLLCLLLLIGLGVLASMFHVTLKIEMKKMNKLQNISEELQRNISLQLMSNMNISNKIRNLSTTLQTIATKLCRELYSKEQEHKCKPCPRRWIWHKDSCYFLSDDVQTWQESKMACAAQNASLLKINNKNALEFIKSQSRSYDYWLGLSPEEDSTRGMRVDNIINSSAWVIRNAPDLNNMYCGYINRLYVQYYHCTYKKRMICEKMANPVQLGSTYFREA

SEQ ID NO: 148 - protein, 6 aa, synthetic construct
SGGGGS

SEQ ID NO: 149 - protein, 25 aa, synthetic construct
GGGGSGGGGSGGGGSGGGGSGGGGS

SEQ ID NO: 150 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 151 - protein, 69 aa, synthetic construct
TTTPAPRPPTPAPTIASQPLSLRPEACRPAAGGAVHTRGLDFACDIYIWAPLAGTCGVLLLSLVITLYC

SEQ ID NO: 152 - protein, 42 aa, synthetic construct
KRGRKKLLYIFKQPFMRPVQTTQEEDGCSCRFPEEEEGGCEL

SEQ ID NO: 153 - protein, 45 aa, synthetic construct
TTTPAPRPPTPAPTIASQPLSLRPEACRPAAGGAVHTRGLDFACD

SEQ ID NO: 154 - protein, 24 aa, synthetic construct
IYIWAPLAGTCGVLLLSLVITLYC

SEQ ID NO: 155 - protein, 39 aa, synthetic construct
IEVMYPPPYLDNEKSNGTIIHVKGKHLCPSPLFPGPSKP

SEQ ID NO: 156 - protein, 27 aa, synthetic construct
FWVLVVVGGVLACYSLLVTVAFIIFWV

SEQ ID NO: 157 - protein, 41 aa, synthetic construct
RSKRSRLLHSDYMNMTPRRPGPTRKHYQPYAPPRDFAAYRS

SEQ ID NO: 158 - protein, 83 aa, synthetic construct
KRGRKKLLYIFKQPFMRPVQTTQEEDGCSCRFPEEEEGGCELRSKRSRLLHSDYMNMTPRRPGPTRKHYQPYAPPRDFAAYRS

SEQ ID NO: 159 - protein, 492 aa, synthetic construct
MALPVTALLLPLALLLHAARPNFMLTQPSAVSVSLGETARITCQGGNVGTSYASWYQQKPGQAPVLVIYGNSNRPSGIPERFSGSKSGGTATLTISGAQAEDEADYYCQSADSRGYVAFGGGTHLTVLGGGGSGGGGSGGGGSQVQLQESGGGLVQPGGSLRLSCAASGFTFSSYDITWVRQAPGKGLEWVSAINSGGNSTYYADSVKGRFTISRDNAKNTLYLQMNSLKTEDTAVYYCATAIEREEDPLWVGPEYNYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 160 - protein, 248 aa, synthetic construct
NFMLTQPSAVSVSLGETARITCQGGNVGTSYASWYQQKPGQAPVLVIYGNSNRPSGIPERFSGSKSGGTATLTISGAQAEDEADYYCQSADSRGYVAFGGGTHLTVLGGGGSGGGGSGGGGSQVQLQESGGGLVQPGGSLRLSCAASGFTFSSYDITWVRQAPGKGLEWVSAINSGGNSTYYADSVKGRFTISRDNAKNTLYLQMNSLKTEDTAVYYCATAIEREEDPLWVGPEYNYWGQGTQVTVSS

SEQ ID NO: 161 - protein, 107 aa, synthetic construct
NFMLTQPSAVSVSLGETARITCQGGNVGTSYASWYQQKPGQAPVLVIYGNSNRPSGIPERFSGSKSGGTATLTISGAQAEDEADYYCQSADSRGYVAFGGGTHLTVL

SEQ ID NO: 162 - protein, 126 aa, synthetic construct
QVQLQESGGGLVQPGGSLRLSCAASGFTFSSYDITWVRQAPGKGLEWVSAINSGGNSTYYADSVKGRFTISRDNAKNTLYLQMNSLKTEDTAVYYCATAIEREEDPLWVGPEYNYWGQGTQVTVSS

SEQ ID NO: 163 - protein, 493 aa, synthetic construct
MALPVTALLLPLALLLHAARPSYELTQSPSVSVALRQAAKITCRGDNIETKYAYWYQQKPGQSPLLVIYKDDNRPSGIPERFSGSNSGNMATLTISGAQAEDEADYYCQVWDSSANWVFGGGTHLTVLGGGGSGGGGSGGGGSQVQLQESGGGLVQPGGSLRLSCAASGFTFSSYWMYWVRQAPGKGLEWVSAMNTGGGSTYYGDSVKGRFTVSRDNAKNTLALQMNSLKPEDTALYYCARSWGGMYSGGYYEYSGMDYWGKGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 164 - protein, 249 aa, synthetic construct
SYELTQSPSVSVALRQAAKITCRGDNIETKYAYWYQQKPGQSPLLVIYKDDNRPSGIPERFSGSNSGNMATLTISGAQAEDEADYYCQVWDSSANWVFGGGTHLTVLGGGGSGGGGSGGGGSQVQLQESGGGLVQPGGSLRLSCAASGFTFSSYWMYWVRQAPGKGLEWVSAMNTGGGSTYYGDSVKGRFTVSRDNAKNTLALQMNSLKPEDTALYYCARSWGGMYSGGYYEYSGMDYWGKGTLVTVSS

SEQ ID NO: 165 - protein, 107 aa, synthetic construct
SYELTQSPSVSVALRQAAKITCRGDNIETKYAYWYQQKPGQSPLLVIYKDDNRPSGIPERFSGSNSGNMATLTISGAQAEDEADYYCQVWDSSANWVFGGGTHLTVL

SEQ ID NO: 166 - protein, 127 aa, synthetic construct
QVQLQESGGGLVQPGGSLRLSCAASGFTFSSYWMYWVRQAPGKGLEWVSAMNTGGGSTYYGDSVKGRFTVSRDNAKNTLALQMNSLKPEDTALYYCARSWGGMYSGGYYEYSGMDYWGKGTLVTVSS

SEQ ID NO: 167 - protein, 494 aa, synthetic construct
MALPVTALLLPLALLLHAARPQAVVTQLSSMSGSPGQTVTITCTGSSSNIGGGYYLSWYQQLPGTAPKLLIYNVNNRASGVPNRFSGSKTGSLASLTITGLQAEDEADYYCGSYDESLSYVFGGGTHLTVLGGGGSGGGGSGGGGSQVQLVESGGGLVQPGGSLRVSCAASGFTFKNYYMGWVRQAPGKGLEWISGINSGGEVTTYADSVKDRFTISRDNAKNTLYLQMNSLKPDDTALYYCAGGEGSSWYPDYYYGNGYWGEGTLITVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 168 - protein, 250 aa, synthetic construct
QAVVTQLSSMSGSPGQTVTITCTGSSSNIGGGYYLSWYQQLPGTAPKLLIYNVNNRASGVPNRFSGSKTGSLASLTITGLQAEDEADYYCGSYDESLSYVFGGGTHLTVLGGGGSGGGGSGGGGSQVQLVESGGGLVQPGGSLRVSCAASGFTFKNYYMGWVRQAPGKGLEWISGINSGGEVTTYADSVKDRFTISRDNAKNTLYLQMNSLKPDDTALYYCAGGEGSSWYPDYYYGNGYWGEGTLITVSS

SEQ ID NO: 169 - protein, 110 aa, synthetic construct
QAVVTQLSSMSGSPGQTVTITCTGSSSNIGGGYYLSWYQQLPGTAPKLLIYNVNNRASGVPNRFSGSKTGSLASLTITGLQAEDEADYYCGSYDESLSYVFGGGTHLTVL

SEQ ID NO: 170 - protein, 125 aa, synthetic construct
QVQLVESGGGLVQPGGSLRVSCAASGFTFKNYYMGWVRQAPGKGLEWISGINSGGEVTTYADSVKDRFTISRDNAKNTLYLQMNSLKPDDTALYYCAGGEGSSWYPDYYYGNGYWGEGTLITVSS

SEQ ID NO: 171 - protein, 487 aa, synthetic construct
MALPVTALLLPLALLLHAARPSYELTQPSAVSVSLGQTARITCQGGNLGTSYVHWYQQKPGQAPVLVIYGDDSRPSGIPERFSGSSSGGTATLTISGAQAEDEADYYCQSYDSSSSVFGGGTHLTVLGGGGSGGGGSGGGGSQVQLQESGGGLVQPGGSLRLSCAASGFTFSNNWMYWVRQAPGKGLEWVSSIATGDGSTNSADSVKGRFTISRDNTKNMLYLQMNSLKSEDTALYYCAKGVNIDHDVGAGDYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 172 - protein, 243 aa, synthetic construct
SYELTQPSAVSVSLGQTARITCQGGNLGTSYVHWYQQKPGQAPVLVIYGDDSRPSGIPERFSGSSSGGTATLTISGAQAEDEADYYCQSYDSSSSVFGGGTHLTVLGGGGSGGGGSGGGGSQVQLQESGGGLVQPGGSLRLSCAASGFTFSNNWMYWVRQAPGKGLEWVSSIATGDGSTNSADSVKGRFTISRDNTKNMLYLQMNSLKSEDTALYYCAKGVNIDHDVGAGDYWGQGTQVTVSS

SEQ ID NO: 173 - protein, 106 aa, synthetic construct
SYELTQPSAVSVSLGQTARITCQGGNLGTSYVHWYQQKPGQAPVLVIYGDDSRPSGIPERFSGSSSGGTATLTISGAQAEDEADYYCQSYDSSSSVFGGGTHLTVL

SEQ ID NO: 174 - protein, 122 aa, synthetic construct
QVQLQESGGGLVQPGGSLRLSCAASGFTFSNNWMYWVRQAPGKGLEWVSSIATGDGSTNSADSVKGRFTISRDNTKNMLYLQMNSLKSEDTALYYCAKGVNIDHDVGAGDYWGQGTQVTVSS

SEQ ID NO: 175 - protein, 5 aa, synthetic construct
SYDIT

SEQ ID NO: 176 - protein, 17 aa, synthetic construct
AINSGGNSTYYADSVKG

SEQ ID NO: 177 - protein, 17 aa, synthetic construct
AIEREEDPLWVGPEYNY

SEQ ID NO: 178 - protein, 11 aa, synthetic construct
QGGNVGTSYAS

SEQ ID NO: 179 - protein, 7 aa, synthetic construct
GNSNRPS

SEQ ID NO: 180 - protein, 10 aa, synthetic construct
QSADSRGYVA

SEQ ID NO: 181 - protein, 5 aa, synthetic construct
SYWMY

SEQ ID NO: 182 - protein, 17 aa, synthetic construct
AMNTGGGSTYYGDSVKG

SEQ ID NO: 183 - protein, 18 aa, synthetic construct
SWGGMYSGGYYEYSGMDY

SEQ ID NO: 184 - protein, 11 aa, synthetic construct
RGDNIETKYAY

SEQ ID NO: 185 - protein, 7 aa, synthetic construct
KDDNRPS

SEQ ID NO: 186 - protein, 10 aa, synthetic construct
QVWDSSANWV

SEQ ID NO: 187 - protein, 5 aa, synthetic construct
NYYMG

SEQ ID NO: 188 - protein, 17 aa, synthetic construct
GINSGGEVTTYADSVKD

SEQ ID NO: 189 - protein, 16 aa, synthetic construct
GEGSSWYPDYYYGNGY

SEQ ID NO: 190 - protein, 14 aa, synthetic construct
TGSSSNIGGGYYLS

SEQ ID NO: 191 - protein, 7 aa, synthetic construct
NVNNRAS

SEQ ID NO: 192 - protein, 10 aa, synthetic construct
GSYDESLSYV

SEQ ID NO: 193 - protein, 5 aa, synthetic construct
NNWMY

SEQ ID NO: 194 - protein, 17 aa, synthetic construct
SIATGDGSTNSADSVKG

SEQ ID NO: 195 - protein, 13 aa, synthetic construct
GVNIDHDVGAGDY

SEQ ID NO: 196 - protein, 11 aa, synthetic construct
QGGNLGTSYVH

SEQ ID NO: 197 - protein, 7 aa, synthetic construct
GDDSRPS

SEQ ID NO: 198 - protein, 9 aa, synthetic construct
QSYDSSSSV

SEQ ID NO: 199 - protein, 18 aa, synthetic construct
GSTSGSGKPGSGEGSTKG

SEQ ID NO: 200 - protein, 5 aa, synthetic construct
GGGGS

SEQ ID NO: 201 - protein, 15 aa, synthetic construct
GGGGSGGGGSGGGGS